(19) United States
(12) Patent Application Publication
(10) Pub. No.: US 2009/0048439 A1
(43) Pub. Date: Feb. 19, 2009

(54) Isolation of nucleic acids molecules using modified solid supports

(76) Inventors: William G. Weisburg, San Diego, CA (US); Elizabeth L. Mather, San Diego, CA (US); Marjan Haghnia, Carlsbad, CA (US)

Correspondence Address: Bell, Boyd & Lloyd LLP, 3580 Carmel Mountain Road, Suite 200, San Diego, CA, 92130, US

(21) Appl. No.: 12/221,750

(22) Filed: Aug. 5, 2008

Related U.S. Application Data
(60) Provisional application No. 60/963,748, filed on Aug. 6, 2007.

(51) Int. Cl. C07H 1/00; B01J 19/00

(52) U.S. Cl. 536/25.41; 422/68.1

(57) ABSTRACT

Provided are solid supports that contain at least one hydrophilic ligand; and at least one hydrophobic ligand, where amount of the at least one hydrophobic ligand on the solid support relative to the amount of the at least one hydrophilic ligand on the solid support is adjusted for binding target nucleic acid(s) from a sample onto the solid support and/or for eluting the bound target nucleic acid(s) from the solid support, so that the amount of target nucleic acid(s) bound to the solid support and/or recovered after elution from the solid support is about 5% to about 500% greater than the amount of target nucleic acid(s) bound to the solid support and/or recovered from the solid support in the absence of either the at least one hydrophobic ligand or the at least one hydrophilic ligand or both. The solid supports with ligands are used for isolation of nucleic acid molecules from samples.

### RELATED APPLICATIONS

Benefit of priority is claimed to U.S. Provisional Application Ser. No. 60/963,748, to William G. Weisberg, Elizabeth Mather, and Maijan Haghnia, entitled “Isolation of Nucleic Acids Molecules Using Modified Solid Supports,” filed Aug. 6, 2007.

This application is related to corresponding International Application No. [Attorney Docket No. 119359-00050/8004PC] to William G. Weisberg, Elizabeth Mather, and Marjan Haghnia, entitled “Isolation of Nucleic Acids Molecules Using Modified Solid Supports,” which also claims priority to U.S. Provisional Application Ser. No. 60/963,748.

Where permitted, the subject matter of each of the above-referenced applications is incorporated by reference in its entirety.

### FIELD OF INVENTION

Solid supports and methods for isolating nucleic acid molecules are provided.

### BACKGROUND

The isolation of nucleic acids from samples that are complex nucleic acid-containing materials such as bacteria, viruses, cells, tissues, blood, serum, oligonucleotide synthesis reaction mixtures or mixtures of more than one type and/or length of oligonucleotide, is an important step in molecular biology. Isolation can include purification of nucleic acids from other components such as reagents and by-products of synthetic reactions or, in the case of biological samples, proteins, monosaccharides, polysaccharides, lipids, RNA and cellular components, such as organelles and cell membranes. Isolation also can include separating one type of nucleic acid molecule (e.g., DNA, RNA or PNA; circular, linear or supercoiled) from others of a different type, or the separation of nucleic acids according to sequence or length. The isolated nucleic acid molecules can have several applications in molecular biology including organism or gene identification, recombinant expression systems and gene therapy. In addition, nucleic acids or oligonucleotides often must be purified for use as hybridization probes or in PCR reactions, where contaminating molecules of a different sequence can result in erroneous identification of a target nucleic acid, or amplification of the wrong target nucleic acid.

The isolation of nucleic acids on a solid support offers a rapid and simple means of purifying or separating nucleic acids from complex starting materials. In general, purification and/or separation of nucleic acids using solid supports entails two steps: capture onto the solid support and elution from the solid support. Previous uses of solid supports to purify or separate nucleic acids include supports with polar or hydrophilic surfaces, such as charged surfaces (e.g., carboxylate), or supports with hydrophobic groups, such as octadecylsilyl groups (U.S. Pat. No. 5,234,809; U.S. Pat. No. 5,705,628; McFarland et al., Nucl. Acids Res., 7(4):1067-1079 (1979)).

Solid supports having a charged or polar surface provide colloidal stability to the supports, especially small beads that tend to aggregate, and can modulate the binding and elution of nucleic acids from the supports. When solid supports with hydrophobic surfaces are used, it is believed that capture of nucleic acids is facilitated by hydrophobic interactions. Thus, each type of solid support, polar or hydrophobic, possesses some but not all of the features that are required for optimal binding and recovery of nucleic acids.

Maximizing the binding of nucleic acids from a sample onto the solid support is especially important when the sample contains very small amounts of the nucleic acid desired to be purified or separated. The binding, however, has to be facilitated without compromising the colloidal stability of the solid support and/or elution of the bound nucleic acids from the solid support. Hence, there is a need for solid supports and methods using solid supports that provide improved nucleic acid recovery by maximizing nucleic acid binding from a sample, while maintaining colloidal stability and the ability to elute the bound nucleic acids from the solid supports. It is among the objects herein to satisfy this and other needs.

### SUMMARY

Provided herein are solid supports and methods using solid supports that provide improve nucleic acid recovery by maximizing nucleic acid binding from a sample, while maintaining colloidal stability and the ability to elute the bound nucleic acids from the solid supports. The compositions, methods, combinations, kits and articles of manufacture provided herein contain a variety of component ingredients, steps of preparation, and biophysical, physical, biochemical or chemical parameters. As would be apparent to those of skill in the art, the compositions and methods provided herein include any and all permutations and combinations of the ingredients, steps and/or parameters described below and apparent to one of skill in the art.

Provided herein are solid supports for isolating nucleic acids with improved recovery compared to other supports. The solid supports provided herein are modified for improved nucleic acid recovery by adjusting their surface polarity and hydrophobicity to improve (1) capture of the nucleic acids onto the solid supports; and (2) elution of the nucleic acids from the solid supports, while maintaining colloidal stability of the solid supports compared to supports without such modifications.

In particular, provided are solid supports that contain at least one hydrophilic ligand; and at least one hydrophobic ligand. On the solid supports, the hydrophobic ligand(s) can be operatively linked, such as coupled or conjugated, to the hydrophilic ligand(s). The amount of the at least one hydrophobic ligand on the solid support relative to the amount of the at least one hydrophilic ligand on the solid support is adjusted for binding target nucleic acid(s) from a sample onto the solid support and/or for eluting the bound target nucleic acid(s) from the solid support, so that the amount of target nucleic acid(s) bound to the solid support and/or recovered after elution from the solid support is about 5% to about 500% greater than the amount of target nucleic acid(s) bound to the solid support and/or recovered from the solid support in the absence of either the at least one hydrophobic ligand or the at least one hydrophilic ligand or both.

Exemplary of such supports are those in which the amount of hydrophobic ligand(s) on the solid support relative to the amount of hydrophilic ligands on the solid support is from or from about 0.0001% to or to about 100%, from or from about 0.003% to or to about 70%, from or from about 0.005% to or to about 65%, from or from about 0.01% to or to about 50%, from or from about 0.03% to or to about 40%, from or from about 0.03% to or to about 33%, from or from about 0.1% to or to about 20%, from or from about 0.5% to or to about 10%, from or from about 0.01% to or to about 5%, from or from about 0.001% to or to about 3%, from or from about 0.0001% to or to about 3%, or from or from about 0.005% to or to about 2%. For example, the percentage of hydrophilic ligand(s) that is/are operatively linked to the hydrophobic ligand(s) can be from or from about 0.0001% to or to about 100%.

Provided are solid supports that contain only one hydrophilic ligand and only one hydrophobic ligand or only one hydrophilic ligand and hydrophobic ligands or hydrophilic ligands and only one hydrophobic ligand. Hydrophilic ligands include carboxylate and hydrophobic ligands include aliphatic amines. For example, the hydrophilic ligand is a carboxylate and the hydrophobic ligand is an amine, and the amine is operatively linked to the carboxylate and the operative linkage is via an amide bond. Aliphatic amines can be selected from among, for example, polypropylamine, propylamine hydrochloride, octylamine, butoxypropylamine, butylamine, 2-(2-aminoethoxy)ethanol, NH₂(CH₂)ₖ—O-{(CH₂ CH₂O)ₗ}ₘ-MGB, NH₂(CH₂)₆—O-Tₙ-MGB and NH₂(CH₂)ₖ—O—(CH₂ CH₂O)ₗ-Tₙ, where: k is an integer between 1 and 10; l is an integer between 1 and 10; m is an integer between 1 and 3; n is an integer between 1 and 10; T is thymidine; and MGB is a DNA Minor Groove Binder.

The solid supports can be in any form, such as in the form of beads. The solid supports can be magnetic or paramagnetic, such magnetic or paramagnetic beads. The solid supports can be fabricated from any suitable material that is compatible with nucleic acids and, where necessary, the processes herein. Exemplary of such material is agarose, cellulose, nitrocellulose, cellulose acetate, dextran, polysaccharides, glass, silica, gelatin, polyvinyl pyrrolidone, rayon, nylon, polyethylene, polypropylene, polybutylene, polycarbonate, polyesters, polyamides, vinyl polymers, polyvinyl alcohols, polystyrene, carboxylate-modified polystyrene, polystyrene cross-linked with divinylbenzene, acrylic resins, acrylates, acrylic acids, acrylamides, polyacrylamides, polyacrylamide blends, co-polymers of vinyl and acrylamide, methacrylates, methacrylate derivatives and co-polymers thereof, and mixtures of any of these materials.

In some examples, the hydrophilic ligand is a carboxylate and the hydrophobic ligand is propylamine. In such instances, the percentage of carboxylate residues operatively linked to propylamine can be from about or at 0.1% to about or at 20%, such as from about or at 15% to about or at 20%; from about or at 15% to about or at 17%; from about or at 1% to about or at 10%; or from about or at 1% to about or at 2%. In some examples, it is about or at 1.7%, 6.7%, 16.7% or 100%. The hydrophobic ligand also can be propylamine hydrochloride. The percentage of carboxylate residues operatively linked to propylamine hydrochloride can be from about or at 1% to about or at 10%, such as from about or at 1% to about or at 2%, such as about 1.7%. In other examples, the hydrophobic ligand is an octylamine. The percentage of carboxylate residues operatively linked to octylamine can be from about or at 0.1% to about or at 20%, such as from about or at 1% to about or at 10%, such as about 6.7%. In further examples, the hydrophobic ligand is the amine NH₂(CH₂)₆—O—P(═O)(O⁻)—O—(CH₂ CH₂ O)₆—P(═O)(O—)—O-MGB or the amine NH₂(CH₂)₆—O—P(═O)(O⁻)—O-TTTTTT-O—P(═O)(O⁻)—O-MGB, where MGB is:

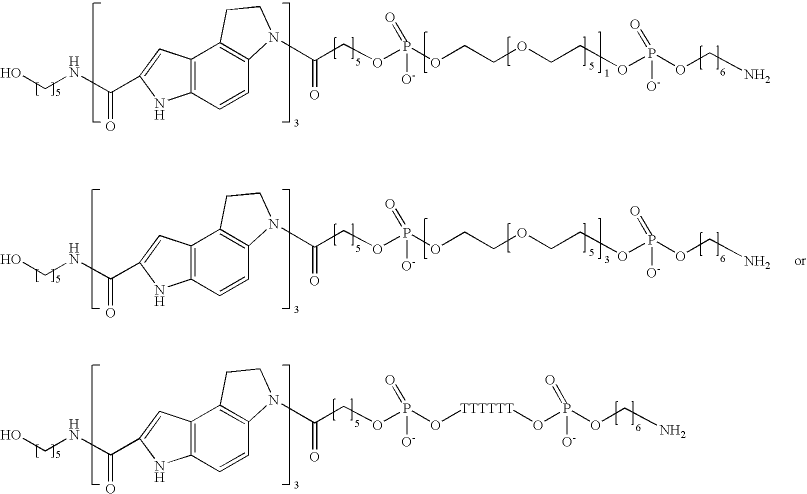

In such examples, the percentage of carboxylate residues operatively linked to the amine is from about or at 5% to about or at 20%. In other examples, the percentage of carboxylate residues operatively linked to the amine is from about or at 0.5% to about or at 5%, such as 0.5%, 1% or 2%.

Exemplary of any of the solid supports above and below, are those in which the aliphatic amine is NH₂(CH₂)ₖ—O-{(CH₂ CH₂O)ₗ}ₘ-MGB or NH₂(CH₂)₆—O-Tₙ-MGB, and the minor groove binder (MGB) is selected from among netropsin, distamycin, lexitropsin, mithramycin, chromomycin A₃, olivomycin, anthramycin, sibiromycin, pentamidine, stilbamidine, berenil, CC-1065, Hoechst 33258, 4′-6-diamidino-2-phenylindole (DAPI),

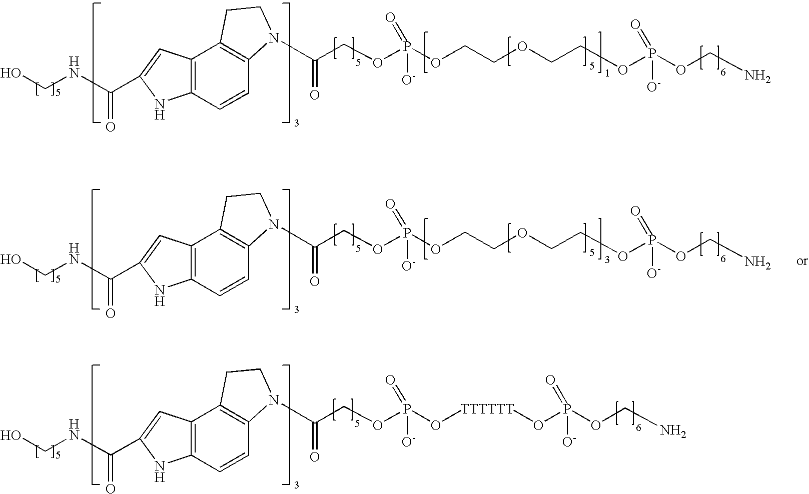

Also provided are methods for isolating nucleic acids from any sample, including biological tissues and fluids and purified forms thereof or compositions derived therefrom.

Provided are combinations of a solid support, such as of the solid supports provided herein, a chaotropic substance; and an elution buffer. Also provided are combinations containing: a solid support; hydrophilic ligand; and a hydrophobic ligand; or combinations containing a solid support comprising a hydrophilic ligand; and a hydrophobic ligand. The combinations also can include a coupling reagent. Exemplary of the combinations are those in which the hydrophilic ligand carboxylate, the hydrophobic ligand is an amine, and the coupling reagent is carbodiimide.

Also provided are kits containing the combinations and a packaging material for the combination and, optionally, a label that indicates that the combination is for use in nucleic acid isolation, optionally instructions for use, and optionally additional reagents and/or materials for performing the methods herein.

Provided are methods for isolating nucleic acid molecules by contacting any of the supports with linked or coupled ligands provided herein with a sample containing a nucleic acid molecule, such as DNA, RNA and mixtures thereof whereby the nucleic acid molecule is captured by the support. Any method for eluting or removing captured nucleic acid molecules can be used. For example, provided is a method for isolating nucleic acid molecules, by: contacting a solid support of with a sample that contains or is suspected of containing nucleic acid molecules, including target nucleic acid molecules; mixing the components of the previous step in a solution comprising a chaotropic buffer and alcohol, where the amounts of the chaotropic substance and alcohol are adjusted for binding or capturing the nucleic acid molecules onto the solid support; separating the solid support containing the bound target nucleic acid molecules from the solution; washing the solid support containing bound or captured nucleic acid molecules; and combining the resulting washed solid support with a second solution for eluting the bound target nucleic acids, whereby the target nucleic acid molecules are purified from the sample. The methods can be for purifying target nucleic acid molecules from a sample. Any of the solid supports provided herein can be used in such methods.

In another method of isolating nucleic acid molecules, such as target nucleic acid molecules, a solid support is provided or identified for use and the method includes steps of: adjusting the hydrophilicity of the solid support by adjusting the amount of hydrophilic ligand on the solid support; adjusting the hydrophobicity of the solid support by adjusting the amount of hydrophobic ligand on the solid support; and binding the target nucleic acid molecules from the sample onto the resulting solid support; wherein the hydrophobicity of the resulting solid support is adjusted for binding the nucleic acid molecules onto the solid support and the hydrophilicity of the solid support is adjusted for maintaining colloidal stability of the solid support and for eluting the nucleic acid molecules off the solid support, whereby the amount of target nucleic acid(s) bound to the solid support and/or recovered after elution from the solid support is about 5% to about 500% greater than the amount of target nucleic acid(s) bound to the solid support and/or recovered from the solid support in the absence of either the at least one hydrophobic ligand or the at least one hydrophilic ligand.

For the methods, the hydrophilic and/or hydrophobic ligand(s) operatively linked, such as coupled, such as covalently, to the solid support. In any of the methods, the percentage of hydrophilic ligand(s) that is/are operatively linked to the hydrophobic ligand(s) can be from about 0.0001% to about 100%.

The methods can be for separating target nucleic acid molecules from each other according to type, length or sequence. The amount or concentration of the chaotropic substance and/or the alcohol and/or the elution buffer is adjusted so that the target nucleic acid molecules are eluted sequentially according to type, length or sequence.

Also provided are methods for preparing the solid supports provided herein, by: identifying a solid support coated with a hydrophilic ligand; and operatively linking a hydrophobic ligand to the identified solid support; where the hydrophobicity of the solid support is adjusted for binding the target nucleic acids from the sample onto the solid support, whereby the amount of target nucleic acid(s) bound to the solid support and/or recovered after elution from the solid support is about 5% to about 500% greater than the amount of target nucleic acid(s) bound to the solid support and/or recovered from the solid support in the absence of either the at least one hydrophobic ligand or the at least one hydrophilic ligand.

### DETAILED DESCRIPTION

### A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, “about” with reference to the amount also means the amount specified. Hence the statement about 5 mg means about 5 mg or 5 mg. “About” means within typical experimental error for the application or purpose intended.

As used herein, the term “ligand” refers to any molecule having a nucleic acid or protein binding functionality. The nucleic acid or protein-binding functionality of the ligands provided herein can be conjugated or coupled to a solid support, or can be in operative linkage with a solid support, or can form part of the material constituting the solid support. The nucleic acid or protein binding functionality of the ligand generally is located on the surface of the solid support or at a location on the solid support whereby the nucleic acid or protein can form a conjugate or operative linkage or can couple with the binding functionality. Ligands include hydrophilic and hydrophobic ligands.

As used herein, a “hydrophilic” or “polar” ligand is a ligand that has a charge or is charge-polarized. A hydrophilic ligand as used herein has either a charged functional group, such as a carboxylate or ammonium, or a charge-polarized bond, such as hydroxyl or sulfhydryl that provides a charge to the ligand. Hydrophilic ligands can bond with water and other polar solvents including alcohols, amines, amides, acids, carboxylic acids, esters, nitriles, ketones, glycols and glycol ethers, through hydrogen bonds or ionic interactions. A hydrophilic ligand also has greater solubility in polar solvents than in non-polar solvents.

As used herein, a “hydrophobic” or “non-polar” ligand refers to a ligand that is not charged or charge-polarized, or is not sufficiently charged or charge-polarized to bond with water or other polar solvents. Hydrophobic ligands can associate with each other or with other non-polar molecules or solvents in the presence of water or a polar solvent, through hydrophobic interactions. A hydrophobic ligand generally also is more soluble in non-polar solvents than in polar solvents. Examples of non-polar solvents include alkanes such as hexane, alkyl ethers such as diethyl ether, aromatic hydrocarbons such as benzene and alkyl halides such as methylene chloride and carbon tetrachloride, mono-, di- and triglycerides, fatty acids, such as oleic, linoleic, palmitic, stearic, conjugated forms thereof and their esters.

The term “hydrophilicity” as used herein refers to the solubility of a ligand in a polar solvent relative to its solubility in a non-polar solvent. For example, a hydrophilic ligand as used herein would have a solubility of about or greater than 1.5, 2.0, 2.5, 3.0, 5.0, 10, 20, 40, 80, 100, 200, 500, 1000, 5000, 10,000, 10⁶ or greater-fold solubility in a polar solvent relative to a non-polar solvent.

The term “hydrophobicity” as used herein refers to the solubility of a ligand in a non-polar solvent relative to its solubility in a polar solvent. For example, a hydrophobic ligand as used herein would have a solubility of about or greater than 1.5, 2.0, 2.5, 3.0, 5.0, 10, 20, 40, 80, 100, 200, 500, 1000, 5000, 10,000, 10⁶ or greater-fold solubility in a non-polar solvent relative to a polar solvent.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “oligonucleotide” or “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). “Oligonucleotide” also is used herein synonymously with “polynucleotide,” although those in the art will recognize that oligonucleotides, for example, PCR primers, generally are less than about fifty to one hundred or 120 nucleotides in length.

Nucleotide analogs contained in a polynucleotide can include, for example, mass modified nucleotides, which allows for mass differentiation of polynucleotides; nucleotides containing a detectable label such as a fluorescent, radioactive, luminescent or chemiluminescent label, which allows for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis.

A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond, or the like, and is capable of being extended by a polymerase. Peptide nucleic acid molecules can be prepared using well known methods (see, for example, Weiler et al., Nucleic Acids Res. 25:2792-2799 (1997)).

A polynucleotide can be a portion of a larger nucleic acid molecule, for example, a portion of a gene, which can contain a polymorphic region, or a portion of an extragenic region of a chromosome, for example, a portion of a region of nucleotide repeats such as a short tandem repeat (STR) locus, a variable number of tandem repeats (VNTR) locus, a microsatellite locus or a minisatellite locus. A polynucleotide also can be single stranded or double stranded, including, for example, a DNA-RNA hybrid, or can be triple stranded or four stranded. Where the polynucleotide is double stranded DNA, it can be in an A, B, L or Z configuration, and a single polynucleotide can contain combinations of such configurations.

As used herein, “target nucleic acid” refers to any nucleic acid of interest or to a portion thereof. For example, a target nucleic acid can be a polymorphic region of a gene or a region of a gene potentially having a mutation. Target nucleotide sequences include, but are not limited to, nucleotide sequence motifs or patterns specific to a particular disease and causative thereof; nucleotide sequences specific as a marker of a disease; nucleotide sequences specific to a pathogenic organism or other microorganism; and nucleotide sequences of interest for research purposes, but that may not have a direct connection to a disease. A target nucleotide sequence can be any region of contiguous nucleotides that encodes a polypeptide of at least 2, or 3, or 4, or at least 5 amino acids. A target nucleic acid encodes a target polypeptide.

As used herein “isolated” or “purified” refers to samples where the analyte of interest is separated from other molecules or substances that are present in the source from which the analyte is obtained. As used herein, “isolate” and “purify”, and immediate variations thereof, such as “isolation” and “purification”, are used interchangeably and, when applied to a nucleic acid, refer to the process of removing the nucleic acid from its immediate environment or milieu so that it is substantially free (about or greater than 70%, 80%, 90%, 95%, 96%, 97%, 98%, 99% or 100%) of extraneous or unwanted chemicals, genetic material, proteins, degradation products, or other unwanted materials. The nucleic acid can be isolated or purified from buffers, biological samples, such as for example, plasma, blood, or nasopharyngeal specimens, and other liquid and solid samples, and can be isolated or purified from eukaryotic, prokaryotic or viral material.

As used herein, “isolated” also includes a nucleic acid or polypeptide or other analyte that is substantially free (about or greater than 70%, 80%, 90%, 95%, 96%, 97%, 98%, 99% or 100%) of cellular material or viral material including proteins, monosaccharides, polysaccharides, lipids, RNA and cellular components, such as organelles and cell membranes, or culture medium when produced by recombinant techniques, or chemical precursors or other chemicals when chemically synthesized. Moreover, an “isolated nucleic acid” is includes nucleic acid fragments that are not naturally occurring as fragments and would not be so-found in the natural state.

“Isolation” as used herein also includes separating one type of nucleic acid molecule (e.g., DNA, RNA or PNA; circular, linear or supercoiled) from others of a different type, or the separation of nucleic acids according to sequence or length. The term “isolated” is also used herein to refer to polypeptides which are isolated from other cellular proteins and is meant to encompass both purified and recombinant polypeptides.

As used herein, the term “sample” refers to any liquid or solid material to be examined in the processes described herein. For example, a sample can be a solution containing eukaryotic or prokaryotic cells or cellular material, or virus or viral material, or bacteria or bacterial material, or microorganisms or pathogens. A sample can be essentially water, or a buffered solution or be composed of any artificially introduced chemicals, and may or may not contain nucleic acids. As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed For example, isolated nucleic acids that are amplified constitute a biological sample. Biological samples can include biological solid material or biological fluid or a biological tissue. Examples of biological solid materials include tumors, cell pellets, biopsies. Examples of biological fluids include urine, blood, plasma, serum, sweat, saliva, semen, stool, sputum, cerebral spinal fluid, mouth wash, tears, mucus, sperm, amniotic fluid or the like. Biological tissues are aggregates of cells, usually of a particular kind, together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Also included are soil, water and other environmental samples including industrial waste and natural bodies of water (lakes, streams, rivers, oceans) that can contain viruses, bacteria, fungi, algae, protozoa and components thereof.

As used herein, the term “colloid” refers to a dispersion of solid particles, such as beads, in a liquid, such as a solution containing a nucleic acid to be captured on the beads. The term “colloidal stability” refers to a colloid in which the solid particles are not substantially aggregated. For example, a stable colloid is one in which about 30%, 25%, 20%, 15%, 10%, 5%, 4%, 3%, 2%, 1% 0.5% or less of the solid particles, such as beads, have formed aggregates.

As used herein, “aggregates” refers to the association of one or more particles, such as beads, which inhibits their ability to disperse and form a colloid in solution.

As used herein, the term “paramagnetic” refers to the exhibition of the property of being attracted by a magnet, and of assuming a position parallel to that of an externally applied magnetic force, but not of becoming permanently magnetized. As used herein, the term “superparamagnetic” refers to the exhibition of the property of being attracted by a magnet, and of assuming a position parallel to that of an externally applied magnetic force, but not of becoming permanently magnetized, even at temperatures below the Curie temperature or the Neel temperature. The term “paramagnetic” also is used herein essentially synonymously with, and as an abbreviation of, “superparamagnetic”, although those in the art recognize the distinctions set forth above.

As used herein, the term “bead” refers to a small mass that can be composed of alumina, glass, silica, latex, plastic or any polymeric material, and be of any size and shape, but are generally polymeric and spherical and from about 0.05 to about 500 microns, from about 0.25 to about 200 microns, from about 0.5 to about 100 microns, from about 0.5 to about 25 microns, from about 0.5 to about 1.5 microns, or about 0.5 to about 1.0 micron in size. Additionally, and for purposes herein, the bead can be made magnetically responsive by heterocoagulation of paramagnetic or, typically, superparamagnetic substances, such as for example, magnetite, to the surface of the bead.

As used herein, a “solid support” is an insoluble material to which reagents or material can be attached so that they can be readily separated from the original solution. A solid support can be a bead. In other embodiments, the solid support can be an insoluble material to which the beads are attached or associated, such as for example, by magnetic forces. For example, paramagnetic beads can be contained in a solid support such as, but not limited to, microfuge tubes, columns, or multi-well microtiter plates, to which a magnetic force is applied, such as by samarium, cobalt or neodymium magnet, thus attaching the beads to the solid support until removal of the magnetic force releases the beads.

A support or solid support refers to the material to which an analyte can be linked. The term “solid support” means a non-gaseous, non-liquid material having a surface. Thus, a solid support can be a flat surface constructed, for example, of glass, silicon, metal, plastic or a composite; or can be in the form of a bead such as a silica gel, a controlled pore glass, a magnetic or cellulose bead; or can be in the form of a column, such as those used in chromatography; or can be a pin, including an array of pins suitable for combinatorial synthesis or analysis.

A variety of materials can be used as the solid support. The support materials include any material that can act as a support for attachment of the molecules of interest. Such materials are known to those of skill in this art. These materials include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and co-polymers, other polymers and co-polymers with various functional groups, latex, butyl rubber and other synthetic rubbers, silicon, glass, paper, natural sponges, insoluble protein, surfactants, red blood cells, metals, metalloids, magnetic materials, or other commercially available media.

As used herein, “operatively linked” or “linked” means that the linkage between two separate entities produces an appropriate and expected effect. For example, when used in the phrase “the carboxylated beads are operatively linked to a propylamine molecule”, it means that the propylamine ligand and the beads are bonded by covalent forces as predicted by the known chemical properties of the beads and the ligand i.e. the hydroxyl group on the beads was replaced with the amino group on the ligand to form a covalent amide bond between the beads and the ligand. “Operatively linked” can refer to one or more covalent bonds including, but not limited to, an amide bond, disulphide bond and thioether bond, or refer to non-covalent interactions including, but not limited to, ionic interactions or hydrophobic interactions

As used herein, “coupled” refers to the joining, pairing, or association of two or molecules or entities. The association can be through covalent bonds including, but not limited to, an amide bond, disulphide bond or thioether bond, or through non-covalent interactions including, but not limited to, ionic interactions or hydrophobic interactions. For example, a bead can be coupled to a ligand. In some instances, the two entities that are coupled are operatively linked. In such instances, “coupled” can be used synonymously with “operatively linked”.

As used herein, the term “conjugated” refers to stable attachment, typically ionic or covalent attachment. Among conjugation couplers are: streptavidin- or avidin- to biotin interaction; hydrophobic interaction; magnetic interaction (e.g., using functionalized magnetic beads, such as DYNABEADS®, which are streptavidin-coated magnetic beads sold by Dynal, Inc., Great Neck, N.Y. and Oslo Norway); polar interactions, such as wetting associations between two polar surfaces or between oligo/polyethylene glycol; formation of a covalent bond, such as an amide bond, disulfide bond, thioether bond, or via crosslinking agents; and via an acid-labile or photocleavable linker. The terms “operatively linked,” “linked,” conjugated” and “coupled” can be used interchangeably herein, depending on the nature of the modified solid support.

As used herein, a “combination” refers to any association between two or more items or components for a common purpose. Thus, a combination for modifying solid supports to increase their hydrophobic surface can contain a magnetic bead coated with a charged/hydrophilic ligand, such as carboxylate, and a hydrophobic ligand, such as an alkylamine or a minor groove binding ligand (MGB ligand) amine, and can further containing a carbodiimide coupling reagent for modifying the surface of the solid support with the hydrophobic ligand. A combination for isolating nucleic acids using a solid support can contain a modified solid support as provided herein and further contain one or more reagents including a chaotropic substance, a binding buffer, an elution buffer, or reagents to make the modified solid support and/or the reagents.

As used herein, a “chaotropic substance” refers to any substance capable of altering the secondary, tertiary and/or quaternary structure of nucleic acids or proteins, while leaving at least the primary structure intact. Examples of chaotropic substances include, but are not limited to, guanidinium isothiocyanate, guanidine hydrochloride, sodium iodide, potassium iodide, sodium isothiocyanate, urea, or combinations thereof.

As used herein, a “kit” refers to a combination in which items or components are packaged optionally with instructions for use and/or reagents and apparatus for use with the combination.

As used herein, “modulate” and “modulation” refer to a change in the amount of nucleic acid or protein bound to/captured by a solid support, or a change in the amount of nucleic acid or protein eluted from the solid support, or a change in the nucleic acid or protein recovery from binding the nucleic acid or protein to the solid support, followed by its elution from the solid support. Modulation can be context dependent and typically modulation is compared to a designated state, for example, the amount of nucleic acid recovered from a bead coated with a charge, such as carboxylated bead, relative to the amount recovered from a carboxylated bead in which a fraction of the carboxylate groups are modified with a hydrophobic ligand.

### B. Modified Solid Supports

Provided herein are modified solid supports for isolating nucleic acids or proteins from a sample. The solid supports are modified to provide a hydrophilic ligand and a hydrophobic ligand. The hydrophilic and hydrophobic ligands on the solid support can form part of the polymer or other substance that constitutes the material forming the solid support, or can be covalently or non-covalently linked to the solid support.

The hydrophilic ligand can provide colloidal stability to dispersions of the solid support or particles thereof in the sample, which can be a solvent or solution in which the nucleic acid or protein is present. The colloidal stability can prevent the formation of aggregates of the solid support, which would decrease the available surface area for capturing nucleic acids or proteins from the sample. The hydrophilic ligand also can modulate binding of nucleic acids or proteins to the solid support and elution of nucleic acids or proteins from the solid support. The hydrophobic ligand provides a hydrophobic surface on the solid support for binding nucleic acids or other biomolecules from the sample.

The relative amounts of the hydrophilic and hydrophobic ligands on the solid support are adjusted to maximize recovery of nucleic acids, proteins or other biomolecules from the sample. For example, the amount of the hydrophobic ligand on the solid support should be sufficient to capture small amounts of nucleic acids from the sample onto the solid support, yet not be sufficient to inhibit elution of bound nucleic acids from the solid support and reduce net recovery of the isolated nucleic acids. Similarly, for example, the amount of hydrophilic ligand on the solid support should be sufficient to maintain colloidal stability of the solid support in the sample containing the nucleic acid, and to facilitate elution of bound nucleic acid from the solid support, yet not be sufficient to inhibit binding of nucleic acids to the solid support.

The hydrophilic and hydrophobic ligands on the solid support can each independently be conjugated or coupled to the solid support, or can be part of the material forming the solid support. Alternately, the relative hydrophilicity or hydrophobicity of the solid support can be adjusted by modifying a percentage of the hydrophilic ligands on the solid support with a hydrophobic ligand, or by modifying a percentage of the hydrophobic ligands on the solid support with a hydrophilic ligand. The modification can be through operative linkage or conjugation or coupling of the hydrophobic and hydrophilic ligands with each other. Such modification converts a percentage of hydrophilic ligands present on a solid support to generate a partial hydrophobic surface, or converts a percentage of hydrophobic ligands present on a solid support to generate a partial hydrophilic surface.

The relative amounts of hydrophilic and hydrophobic ligands on the solid support are adjusted depending on the type of ligand, to provide maximum nucleic acid, protein or other biomolecule recovery. Exemplary amounts of hydrophobic ligand on the solid support relative to hydrophilic ligand on the solid support are from about 0.0001% to about 100%, typically from about 0.003% to about 70%, from about 0.005% to about 65%, from about 0.01% to about 50%, from about 0.03% to about 40%, from about 0.03% to about 33%, from about 0.1% to about 20%, from about 0.5% to about 10%, from about 0.01% to about 5%, from about 0.001% to about 3%, from about 0.0001% to about 3%, or from about 0.005% to about 2%.

The relative amounts of the hydrophobic and hydrophilic ligands on the solid support are adjusted so that nucleic acid or protein or other biomolecule capture on the solid support, elution from the solid support, or net recovery is increased by about 5%, 7%, 10%, 15%, 20%, 25%, 30%, 33%, 40%, 45%, 50%, 55%, 60%, 65%, 70%, 75%, 80%, 85%, 90%, 95%, 100%, 110%, 115%, 120%, 125%, 130%, 135%, 140%, 145%, 150%, 155%, 160%, 165%, 170%, 175%, 180%, 185%, 190%, 195%, 200% or greater, relative to recovery in the presence of the hydrophilic ligand or hydrophobic ligand alone.

The modified solid supports provided herein can be used to isolate very small or very large amounts of nucleic acids or other biomolecules from a sample, from picomolar to nanomolar to micromolar to millimolar to molar concentrations. Exemplary concentrations can range from about 10⁻¹² M to about 1M or greater, from about 10⁻¹⁰ M to about 1M or greater, from about 10⁻⁹ M to about 0.5M, from about 10⁻⁶ M to about 0.5M, from about 10⁻⁴ M to about 0.3M, from about 10⁻⁹ M to about 10⁻⁴ M, from about 10⁻⁹ M to about 10⁻⁶ M, or from about 10⁻¹² M to about 10⁻⁶ M. The sample volume can range from about 0.001 ml to about 50, 100, 200 or 1000 ml or greater, from about 0.002 ml to about 20 ml, from about 0.003 ml to about 10 ml, from about 0.003 ml to about 7.5 ml, from about 0.003 ml to about 5 ml, from about 0.05 ml to about 3 ml, or from about 0.01 ml to about 1 ml.

1. Solid Supports

The solid support used to isolate nucleic acid molecules, proteins or other biomolecules can be in various forms, including, but not limited to, particles, microparticles, fibers, beads, membranes, sheets, gels, filters, capillaries, test tubes, and microtiter strips, tubes, plates or wells, that have sufficient surface area to permit binding of the nucleic acid molecules. Exemplary materials present a high surface area for binding the nucleic acid, and be regular or irregular in shape, and/or porous or non-porous. Solid supports in the form of particles, microparticles or beads additionally can contain a magnetically responsive portion, such as a magnetically responsive core, or a magnetically responsive shell.

The substrate of the solid support can be composed of a matrix of a material, including, but not limited to, silica, silica carbide, silica nitrate, glass, titanium, dioxide, aluminum oxide, zirconium oxide, carbon, charcoal, graphite, insoluble synthetic polymers and insoluble polysaccharides. Synthetic polymers are homopolymers or copolymers of one or more ethylenically unsaturated monomer units and can be crosslinked or non-crosslinked. Monomer units can include, but are not limited to, acrylamides, styrenes, alkyl-substituted styrenes, acrylates, methacrylates, acrylic acid, methacrylic acid, vinyl chloride, vinyl acetate, butadiene and isoprene.

Where the solid support is in the form of a bead, the bead can be a variety of shapes, which can be regular or irregular. In some instances, the bead is polymeric and spherical. The size is generally is such that their separation from solution, for example by filtration, centrifugation or magnetic separation, is readily accomplished. Additionally, the beads should not be so large as to minimize the surface area available for nucleic acid binding, or interfere with its functionality for microscale uses. A suitable size range can be from or about 0.05 to about 500 or 500 microns, from or about 0.25 or 0.25 to about 200 or about 200 microns, from or about 0.5 to about or 100 microns, from or about 0.5 to about 25 microns, from or about 0.5 to about 1.5 microns, or about 0.5 to about 1.0 micron in size. Beads can be manufactured or prepared from a variety of materials that are compatible with the procedures and nucleic acid molecules. Examples of bead materials include, but are not limited to, silica, glass, dextran, polystyrene, polypropylene, nylon, polyethylene, polycarbonate, polyamide, polyvinylidenediflouride (PVDF) agarose and acrylamide. Also, the beads can have a metal surface, such as for example, steel, gold, silver, aluminum, silicon and copper.

Additionally, the beads can be made magnetically responsive. This can be achieved by, for example, heterocoagulation of paramagnetic substances, including, but not limited to, superparamagnetic substances, such as magnetite, to the surface of the bead (see, e.g. U.S. Pat. No. 5,648,124). Paramagnetic beads are attracted by a magnet and assume a position parallel to that of the externally applied magnetic force, but do not become permanently magnetized. This facilitates the concentration and purification of the paramagnetic beads from the solution in which they reside, and by extension, the concentration and purification of compounds, such as for example, nucleic acid molecules, that are bound to the paramagnetic beads. For example, the paramagnetic beads can be contained in a test tube to which an external magnetic field is applied by way of an embedded rare earth (e.g. neodymium) magnet. The paramagnetic beads are attracted to the magnet and concentrated, facilitating removal of any solution from the tube and the beads. Removal of the magnet releases the paramagnetic beads, which can then be easily resuspended without any magnetically-induced aggregation occurring. Paramagnetic particles suitable for the isolation methods described herein contain a magnetite rich core and also are encapsulated by a pure polymer shell. In some instances, the paramagnetic microparticles have about 40% magnetite content by weight. Paramagnetic particles comprising too little magnetite are only weakly attracted to the magnets used by those of skill in the art to accomplish magnetic separations.

The magnetic metal oxide core can be iron oxide, with iron as a mixture of Fe²⁺ and Fe³⁺ at a ratio that can vary from about 0.5/1 to about 4/1. The use of encapsulated paramagnetic microparticles, having no exposed iron, or Fe₃O₄ on their surfaces, eliminates the possibility of iron interfering with various enzymatic functions in certain downstream manipulations of the isolated nucleic acid. Polymer encapsulation can be effected by directly applying the polymer coating, or by adding the desired monomer with a polymerization initiator, to the paramagnetic bead.

Monomers useful for preparing the outer polymeric shell include, but are not limited to, acidic monomers such as acrylic acid, methacrylic acid, fumaric acid, maleic acid, methacrylic acid, itaconic acid, vinyl acetic acid, 4-pentenoic acid, undecylenic acid, and salts thereof; basic monomers such as aminoethylmethacylate, dimethylaminoethyl methacrylate, t-butylaminoethyl methacrylate, pyrrole, N-vinyl carbazole, vinylpyridine, and salts thereof; hydrophobic neutral monomers such as methyl acrylate, ethyl acrylate, methyl methacrylate, ethyl methacrylate, styrene, methylstyrene, ethylstyrene, vinylnaphthalene, and homologs thereof. Paramagnetic beads also can be encapsulated in a silane coat. Polymer encapsulated paramagnetic beads are available from a variety of commercial sources, including, but not limited to, dextran-coated beads such as MagMAX™ magnetic beads (Ambion, Inc., Texas), carboxylated magnetic beads (e.g. Sera-Mag™ Microparticles, Seradyn, Ind. and BioMag® COOH beads, PerSeptive Diagnostics), magnetic microparticles coated with thiol groups (PerSeptive Diagnostics), magnetic microparticles coated with streptavidin (BioMag® Streptavidin from PerSeptive Diagnostics), and Dynabeads® M-270 (Dynal Inc).

Exemplary solid supports are provided in the following patents, published patent applications and other publications, which are incorporated in their entirety by reference herein; U.S. Pat. No. 4,336,173, U.S. Pat. No. 4,459,378, U.S. Pat. No. 4,654,267, U.S. Pat. No. 5,234,809, U.S. Pat. No. 5,705,628, U.S. Pat. No. 5,898,071, WO 96/09379, WO 99/58664, WO 02/055727, WO 05/089929, U.S. Pat. No. 5,648,124, WO 96/37313, U.S. Pat. No. 6,534,262, US 2003-0235839, US 2004-0214175, US2006-0003357, U.S. Pat. No. 6,812,341, U.S. Pat. No. 7,052,840, US 2006-0205004, WO 03/085091, US 2002-0106686, US 2006-0024701, US 2004-0215011, US 2006-0058519, US 2005-0239068, US 2005-0106577, US 2005-0106576, US 2005-0106589, US 2005-0106602, US 2005-0136477, WO 06/036243, WO 06/036246, WO 06/019387, WO 06/19388, WO 06/019568, US 2006-0177836, WO 06/015326, US2005-0027116, US 2002-0177698, U.S. Pat. No. 6,646,118, U.S. Pat. No. 6,429,309, WO 94/11103, WO 97/08547, WO 01/81566, WO 04/020449, U.S. Pat. No. 5,976,426, US 2005-0181378, US 2005-0196856, US 2006-0160122, US 2005-0271553, DeAngelis et al. “Solid-phase reversible immobilization for the isolation of PCR products” Nucleic Acids Research 23(22):4742-4743 (1995), Hawkins et al. “DNA purification and isolation using a solid-phase” Nucleic Acids Research 22(21):4543-4544 (1994), Aviv et al., “Purification of biologically active globin messenger RNA by chromatography on oligothymidlic acid cellulose,” Proc. Natl. Acad. Sci. USA, 69(6):1408-1412, 1972, Krizova et al., “Magnetic hydrophilic methacrylate-based polymer microspheres for genomic DNA isolation” J Chromatogr A. 2005 Feb. 4; 1064(2):247-53, Hirabayashi et al. “Applied slalom chromatography improved DNA separation by the use of columns developed for reversed-phase chromatography” J Chromatogr A. 722(1-2):135-42 (1996), Iuliano et al. “Rapid analysis of a plasmid by hydrophobic-interaction chromatography with a non-porous resin.” J. Chromatogr A. 972(1):77-86 (2002), Ausubel, et al., “Minipreps of Plasmid DNA,” Current Protocols in Molecular Biology 1998; Ch. 1: 1.6.1-1.6.10; Ch. 2:2.1.1-2.7.8, Meng et al., “Polyethylene glycol-grafted polystyrene particles” J. Biomed Mater Res A. 70(1):49-58 (2004), McFarland et al., “Separation of oligo-RNA by reverse-phase HPLC” Nucleic Acids Res. 7(4):1067-1080 (1979).

2. Modification of Solid Supports

The solid supports can be modified to impart different relative hydrophilic and hydrophobic properties to the matrix, which can be beneficial for recovering nucleic acids, proteins or other biomolecules. Such modifications are generally effected by the addition of a ligand to the solid support. Examples of ligands include, but are not limited to, organic compounds including alcohols, carboxylic acids, amines, esters, ethers, aromatic hydrocarbons, drugs, dyes and minor groove binders, hormones, amino acids, proteins, peptides, polypeptides, lectins, enzymes, enzyme substrates, enzyme inhibitors, cofactors, nucleotides, oligonucleotides (e.g., oligo dT), polynucleotides, carbohydrates, sugars and oligosaccharides. The imparted properties can be specific for a certain type of molecule, such as the modification of the support to specifically bind a polyA+ RNA by coating the support with oligo(dT), or the modification of the support to specifically bind an antigenic determinant by coating the solid support with an antibody. Alternatively, the modification can have a more general effect, such as, for example, modification to alter polarity (or charge) or hydrophobicity.

i. Modification of Charge/Hydrophilicity

Many materials used as the basis for solid supports are initially uncharged, including, but not limited to, polystyrene, polyethylene, polypropylene, polyvinylchloride, dextran and the like. For the purposes of isolating and purifying nucleic acid molecules or other biomolecules, the surface polarity or hydrophilicity of solid supports made of these and other materials can be adjusted to facilitate binding of the biomolecules to the solid support and/or their elution from the solid support. For example, by increasing the negative charge of the solid support, which also increases hydrophilicity, elution of nucleic acid molecules bound to a solid support can be enhanced. The presence of a charge on a solid support also can facilitate colloidal stability, reduce aggregation of the solid support particles, and provide maximum surface area for biomolecule, such as a nucleic acid, capture and binding.

Altering the surface charge of a non-charged material (e.g. polystyrene) can be achieved by using an initiator in the polymerization process that provides a charge. For example, a styrene monomer polymerized in the presence of a persulfate initiator will provide a polystyrene core having a negative surface charge, due to the negatively charged sulfate endgroups on the surface of the matrix arising from the decomposition of initiator molecules. Other examples of initiators that provide a negative charge include potassium peroxydiphosphate and 4,4′-azobis (4-cyanovaleric acid). In contrast, using 2,2′-(2-methylpropionamidine)dihydrochloride as the initiator will provide the polystyrene with a positive charge.

The surface charge of the solid support also can be modified by introducing a functional group such as a sulfhydryl group (SH) or a carboxylic acid group (COOH). For example, copolymerization of styrene monomers and acrylic or methacrylic acid using emulsion polymerization methods results in a polymeric matrix containing a free carboxylic acid group (Holzapfel V et al J. Phys Condens Matter 2006; 18: S2581-S2594; See also e.g., U.S. Pat. No. 5,648,124). Due to its polarity, this functional group imparts hydrophilic properties to the polymer, and is able to form hydrogen bonds with water molecules. The carboxylic acids partially ionize in water, forming COO⁻ groups on the surface of the solid support and increasing the negative charge. The negative surface charge can be increased by increasing the amount of acrylic acid in the polymerization process. Carboxylic acid groups also can be introduced via polymerization using an azoinitiator (e.g. 4,4′-azobis-4-cyanopentanoic acid (ACPA)), which itself provides carboxyl end groups, thereby negating the need for copolymerization with carboxylic acid monomers such as acrylic acid (Bastos D & de las Nieves F J Coll Polymer Sci 1996; 274(11):1435-1536).

Modification of the charge of a solid support by the introduction of carboxyl end groups also can be accomplished in the absence of a polymerization step. Solid supports with an adsorptively or covalently bound silane coat can be modified by coupling chemistries through the amino group of the amino silane on the particle. For example, a succinic acid moiety contains two carboxylic acid groups, one of which can bond with the amine through an amide bond, leaving the second group unbonded and tethered to the silane solid support. Alternatively, the amino group of the silane is reacted with glutaric anhydride to covert the terminal group from an amine to carboxylic acid, by first treating the silanized solid support with 0.1 M NaHCO₃, then reacting with glutaric anhydride (U.S. Pat. No. 4,695,393).

Modification of the charge of a solid support by way of carboxylation also can be effected by coating the solid support with a carboxylated layer. For example, a particle coating can be prepared using an epoxide compound having a functional group copolymerizable with an acrylate, e.g. a carbon-carbon double bond, for example using two or three epoxides, one of which contains an unsaturated carbon-carbon bond. The coated particles can then be functionalized by reaction with a vinyl or acrylic monomer carrying a functional group, such as a carboxylic acid group. Some commercially available beads, such as Dynabeads® M-270 Carboxylic acid (Dynal Biotech), employ this strategy to carboxylate paramagenetic beads. The polystyrene beads are crosslinked with magnetic material precipitated into the pores, and then coated with a layer of glycidal ether to encapsulate the iron oxide. Carboxylic acid groups are then introduced onto the surface by incubation with isopropanol, methanol, acrylic acid and 2,2′-azoisobutyronitrile (U.S. Pat. No. 6,986,913).

The surface charge of a solid support also can be modified by the inclusion of other surface groups. For example, sulphonate groups can provide a negative ionic charge, and can be incorporated into the polymer, such as in poly(styrene sulfonate), or dextran sulphate, by reaction with a sulfonic acid. Other anionic functional groups also can be used to modify the solid support and impart a negative charge, including boric, sulfinic, phosphoric, or phosphorus groups, or a combination thereof. Equally, it is understood that a solid support can be modified to possess a positive surface charge using methods well known to those of skill in the art, such as, for example, modification with cationic functional groups.

ii. Modification of Hydrophobicity

The underlying principles of many nucleic acid and protein purification techniques are based upon hydrophobic interactions. A protein or nucleic acid with hydrophobic groups on its surface can be purified based on hydrophobic interactions with an insoluble hydrophobic group immobilized on a solid support. Examples of hydrophobic ligands include, but are not limited to, uncharged ligands comprising long, optionally substituted, alkyl chains (e.g. butyl-, hexyl-, octyl-, decyl-, dodecyl-derived groups) and/or aromatic and heteroaromatic structures (e.g. phenyl-, naphthyl-, benzimidazole derived groups). Aliphatic compounds, in which the carbon atoms are joined together in straight or branched chains i.e. do not contain aromatic rings, increase in hydrophobicity as the carbon chain length increases, with least a 2-carbon chain being sufficient to create a hydrophobic region. The hydrophobic ligand also can contain one or more functional groups, such as for example, an amine, hydroxyl, aldehyde, carboxylate, ester, or thiol group.

Aliphatic Amines

Aliphatic amines can be considered ammonia derivatives in which one or more hydrogen atoms have been replaced by a hydrocarbon radical, and where the carbon atoms are linked in open chains. These hydrophobic molecules can be linked to a solid support by virtue of the amine functional group. For example, using carbodiimide coupling chemistry, an amide bond can be formed between a carboxylic acid group on the solid support and the amine group, thereby linking the hydrophobic aliphatic chain to the solid support. Aliphatic amines also can be coupled to, for example, free sulphonic acid groups to form a sulphonamide, an aldehyde group to form an imine, and esters to form an amide bond. Using these or similar coupling techniques, the hydrophobicity of the solid support can be modified, the degree of which is dictated by the level of coupling, and the length (and therefore, the relative hydrophobicity) of the carbon chain.

The carbon chain length of an aliphatic amine suitable for use in the modification of hydrophobicity can be any number generally in the range of from about or at 1 to about or at 3, 4, 5, 6, 7, 8, 9, 10, 11, 12, up to about or at 35, 40 or 45 carbon atoms. The carbon chain length of the amine generally, but not exclusively, can be about or at 2, e.g. ethylamine (C₂H₇N), and generally more, including, but not limited to, alkyl chains with 3 (propylamine), 4 (butylamine), 6 (hexylamine), 8 (octylamine), and more carbons, such as C₁₂, C₁₈ and C₂₄ alkyl chains. In some embodiments, the carbon chain length of the amine can be from about 3 carbon atoms to about 8 carbon atoms. Conceivably, any aliphatic amine with one, two, or more than two carbon atoms can be utilized to modify the hydrophobicity of a solid support. Other non-limiting examples of suitable aliphatic amines include butoxypropylamine, polypropylamine, HCl propylamine, 2-(2-aminoethoxy)ethanol, and aliphatic amines with additional moieties attached.

Aliphatic Amine-Polyethylene Glycols

Polyethylene glycols (PEGs) can be used advantageously to link enzymes or other functional entities to insoluble carriers and other biomolecules while retaining the activity of the entity (Stark M. and Holmberg K. Biotech. and Bioeng., 1989; 34:942-950). The use of the chemically-inert PEG spacer arms minimizes the steric effects caused by the support, and it's compatibility with a wide range of solvents makes it useful for most applications. Amine-PEG spacer molecules are commonly used to link a functional moiety, such as for example, a biotin label, an enzyme or a protein, to another molecule, and such linkers are commercially available. For example, Nektar Therapeutics and IRIS Biotech offer a range of monofunctional (one free reactive functional group) and heterofunctional (two free reactive functional groups) PEG linkers.

For purposes herein, the linker modifying the hydrophobicity of the beads can be an aliphatic amine linked to polyethylene glycols. In some embodiments, the polyethylene glycols can be linked to the amines without interfering with the ability of the amine group to form covalent bonds such as, for example, amide bonds through reaction of the amine functional group with a carboxylic acid. A heterofunctional PEG can be utilized so that an additional moiety can subsequently be readily attached to the PEG-aliphatic amine and, therefore, to a carboxylated solid support. A variety of PEGs can be linked to the aliphatic amine, including, but not limited to, hexaethylene glycol, heptaethylene glycol and decaethylene glycol. For example, exemplary aliphatic amine-polyethylene glycol ligands suitable for use in the methods described herein include a hexamine-hexaethylene glycol ligand and a hexamine-(hexaethylene glycol)₃ molecule.

Aliphatic Amines Linked to Nucleotides

The affinity of the aliphatic amine ligands for nucleic acid molecules can be adjusted by linking one or more nucleotides. Exemplary linkage is through a PEG moiety as described above in aliphatic amine-polyethylene glycols, although linkage also can be direct or through any other linking group that covalently links the nucleotide(s) to the ligand. The linked nucleotide(s) can conceivably be of any length and sequence. Sequence-specific polynucleotides can be attached to the aliphatic amine ligand that are complementary to a known nucleotide sequence, such as a conserved sequence in a gene or family of genes, or in a promoter or other response element. Alternatively, the polynucleotide can preferentially bind a species of nucleic acid. For example, poly-thymidines (or oligo (dT)s) can be used to preferentially bind and isolate mRNA species that contain a poly A tail. Most eukaryotic mRNAs (and some viral mRNAs) end in a poly A tail of between 20 and 250 adenosine nucleotides. The poly A tail provides a useful tool for selective isolation of this subset of nucleic acid molecules. Isolation of mRNA by binding through oligo(dT) primers has been widely reported (See e.g., U.S. Pat. Nos. 5,459,253 and 5,976,797) and many solid supports containing oligo (dT) molecules are commercially available for the selective isolation of mRNA. Poly-U sequences also can be used for this purpose. The length of oligo (dT) or poly-U sequences can be anything upward of 1, typically, 2, 5 10 or more nucleotide(s), although it is generally acknowledged that nucleic acid absorbance increases with the strand length of oligo dT (U.S. Pat. No. 5,508,166). Technologically no upper limit is imposed on length, and polynucleotide sequences of any length can be synthesized using techniques known to those of skill in the art.

Aliphatic Amines Linked to Minor Groove Binders

The hydrophobic aliphatic amine also can be linked to a minor groove binder (MGB) to enhance affinity for nucleic acid molecules. Exemplary linkage is through a PEG moiety as described above for aliphatic amine-polyethylene glycols, although linkage can be through any other linking group that covalently links the minor groove binder moiety to the ligand, such as for example, a stretch of six thymidines. Minor groove binders are a potent class of naturally occurring antibiotics that bind to duplex DNA specifically in the minor groove. Minor groove binders are long, flat molecules that can adopt a crescent shape that fits snugly into the minor groove to form close atomic contacts in the deep, narrow space formed between the two phosphate-sugar backbones in the double helix. They are stabilized in the minor groove by either hydrogen bonds or hydrophobic interactions. A molecule generally is considered to be a minor groove binder if it is capable of binding within the minor groove of double stranded DNA with an association constant of 10³ M⁻¹ or greater. This type of binding can be detected by well established spectrophotometric methods, such as ultraviolet (UV) and nuclear magnetic resonance (NMR) spectroscopy and also by gel electrophoresis. Shifts in UV spectra upon binding of a minor groove binder molecule, and NMR spectroscopy utilizing the “Nuclear Overhauser” (NOSEY) effect are particularly well known and useful techniques for this purpose. Gel electrophoresis detects binding of a minor groove binder to double stranded DNA or fragment thereof, because upon such binding the mobility of the double stranded DNA changes.

Minor groove binders have widely varying chemical structures, making it impossible to generate a general formula to describe them. The majority of the naturally-occurring MGBs described in the art have a strong preference for AT-rich regions of double-stranded B-DNA (the form of DNA in which the double helix twists in a right-hand direction) (Zimmer, C. & Wahnert, U. (1986) Prog. Biophys. Mol. Biol. 47, 31-112). However, synthetic minor groove binders that have a preference for GC-rich double-stranded DNA also have been generated (Forrows et al. (1995) Chemico-biological interactions. 96:125-142). A variety of suitable minor groove binders, and their derivatives, have been described in the literature (Kutyavin, et al. U.S. Pat. No. 5,801,155; Wemmer, D. E., and Dervan P. B., Current Opinon in Structural Biology, 7:355-361 (1997); Walker, W. L., Kopka, J. L. and Goodsell, D. S., Biopolymers, 44:323-334 (1997); Zimmer, C & Wahnert, U. Prog. Biophys. Molec. Bio. 47:31-112 (1986) and Reddy, B. S. P., Dondhi, S. M., and Lown, J. W., Pharmacol. Therap., 84:1-111 (1999). The oligonucleotide minor groove binding moiety conjugates show strong affinity to hybridize and strongly bind to complementary sequences of single or double stranded nucleic acids, and thereby have utility as sequence specific probes and as antisense and anti-gene therapeutic agents.

Compounds that are capable of binding in the minor groove of DNA, generally speaking, have a crescent shape three dimensional structure. Most minor groove binding compounds described in prior art have a strong preference for A-T (adenine and thymine) rich regions of the B form of double stranded DNA. Examples of known minor groove binding compounds of the prior art are certain naturally occurring compounds such as netropsin, distamycin and lexitropsin, mithramycin, chromomycin A₃, olivomycin, anthramycin, sibiromycin, as well as further related antibiotics and synthetic derivatives. Certain bisquartemary ammonium heterocyclic compounds, diarylamidines such as pentamidine, stilbamidine and berenil, CC-1065 and related pyrroloindole and indole polypeptides, Hoechst 33258, 4′-6-diamidino-2-phenylindole (DAPI) as well as a number of oligopeptides consisting of naturally occurring or synthetic amino acids are minor groove binder compounds.

The following are brief descriptions of non-limiting examples of minor groove binders. Distamycin A is an N-methylpyrrole-containing molecule originally isolated from Streptomyces distillicus. The tripyrrole peptide is characterized by the presence of an oligopeptidic pyrrolcarbamoyl frame ending with an amidino moiety, which reversibly binds to the minor groove of the DNA either in a monomeric or a side-by-side dimeric binding mode, by hydrogen bonds, van der Waals contacts, and electrostatic interactions with strong preferences for AT-rich sequences containing at least four AT base pairs (Chen, X. et al. (1994) Nat. Struct. Biol. 1, 169-175). Homologs, such as tetra-, penta-, and hexa-methylepyrrolecarboxamides also have been synthetically constructed (Youngquist R S., & Dervan P B. (1985) PNAS 82:2565-2569).

Hoechst 33258 is an antibiotic, but most commonly used as a DNA fluorochrome. It is a molecule that can be schematisized as phenol-benzimidazole-benzimidazole-piperazine, in which the NH groups of the benzimidazoles make bridging three-center hydrogen bonds between adenine N-3 and thymine 0-2 atoms on the edges of base-pairs. Steric clash between the drug and DNA dictates that the phenol-benzimidazole-benzimidazole portion of Hoechst 33258 binds only to AT-rich regions of DNA (Pjura P E. et al. (1987) J Mol Biol 197(2):254-271).

Netropsin is a naturally-occurring MGB from Streptomyces netropsis. It can be regarded as being assembled with a guanidinium, amide, methylpyrrole, amide, methylpyrrole, amide and propylamine, and binds within the minor groove by displacing the water molecules of the spine of hydration (Kopka M L., et al. (1985) PNAS 82:1376-1380).

Furamidine (DB75) and its related compounds bind as monomers to AT-rich sequences of DNA. However, an unsymmertic derivative, DB293, in which one of the phenyl rings of furamidine is replaced with a benzimidazole, binds to GC-containing sites on DNA more strongly that to pure AT sequences (Wang L et al. (2000) PNAS 97(1):12-16).

Examples of other minor groove binding compounds include, but are not limited to, naturally occurring compounds such as lexitropsin, mithramycin, chromomycin A₃, olicomycin, anthramycin, sibiromycin, and their derivative. Certain bisquanterary ammonium hereocyclic compounds, diarylamides such as pentamidine, stilbamidine and berenil, CC-1065 and related pyrroloindole and indole polypeptides, DAPI, as well as a number of oligopeptides consisting of naturally-occurring or synthetic amino acids also are minor groove binders.

Other exemplary minor groove binders are those selected from the formulae:

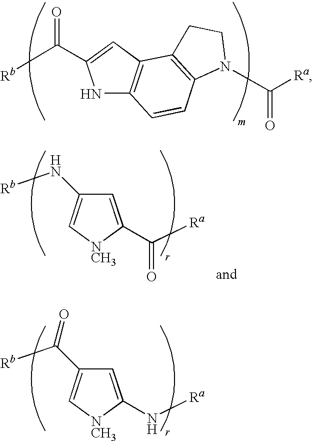

wherein the subscript m is an integer of from 2 to 5; the subscript r is an integer of from 2 to 10; and each R^{a} and R^{b} is independently a linking group to the oligonucleotide (either directly or indirectly through a fluorophore), H, —OR^{c}, —NR^{c}R^{d}, —COOR^{c} or —CONR^{c}R^{d}, wherein each R^{c} and R^{d} is selected from H, (C₁-C₁₂)heteroalkyl, (C₂-C₁₂)heteroalkenyl, (C₂-C₁₂)heteroalkynyl, (C₁-C₁₂)alkyl, (C₂-C₁₂)alkenyl, (C₂-C₁₂)alkynyl, aryl(C₁-C₁₂)alkyl and aryl, with the proviso that one of R^{a} and R^{b} represents a linking group to ODN or Fl. Each of the rings can be substituted with on or more substituents selected from H, halogen, (C₁-C₈)alkyl, OR^{g}, N(R^{g})₂, N⁺(R^{g})₃, SR^{g}, COR^{g}, CO₂R^{g}, CON(R^{g})₂, (CH₂)₀₋₆SO₃—, (CH₂)₀₋₆CO₂⁻, (CH₂)₀₋₆OPO₃⁻², and NHC(O)(CH₂)₀₋₆CO₂⁻, and esters and salts thereof, wherein each R^{g} is independently H or (C₁-C₈)alkyl. Particular examples have the structures shown below:

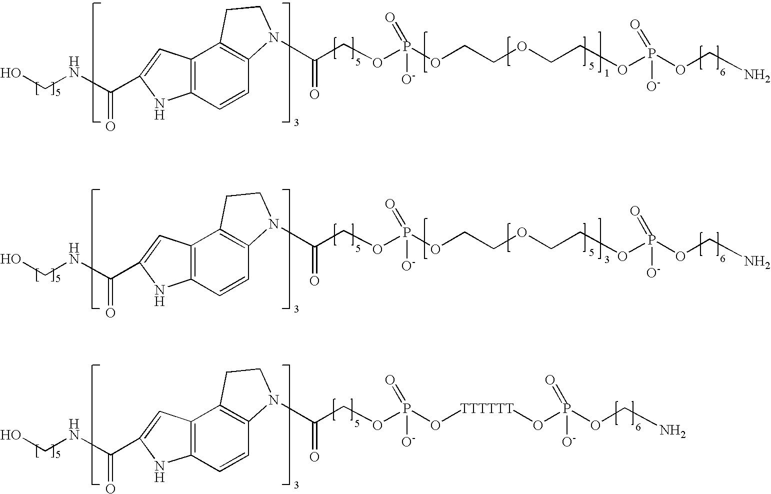

Other minor groove binders include the trimer of 1,2-dihydro-(3H)-pyrrolo[3,2-e]indole-7-carboxamide (CDPI₃), the pentamer of N-methylpyrrole-4-carbox-2-amide (MPC₅) and other minor groove binders that exhibit increased mismatch discrimination. Additional MB moieties are well known (see, e.g., U.S. Pat. Nos. 5,801,155; 6,084,102; 6,312,894 and 6,727,356. In certain embodiments, the MBs can have attached water solubility-enhancing groups (e.g., sugars, amino acids, carboxylic acid and/or sulfonic acid substituents,).

iii. Modification of Solid Supports with Hydrophilic and Hydrophobic Ligands

For the purposes of the methods described herein, when the ligand is a minor groove binder (MGB), the minor groove binder molecule is derivatized to facilitate linkage to an appropriate covalent structure or chain of atoms that attaches it to the modifying ligand, and thus to the solid support. The derivatized form of the MGB, which for purposes herein is a “radical,” is herein referred to as a minor groove binder moiety. The linking group is a moiety that covalently links the minor groove binder moiety to the ligand, such as for example, the hydrophobic aliphatic alkyl chain. Typically, the linking group is such that the linkage occurs through a chain of no more than about 20, typically 15, 16, 17 or 18 atoms. In some embodiments, the MGB is covalently attached to the end of a hydrophobic aliphatic alkyl chain, or to groups such as hexaethylene glycol. Attachment is to any position of the ligand that does not interfere with the ability of the ligand to couple to the solid support.

Generally for modification of solid supports with ligands, the linking group is derived from a bifunctional molecule so that one functionality such as an amine functionality is attached to a carbonyl group (CO) on the ligand, and the other functionality such as a carbonyl group (CO) is attached to an amino group of the minor groove binder moiety. As an exemplary alternative, the linking group can be derived from an amino alcohol so that the alcohol function is linked to the ligand and the amino function is linked to a carbonyl group of the minor groove binder moiety.

The modifying ligand can be attached to the solid support, for example, to a paramagnetic bead, through a reactive group on the support. For example, the polymeric coating of a bead or other support can be derived from one, or more than one, polymer that contains a reactive group which, when activated, chemically bonds the polymer molecule containing the reactive groups to an appropriate ligand. Reactive groups include, but are not limited to, carboxylic acid groups, epoxides, oxiranes, N-hydroxysuccinimide, aldehydes, hydrazines, maleimides, mercaptans, amines, alkylhalides, isothiocyanates, carbodiimides, diazo compounds, tresyl chloride, tosyl chloride, and trichloro-S-triazine, esters, ketones, anhydrides, mixed anhydrides, acyl halides, hydrazines, benzimidates, nitrenes, isothiocyanates, azides, sulfonamides, bromoacetamides, iodocetamides, sulfonylchlorides, hydroxides, thioglycols, or any reactive group known in the art as useful for forming conjugates. Alternatively, the solid support can contain an appropriate functional group that can be coupled to a complementary functional group on a ligand using a coupling agent. If an appropriate reactive or functional group is not already embedded in the solid support, the surface can be modified to contain such groups. For example, a polystyrene-encapsulated paramagnetic bead can be carboxylated using methods well known in the art. Other materials that can be readily carboxylated include, but are not limited to, polypropylene, nylon, glass, polyethylene, polycarbonate and silicon.

A carboxylated support can be coupled to ligands that contain an amine group, by using coupling agents that initiate the formation of a covalent amide bond. Carbodiimides, such as for example, DCC (acronym for N,N′-dicyclohexylcarbodiimide), DIC (acronym for N,N′-diisopropylcarbodiimide) and EDAC HCl (acronym for 1-ethyl-3-(3-dimethylaminopropyl) carbodiimide hydrochloride), couple carboxyls to primary amines, resulting in the formation of an amide bond. The carbodiimides react with the carboxylic acid group and activate the carboxyl group to form an active O-acylisourea intermediate, which can be viewed as a carboxylic ester with an activated leaving group. The O-acylisourea will react with the amine groups to generate the amide bond and urea.

Coupling of a carboxylated paramagnetic bead, or other suitable solid support, to an amine ligand using the carbodiimide coupling method requires specific reaction conditions. The hydrolysis of EDAC is a competing reaction during coupling and is dependent on temperature, pH and buffer composition. 4-Morpholinoethanesulfonic acid (MES) and imidazole are non-limiting examples of effective carbodiimide reaction buffers. Phosphate buffers reduce the reaction efficiency of the EDAC, but increasing the amount of EDAC can compensate for the reduced efficiency. In contrast, Tris, glycine and acetate buffers typically are not suitable carbodiimide coupling buffers. The coupling reaction can be performed between pH 4.5 to 5 and requires only a few minutes for many applications. However, the yield of the reaction is similar at pH from 4.5 to 7.5. Ligand concentrations also are important in the coupling reaction, and can be varied depending upon the desired degree of coupling. Saturation of the carboxyl groups with ligand, with the appropriate amounts of coupling reagent and buffer, generally results in a high degree of coupling, while a low ligand to carboxyl ratio typically results in a lower degree of coupling.

### C. Nucleic Acid Isolation Using Modified Solid Supports

The modified solid supports provided herein can be used to isolate, i.e., purify or separate and recover nucleic acids, proteins or other biomolecules. Solid phase isolation of nucleic acids can be applied to nucleic acids of essentially any length. Short fragments, such as for example, oligonucleotides of between 5 and 50 nucleotides in length, are routinely isolated using solid supports. Equally so are larger nucleic acid molecules, such as those that make up plasmids and generally range from 1 to over 400 kilobases (kb). Nucleic acid molecules of even greater length, such as those that compose the genome of organisms and exceed 3 billion nucleotides, also can be isolated by methods that involve binding to solid supports. The size of the nucleic acid molecule isolated using the methods described herein also can be of any length intermediate to the extremes noted above.

1. Types of Nucleic Acids and Samples Containing Nucleic Acids

Nucleic acids can be in the form of deoxyribonucleic acid (DNA), ribonucleic acid (RNA) or peptide nucleic acid (PNA), as well as analogs, derivatives or any combination thereof. Such a derivative could contain, for example, a nucleotide analog or a “backbone” bond other than a phosphodiester bond, for example, a phosphotriester bond, a phosphoramidate bond, a phosphorothioate bond, or a thioester bond. Naturally-occurring RNA molecules include, but are not limited to, transfer RNA (tRNA; generally smaller molecules of approximately 75 nucleotides), ribosomal RNA (rRNA; ranging from approximately 100 to 3000 nucleotides), messenger RNA (mRNA; of variable length), or genomic RNA, such as that from influenza or hepatitis C viruses. Other forms of RNA include, but are not limited to, small interfering RNA (siRNA; typically 100 nucleotides or fewer) and microRNA (miRNA). The nucleic acid molecules can be single-stranded (ss; and which can be sense or antisense), double-stranded (ds) or a combination of the two, and can be linear or circular, the latter of which can be open-circular or closed-circular. Covalently closed circular double-stranded DNA molecules also can supercoiled, referring to a higher order tertiary structure. Double-stranded DNA molecules can adopt several helical conformations, characterized by the direction of the helical turn, the helix diameter, the number of base pairs per helical turn and other parameters. A-DNA has a right-handed helix with approximately 11 base pairs per turn and a diameter of 25.5 A; B-DNA has a right-handed helix with approximately 10 base pairs per turn and a diameter of 23.7 A; and Z-DNA has a left-handed helix with approximately 12 base pairs per turn and a diameter of 18.4 A.

A nucleic acid used in the isolation methods described herein can be naturally-occurring or made by any technique known to those of skill in the art, such as for example, chemical synthesis or recombinant production. Non-limiting examples of a synthetic nucleic acid (e.g. a synthetic oligonucleotide) include a nucleic acid made by in vitro chemical synthesis using phosphotriester, phosphite or phosphoramidite chemistry and solid phase techniques (see, e.g. EP 266032) or via deoxynucleoside H-phosphonate intermediates (see, e.g. U.S. Pat. No. 5,705,629 and Froehler B C et al Nucleic Acids Res. 1986 Jul. 11; 14(13): 5399-5407). Enzymatically-produced nucleic acids can include, but are not limited to, those produced by polymerases in amplification reactions such as PCR, the enzymatic synthesis of oligonucleotides described in U.S. Pat. No. 5,645,897, and the creation of synthetic RNAs using ligase (Stark M R et al RNA. 2006 Sep. 18; Epub ahead of print). Still further, nucleic acids used in the isolation methods described herein can be modified. Examples of modifications include, but are not limited to, the inclusion of fluorescent moieties to the 5′ or 3′ ends or internally, 3′-aminopropyl modification (or 3′-terminal capping) such as that commonly used in antisense oligonucleotide synthesis, biotinylation, and modification of the normal phosphodiester backbone, such as by the inclusion of methyl phosphonates. Nucleic acids can be modified to facilitate detection by including a detectable label such as a fluorescent, radioactive, luminescent or chemiluminescent label. A nucleic acid molecule also can contain one or more backbone bonds that are selectively cleavable, for example, chemically, enzymatically or photolytically. For example, a nucleic acid molecule can include one or more deoxyribonucleotides, followed by one or more ribonucleotides, which can be followed by one or more deoxyribonucleotides, such a sequence being cleavable at the ribonucleotide sequence by base hydrolysis. A nucleic acid molecule also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. Nucleic acids Res. 25: 2792-2799 (1997)).

Nucleic acids can be exogenous, meaning the nucleic acid originated outside a host organism and has been introduced into the host organism. This type of nucleic acid is often produced by recombinant means, such as by the cloning of a fragment of DNA into a plasmid, the introduction of the plasmid into a host cell, and the replication of the recombinant DNA vector by the host cell. Recombinant molecules that can be introduced into a host cell include, but are not limited to, bacterial artificial chromososmes (BACs), yeast artificial chromosomes (YACs), P1-derived artificial chromosomes (PACs), cosmids and plasmids. Other appropriate host cells include, but are not limited to, yeast cells, plant cells and mammalian cells. The exogenous nucleic acid can be introduced into the host cell, or an ancestor thereof, by methods well known to those of skill in the art, such as transfection or transformation methods. Alternatively, the nucleic acid can be indirectly introduced into the host cell, or its ancestor, by use of a phage.

A nucleic acid molecule also can be endogenous, meaning it exists naturally in the organism. The nucleic acid can be endogenous to any prokaryotic or eukaryotic cell, virus, bacteriophage, mycoplasma, protoplast or organelle.

Nucleic acid molecules can be isolated from any sample containing them. The sample can be a relatively pure sample, such as the product of a PCR or restriction enzyme digestion, or an agarose solution containing nucleic acid molecules. The sample also can be a semi-pure preparation obtained by other nucleic acid recovery processes, such as for example, a phenol/chloroform extraction typical of those methods well known to those of skill in the art. Such samples contain free or ‘naked’ nucleic acids. The sample can be a clinical or environmental sample, or can be food and allied products, or can be the products of an oligonucleotide synthesis reaction. Often, the sample will be a biological material, which can include any viral or cellular material, including prokaryotic or eukaryotic cells, viruses, bacteriophages, mycoplasmas, protoplasts and organelles. The biological materials include all types of mammalian and non-mammalian animal cells, plant cells, algae, fungi, bacteria and protozoa. Representative samples therefore include, but are not limited to, whole blood and blood-derived products such as plasma, serum and buffy coat, urine, feces, nasopharyngeal specimens, cerebrospinal fluid or any other body fluid, tissues, cell cultures, cell suspensions and cell lysates.

2. Methods of Isolating Nucleic Acids Using Solid Supports

The isolation of nucleic acids is a necessary step for a multitude of applications in the fields of, for example, molecular biology, biotechnology and medicine, and is required for both diagnostic, therapeutic and research purposes. In most instances, the purity and quality of the isolated nucleic acid, the recovery efficiency, and the ease with which the nucleic acid is isolated, are all equally important. Nucleic acid isolation has evolved from a multi-step process involving organic chemicals to simpler methods using solid supports and fewer steps. By virtue of various chemical and/or physical interactions, the nucleic acids in a given solution bind to the solid support while the remainder of the solution and its components are washed away. The bonds between the nucleic acid and the solid support are reversible however, and can be broken, generally by changing the microenvironment, such as by decreasing the pH or ionic strength, and the isolated nucleic acid is eluted, free from unwanted or extraneous material and chemicals.

A bead, or other solid support, with or without ligand modification, or using the solid supports modified with hydrophilic and hydrophobic ligands to adjust their relative hydrophilic and hydrophobic surfaces for nucleic acid capture and elution, can be used to adsorb nucleic acid under the appropriate conditions. Such conditions are achieved by the use of appropriate buffers, which are modified by, for example, altering pH or salt concentrations, and including variously functional agents, to alter the adsorption characteristics.

a. Binding

A sufficient quantity of a nucleic acid precipitation agent is required to adsorb the nucleic acid onto the suspended beads. Precipitation and binding of nucleic acid to solid supports can be effected by an agent such as, for example, a polyalcohol including polyethylene glycol (PEG), in the presence of high salt concentration. The molecular weight of the PEG can range from about 6,000 to about 10,000, from about 6,000 to about 8,000, from about 7,000 to about 9,000, from about 8,000 to about 10,000. In general, any molecule, the presence of which, like PEG, provides an environment that forces hydrophilic nucleic acid molecules out of solution, can be used. Such a strategy has been reported widely in the literature. For example, a binding buffer containing 20% PEG 8000 and 2.5 M NaCl can be used to adsorb double stranded plasmid DNA and single stranded bacteriophage DNA to magnetic microparticles (U.S. Pat. No. 5,705,628; U.S. Pat. No. 5,898,071; Hawkins T L et al. Nucleic Acids Res 1994; 22:4543-4544), while in another example, PEG 8000 concentrations ranging from 11% to 40% can be used in conjunction with variable concentrations of salt (e.g. 0.6 M to 3.3 M NaCl, or 20 mM MgCl₂) to precipitate different sized DNA molecules onto paramagnetic microparticles (U.S. Pat. No. 6,534,262). Other examples of the use of PEG-salt buffers for the precipitation of nucleic acid onto solid supports include, but are not limited to, those described in US 2002/0106686, US 2006/0024701, WO 97/08547 and DeAngelis et al. (Nucleic Acids Res 23:4742-4544). Salts other than NaCl also can be included in the buffer to facilitate the adsorption of the nucleic acid to the solid phase carrier. These include lithium chloride (LiCl), barium chloride (BaCl₂), potassium (KCl), calcium chloride (CaCl₂), magnesium chloride (MgCl₂) and cesium chloride (CsCl). Often, the presence of salt functions to minimize the negative charge repulsion of the nucleic acid molecules.

Chaotropic agents, which are those that alter the secondary, tertiary, and/or quanternary structure of proteins and nucleic acids, but leave the primary structure intact, also can be used in buffers to precipitate the nucleic acid and facilitate adsorption to the beads. Non-limiting examples of chaotropic compounds useful for precipitating nucleic acid are guanidinium chloride, guanidinium thiocyanate, guanidinium isothiocyanate, sodium thiocyanate, sodium iodide, potassium iodide and urea. High concentrations typically are required for efficient nucleic acid precipitation, such as for example, 1M, 2M, 3M and 4M guanidinium thiocyanate. Chaotropic substances such as guanidinium thiocyanate function not only to precipitate the nucleic acid, but also to denature other proteins in the starting material. In doing so, a chaotropic buffer also can function as a lysis buffer, whereby lysis of any cells, viruses, or associated matrices or packaging, is initiated to release all of the nucleic acid present in the starting material and precipitate it onto the beads.

Other denaturants, or detergents, also can be included in the buffer to aid extraction and subsequent precipitation of nucleic acids from such starting material. The detergent can act to solubilize the sample. Detergents can be ionic or nonionic. Examples of nonionic detergents include Triton, such as the Triton X series (Triton X-100, Triton X-100R, Triton X-114, Triton X-450, Triton X-450R), octyl glucoside, polyoxyethylene(9)dodecyl ether, digitonin, IGEPAL CA630, n-octyl-beta-D-glucopyranoside (betaOG), n-dodecyl-beta, C12EO7, Tween 20, Tween 80, polidocanol, n-dodecyl beta-D-maltoside (DDM), NP-40, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent also can be used in the purification schemes. These include, for example, Chaps, zwitterion 3-14, and 3-[(3-cholamidopropyl)dimethylammonio]-1-propanesulfonate.

Lysis or homogenization solutions further can contain other agents such as reducing agents. Examples of such reducing agents include dithiothreitol (DTT), beta-mercaptoethanol, dithioerythritol (DTE), glutathione (GSH), cysteine, cysteamine, tricarboxyethyl phosphine (TCEP), or salts of sulfurous acid. Optionally, lysozyme also could be included in the lysis component of the binding buffer.

In one example, a suitable chaotropic lysis buffer for adsorbing nucleic acids from complex starting materials, such as serum, blood, feces, or urine, to silicon dioxide particles is one that contains 8 M guanidinium isothiocyanate, 0.8 M Tris HCl, 0.03 M EDTA and 2% Triton X-100. The guanidinium isothiocyanate can be substituted with another chaotropic substance, such as potassium iodide (3M), sodium iodide (3 M), sodium thiocyanate (3 M), or any of these in combination with 8M urea. In addition to facilitating the absorption of nucleic acid molecules to silicon dioxide particles, these binding buffers can be used in conjunction with other solid supports, such as silica derivatives, polystyrene latex particles, and PVDF or nitrocellulose filters (U.S. Pat. No. 5,234,809). A multistep method also can be used to bind nucleic acid to a solid support with chaotropic buffers. RNA can be isolated from frozen or fresh tissue by initially homogenizing the tissue in a buffer containing 4 M guanidine thiocyanate, 0.1 M betamercaptoethanol, 0.5% N-lauroyl sarcosine and 25 mM Na-citrate, pH 7.2. The subsequent addition of absolute ethanol to a final concentration of between 35 and 70% facilitates RNA adsorption when the solution is passed over glass fiber filter column (US 2005/0059024). As understood by those of skill in the art, the relative concentrations of the chaotropic agent, alcohol, and other buffer constituents can be altered, within limits, without adverse affects (See e.g. US 2005/0059024).

Many other buffers can be developed that facilitate adsorption of nucleic acid molecules to a solid support. For example, glycogen can be included as the precipitating agent in an ammonium acetate-based buffer to precipitate RNA on to carboxylated beads (U.S. Pat. No. 7,052,840). In one embodiment, 200 μl of mouse liver RNA is mixed with 100 μl 10 M ammonium acetate and 60 μg glycogen to facilitate adsorption to the beads. Chloride salts, such as sodium chloride, lithium chloride, magnesium chloride and potassium chloride, can be substituted for the ammonium acetate (or other acetate salts), and a range of glycogen concentrations (e.g. 100 μg/ml to 600 μg/ml) can be used effectively.

The composition of the binding buffers is determined based upon the type of nucleic acid to be isolated, and the solid support used in the isolation. While some binding buffers are suitable for a variety of purposes (e.g. PEG-salt buffers and chaotropic buffers), others can be specifically modified for a given method. For example, when using poly(dT) beads to bind mRNA species, hybridization of the polyA tail of the mRNA and the T residues on the bead can be achieved using approximately 0.5 M KCl, NaCl or LiCl in 10 mM Tris pH 7.5. Modifications can be made to include isostabilizing salts such as tetramethylammonium (TMA⁺), tetraethylammonium (TEA⁺) and betaine. These function to equalize the hydrogen bonding strength of the G-C and A-T pairs, thereby reducing the hybridization of contaminating rRNA and mRNA that might normally display advantageous G-C hybridization (U.S. Pat. No. 6,812,341).

b. Separating the Solid Support from Solution

Once the nucleic acid has been bound to the solid support, the solution in which the nucleic acid was originally contained is removed. The means by which this is achieved depends upon the nature of the solid support. When the support is a column, the solution is merely allowed to drain from the support by gravity flow. In cases where the nucleic acid is bound to a solid support such as a microtiter well, or flat plate, filter or membrane, the solution can be extracted by pipette, vacuum, or other physical means such as shaking. When particles, microparticles or beads are being used as the solid support, they can be concentrated and collected, for example at the bottom of a tube, to facilitate removal of the solution by pipette or vacuum suction. When the beads or particles are large, this can be achieved by allowing the tube to sit without agitation and letting the particles settle by gravity to the bottom. If the beads or particles are too small for gravity to be an efficient means of collection, then the tube can be centrifuged to force the particles to the bottom. For example, 1 to 5 μm diameter silica beads, contained in a 1.5 ml tube, can be centrifuged for 15 seconds at 12000×g. The supernatant can be removed from the bead pellet by vacuum suction or manual pipette. Still further, the particles or beads can be magnetically responsive, enabling collection of the beads via a magnetic force. For example, the paramagnetic beads can be contained in a test tube to which an external magnetic field is applied by way of an embedded rare earth (e.g. neodymium) magnet. The paramagnetic beads are attracted to the magnet and concentrated, facilitating removal by, for example, pipetting or vacuum suction, of any solution from the tube and the beads. The magnet is then removed from the vicinity to remove the magnetic force, and the beads fall to the bottom of the tube.

C. Washing

Optionally, a “wash buffer” can be employed prior to elution when isolating nucleic acid molecules using paramagnetic beads, or other appropriate solid supports. The wash buffer functions to remove impurities (e.g. host cell components, proteins, metabolites or cellular debris) that are bound either directly to the bead, or to the adsorbed nucleic acid molecules. The composition of the wash buffer is chosen to ensure that impurities are dissolved and removed. The pH and solute composition and concentration of the wash buffer can be varied according to the types of impurities that are expected to be present, and the binding forces between the nucleic acid and the solid support. For example, ethanol exemplifies a wash buffer useful to remove excess detergent and salt. The solid phase carrier with bound DNA also can be washed with more than one wash buffer solution. The solid phase carrier can be washed as often as required (e.g., three to five times) to remove the desired impurities. The number of washings generally is limited to minimize loss of yield of the bound DNA.

A suitable wash buffer solution has several characteristics. First, the wash buffer solution must have a sufficiently high salt concentration (i.e. be of sufficiently high ionic strength) that the nucleic acid bound to the solid phase carrier does not elute off of the solid phase carrier, but remains bound. A suitable salt concentration is greater than about 0.2 M, but can be reduced when stronger forces bind the nucleic acid to the solid support. For example, a 10 mM Tris buffer, pH 8.0 can be used to wash nucleic acid bound to a solid support that contains multiple nucleic acid binding groups, and which resists elution under most commonly-used elution conditions (US 2005/0106589; US 2005/0106602). Alternatively, the wash buffer can have a sufficiently high alcohol content, such as ethanol, to ensure that the nucleic acid remains a precipitate attached to the solid support. For example, DNA bound to paramagnetic particles using a PEG-salt binding buffer can be washed in a solution containing 70% EtOH and 10 mM EDTA (U.S. Pat. No. 6,534,262). Second, the wash buffer solution is chosen so that impurities that are bound to the DNA or solid phase carrier are dissolved. The pH and solute composition and concentration of the buffer solution can be varied according to the types of impurities which are expected to be present. For example, a suitable non-limiting set of wash buffers useful in the isolation of nucleic acid from viral particles includes; (I) 1.67 M guanidinium isothiocyanate, 33% isopropyl alcohol, 0.33% lauroylsarcosine, 0.033 M Tris HCl, pH 7.0; and (II) 70% ethanol, 10 mM KCl, 2 mM Tris pH 7.0, 0.2 mM EDTA, pH 8.0.

Many wash buffers suitable for various situations have been described and can be modified by those of skill in the art to optimize the conditions whilst retaining the essential qualities. For example, 100 mM ammonium sulphate, 400 mM Tris pH 9, 25 mM MgCl₂ and 1% bovine serum albumin (BSA), or 25 mM Tris acetate pH 7.8, 100 mM potassium acetate, 10 mM magnesium acetate and 1 mM dithiothreitol are suitable wash buffers when DNA is bound to paramagnetic particles using a PEG-salt binding buffer (U.S. Pat. No. 5,898,071; US 2002/0106686). Wash buffers can be of similar constitution to the binding buffer. For example, consecutive washes with 2 M TMA⁺ and 0.2 M TMA⁺ can be used when mRNA is bound to poly(dT) beads with a 4 M TMA⁺ binding buffer (U.S. Pat. No. 6,812,341). Similarly, tissue RNA bound to a glass fiber filter column using a buffer containing 4 M guanidinium thiocyanate is washed first with 4M guanidinium thiocyanate in 70% ethanol, then 80% ethanol, 0.1 M NaCl, 4.5 mM EDTA, 10 mM Tris HCl, pH 7.5 (US 2005/0059024). When a PEG-based binding buffer with a high ionic strength (i.e 2.5M NaCl) is used to bind DNA to magnetic beads, the beads can be washed first with 5 M NaCl, then 25 mM Tris acetate pH 7.8, 100 mM potassium acetate, 10 mM magnesium acetate and 1 mM dithiothreitol U.S. Pat. No. 5,705,628).

d. Elution

As discussed above, low concentrations of salt, such as for example, less than 0.2 M, results in significantly reduced binding of nucleic acid to the solid support in many protocols. Solutions containing a low concentration of salt can often therefore be utilized as elution buffers, which act to release or elute the bound nucleic acid molecules from beads, or other solid support. Aqueous elution buffers suitable for the dissociation of nucleic acid molecules from solid supports are known. These, include, but are not limited to, TE buffer (typically 10 mM Tris, 1 mM EDTA pH 7.5 to 8.0; U.S. Pat. No. 7,052,840), 0.1×TE pH 7.5-8.0, Tris-HCl (10 mM), EDTA (e.g. 0.1 mM pH 8.0; US 2005/0059024), Tris acetate (DeAngleis M M et al. Nucleic Acids Res 1994; 23:4742-4743), potassium chloride buffer (1 mM KCl, 0.2 mM sodium citrate), sucrose (e.g. 20%), formamide (e.g. 70% or 100%; U.S. Pat. No. 6,534,262), formamide/EDTA (e.g. 70%/1 mM; see, e.g., U.S. Pat. No. 6,534,262), pyrrolidinone (e.g. 12%; U.S. Pat. No. 6,534,262) and nuclease-free water (see, e.g., U.S. Pat. Nos. 5,705,628, U.S. Pat. Nos. 5,898,071 and 6,534,262, published U.S. application No. 2005/0196856). Other elution buffers known in the art include, but are not limited to, 1 mM sodium citrate pH 6.4, which optionally can be pre-warmed, and is used to elute mRNA from poly(dT) beads (see e.g., U.S. Pat. No. 6,812,341). Other elution buffers also can be developed to suit particular binding conditions. For example, nucleic acid bound to high affinity to beads containing multiple nucleic acid binding groups can be eluted with buffers that contain an organic solvent, such as 5% DTT and salt, such as 0.75M NaCl (US 2005/0106589). The selection of the elution buffer also is co-determined by the contemplated use of the isolated nucleic acid. For example, if the isolated nucleic acid is to immediately be used in an enzymatic reaction such as PCR, then a buffer with little or no EDTA should be used, as EDTA interferes with the function of many enzymes by binding the metal ions required for their activity.

### D. Nucleic Acid Separation Using Modified Solid Supports

Modifications can be made to a basic isolation protocol that enables the user to separate various nucleic acid species on a solid support. Separation can be based on the type of nucleic acid e.g. DNA versus RNA, or the molecular size of the nucleic acid molecule e.g. 100 nt versus 1000 nt. Separation can be effected by altering the properties of the solid support, the binding conditions, wash conditions, elution conditions, or any combination thereof.

Separation of nucleic acid molecules has been particularly effective in the field of chromatography, and different technology platforms are routinely used. For example, ion-exchange chromatography separates nucleic acid on the basis of charge, size exclusion chromatography separates nucleic acid on the basis of size, while HIC (hydrophobic-interaction chromatography) separates nucleic acid molecules on the basis of hydrophobicity. Anion-exchange chromatography uses a resin carrying positively charged groups (e.g. diethyl aminoethyl (DEAE)) that adsorb negatively charged molecules (e.g. negatively charged phosphates of the DNA backbone) in buffers near neutral pH and of medium ionic strength. The specific physical properties of the resin, combined with selected buffering conditions, such as pH and salt concentration, determine the selectivity potential. For example, an anion-exchange resin with dense coupling of the DEAE groups on the silica beads has a very high charge density. As such, there is selective binding of plasmid DNA from, for example, cell lysate until elution with a high-salt buffer, while impurities such as RNA, protein, carbohydrates, and small metabolites are washed from the resin with medium-salt buffers. At neutral pH, dNTPs can be eluted in a buffer with approximately 0.1 M NaCl, 30mer oligos at approximately 0.5M NaCl, tRNA at approximately 0.8 M NaCl, rRNA at approximately 0.9, M13 ssRNA at approximately 1.3 M NaCl, and plasmid and genomic DNA at approximately 1.5 M NaCl. If a relatively pure sample of DNA is first obtained (i.e. without RNA and cellular proteins), then anion-exchange chromatography can be used to effectively separate and purify DNA molecules of various sizes by simply altering the salt concentration of the elution buffer. Similarly salt-dependent elution and separation is exhibited using solid supports with alternative charged groups, such as an acrylic acid amide (US 2003/0171443). The type of resin used can be selected to preferentially bind nucleic acids with particular properties. For example, use of a weak anion-exchange material facilitates the selective isolation and purification of oligonucleotides that contain less than 200 bases or base pairs. (WO 97/29825). Many other examples exist in the art of anion-exchange chromatography in the isolation and/or separation of nucleic acids, many of which exhibit specific properties to influence their selective binding potential (e.g. U.S. Pat. No. 5,856,192, U.S. Pat. No. 5,660,984, U.S. Pat. No. 6,310,199, US 2004/0016702 and Merion M. & Warren W. Biotechniques 1989; 7:60)

HIC can be conducted to resolve molecules based on differences in their surface hydrophobicity. Interactions between hydrophobic groups with hydrophobic ligands attached to a chromatographic matrix mediate this chemistry. The type of matrix, the nature of the hydrophobic groups, and the conditions of absorption and elution can be tailored to suit the unique properties of the molecules involved. While originally developed to separate proteins, this technology can be used to separate nucleic acids, particularly to separate native, double-stranded supercoiled plasmid DNA from more hydrophobic nucleic acids, such as RNA, denatured gDNA and oligonucleotides (Diogo et al. Biotechnol Bioeng 2000 68: 576-583), or double-stranded supercoiled plasmid DNA from nicked open-circular plasmid DNA (Iuliano S et al. J Chromatog A 2002; 972:77-86, Prazares D J Chromatog A 1998; 806:31). HIC is generally practiced by binding compounds of interest in an aqueous solution containing appropriate concentrations of salt (e.g. ammonium sulfate, sodium sulphate). Elution of the desired materials is accomplished by lowering the salt concentration. There are a variety of HIC resins that are commercially available, differing in both backbone and functional chemistries. In general, they can be made to work for plasmid DNA purification, such as, but not limited to, Octyl FF HIC (Amersham Bioscience, Piscataway, N.J.), Phenyl FF HIC (Amersham Bioscience, Piscataway, N.J.), Butyl FF HIC (Amersham Bioscience, Piscataway, N.J.), and Hexyl HIC (Tosoh-Haas, Montgomeryville, Pa.). The HIC step can be a flow through step where the less-hydrophobic molecules flow through the column, i.e., the supercoiled and open circular plasmid flow through the column while RNA, chromosomal DNA, denatured plasmid DNA and endotoxins are retained on the column.

Reversed-phase high-performance liquid chromatography (RP-HPLC) is a technique which can provide rapid analysis and purification of nucleic acid molecules based on their size, chemical properties (charge and hydrophobicity) and conformational constraints, all of which can be exploited via the interactions with reversed-phase solid support. Silica-based reversed-phase chromatography methods perform adequately for separating single-stranded DNA, however, ion-pair RP-HPLC has proved more suited for the analysis and characterization of double-stranded DNA (C. G. Huber and A. Krajete, Anal. Chem. 1999, 71: 3730-3739; A. Apfel et al., Anal. Chem. 1997, 69: 1320-1325).

The analysis of DNA and DNA fragments by ion-pair reversed-phase HPLC can be carried out under non-denaturing, partially denaturing, or fully denaturing conditions. Under non-denaturing conditions, the method provides a means for sequence-independent sizing of DNA fragments of up to 2000 base pairs (C. G. Huber et al., Anal. Chem. 1995, 67: 578-585; K. H. Hecker et al., Biotechniques, 1999, 26: 216-218). Detection of mutations by heteroduplex analysis is possible using partially denaturing conditions (B. Hoogendoom et al., Hum. Genet. 1999, 104: 89-93; M. C. O'Donovan et al., Genomics, 1998, 52: 44-49), while fully denaturing conditions have been shown to allow the study of single stranded DNA fragments of up to 100 nucleotides (P. J. Oefner, J. Chromatogr. 2000, 739: 345-355) and the analysis of RNA (A. Azarani and K. H. Hecker, Nucleic Acids Res. 2001, 29: E7).

Similarly, the use of oligonucleotides for such applications as primers in sequencing techniques, site-specific mutagenesis, hybridization probes as well as for diagnostic and therapeutic purposes (as antisense drugs) has increased the need for analysis and purification methods for these molecules. Oligonucleotides, which typically contain both negatively charged and neutral portions, can be analyzed by ion-pair reversed-phase HPLC under fully denaturing conditions (C. G. Huber et al., Anal. Biochem. 1993, 212: 351-358).

Alkylated solid supports are often used to separate nucleic acids using ion-pair RP HPLC. An alkylated poly(styrenedivinylbenzene) matrix is a support that is commercially available in a cartridge and automated system format (Transgenomic Inc, U.S. Pat. No. 6,488,855. See for examples Azrani A & Hecker K H Nucleic Acids Res 2000; 29(2):1-9; Dickman M J J Chromatogr A 2005; 1076(1-2):83-89; Hecker K H et al J Biochem Biophys methods 2000; 46(1-2):83-93). The alkylated, hydrophobic column matrix interacts directly with the hydrophobic alkyl chains of the triethylammonium acetate (TEAA) contained in the buffer. The positively charged ammonium ions also interact with the negatively charged phosphate backbone of nucleic acids, coating the nucleic acid molecule in a hydrophobic layer. The number of TEAA molecules attached to the nucleic acid molecule is proportional to its length, therefore determining the degree to which the nucleic acid is retained by the hydrophobic solid support. The application of an increasing acetonitrile gradient releases the nucleic acid in order of increasing length, with smaller fragments eluting first. While separation is mostly size-dependent, other factors also can play a role, albeit smaller. For example, exposed bases of single-stranded nucleic acids themselves interact with the column matrix due to their hydrophobic character. Different bases exhibit different degrees of hydrophobicity. This is the basis for the sequence dependence of retention time in IP RP HPLC under certain analysis conditions. Hydrophobicity increases in the order C<G<T<A. Thus, it is expected that single-stranded nucleic acid molecules with a high adenine content exhibit longer retention times than those with high guanine or cytosine content. Polyadenylated mRNA is therefore retained more strongly on the DNASep cartridge than rRNA, which is not polyadenylated (Azrani A & Hecker K H Nucleic Acids Res 2000; 29(2):1-9).

Other non-limiting examples of the use of alkylated supports in nucleic acid separation by ion-pair RP-HPLC include are known to those of skill in the art. Porous silica beads with a mixture of C2 and C18 alkyl ligands resolved DNA fragments ranging from 10 to 3000 pase pairs, using triethylammonium buffers (Eriksson S et al J Chromatogr A 1986; 359:265-274). Double-stranded DNA fragments ranging from 8 to 857 base pairs were efficiently separated on a Poroshell C18 HPLC column (Aligent Technologies) (U.S. Pat. No. 7,125,492), while C4 columns were used to separate mRNAs (van der Mast C A et al. J Chromatogr 1991; 564(1):115-25). Again, separation of the nucleic acid molecules is size-dependent, and based on the relative hydrophobicity of the molecules.

Although separation of nucleic acids using a solid support has historically been performed via column chromatography, methods also have been developed to permit separation using unpacked beads as a solid support. For example, using carboxyl-coated magnetic microparticles, DNA fragments of different sizes can be separated by adjusting the ionic strength or PEG concentration of the elution buffer. Smaller fragments can be eluted from the column with buffers of higher ionic strength or PEG concentration than larger nucleic acid fragments (U.S. Pat. No. 5,898,071).

### E. Nucleic Acid Isolation Using the Modified Solid Supports

According to the methods described herein, nucleic acid molecules can be isolated using solid supports that have been modified through the coupling of a functional ligand to enhance the affinity of the support for the nucleic acid molecules, such as by optimizing the relative hydrophobicity of the solid support to increase binding, while retaining a hydrophilicity that maintains colloidal stability and/or facilitates elution. Any suitable solid support, as described above, can be coupled to an appropriate ligand for modification, although exemplary of these are carboxylated paramagnetic beads. Carboxylated paramagnetic beads suitable for the purposes described herein are commercially available, including, but not limited to, Sera-Mag® beads (Seradyn) and Dynabeads® M-270 Carboxylic Acid (Invitrogen). It is understood that other paramagnetic beads can be carboxylated by methods known to those of skill in the art, and can thus also be made suitable for the purposes described herein. The carboxylated paramagnetic beads are coupled to an amine ligand, such as a hydrophobic amine ligand, more preferably a hydrophobic aliphatic amine ligand. The beads are first washed in a suitable coupling buffer, such as for example, 0.5 M MES or 0.1 M imidazole, or any other suitable buffer known to those of skill in the art. The coupling buffer concentrations can include any that facilitate sufficient hydrolysis of the coupling reagent resulting in formation of an amide bond, but recommended concentrations include 50 mM, 100 mM, 200 mM, 500 mM and 1 M. If only a small quantity of ligand is available for coupling, then the amount of beads should be reduced to maintain the desired bead to ligand ratio. Generally, for microscale applications, a suitable amount, typically about or 2.0 mg, 2.5 mg, 5.0 mg, 7.5 mg, 12.5 mg, 20 mg or 30 mg carboxylated paramagnetic beads are washed prior to coupling, and resuspended in the coupling buffer at approximately 15-25% w/v.

Varying amounts of coupling reagent and ligand are added to the washed beads, and relative ratios of beads, ligand and coupling reagent are modified and optimized to result in the desired degree of coupling. The degree of coupling, for example, can be measured by, and expressed as, the percentage of COO— conversion i.e. the percentage of carboxyl groups that have been converted to form amide bonds. Any other suitable measurement and expression thereof can be used. If a high degree of COO— conversion is desired (for example, to increase the bead hydrophobicity significantly by saturation with hydrophobic ligand), then the coupling reaction should contain a high ligand:bead ratio, whereas a low ligand:bead ratio will generally result in a lower COO— conversion (as desired, for example, to increase the hydrophobicity of the beads only moderately). Any ligand with an amine group can be used, but preferably the ligand is a hydrophobic aliphatic amine. Non-limiting examples of suitable amine ligands include propylamine, HCl propylamine, polypropylamine, butylamine, butoxypropylamine, octylamine, 2-(2-aminoethoxy)ethanol and NH₄(CH₂)₆hexaethylene glycol. The ligand can further have additional moieties attached, such as, for example, a nucleic acid binding motif. Examples of such a ligand, include but not limited to, are NH₄(CH₂)₆-T₆-hexatheylene glycol-MGB, NH₄(CH₂)₆-(hexatheylene glycol)₃-MGB and NH₄(CH₂)₆-T₆-MGB, where T is a thymidine and MGB is a minor groove binder moiety.

The ligand can be dissolved in the coupling buffer at a concentration of, for example, 0.01, 0.05, 0.1, 1.0, 10, 100, 200 or 600 μmol, or any other concentration that will result in the desired degree of coupling. The coupling reagent also is dissolved in the same coupling buffer at concentrations appropriate for that reagent, as known by those of skill in the art. For example, when EDAC HCl is used as the coupling reagent, typical reaction concentrations include, but are not limited to, 0.1, 0.4, 1, 2, 4 or 10 μmol. The dissolved ligand and coupling reagent are added to the washed beads and incubated to allow the reaction to proceed. The reactions are typically incubated with mild agitation, mixing, stirring or rocking, to facilitate adequate mixture of the starting products and any intermediates that accumulate throughout the reaction. Reaction conditions can range from 65° C. for 2 hrs to 4° C. overnight, but for the purposes described herein, typically 65° C. for 2 hrs. The modified beads are then washed thoroughly to remove the unbound ligand, excess coupling agent and any intermediates. Typical wash protocols include, but are not limited to; twice with water at 65° C. and once with water at room temperature; once with coupling buffer at 65° C., twice with water at 65° C. and once with water at room temperature; once with coupling buffer and four times with PBS at room temperature; or once with coupling buffer and four times with Tris pH 7.4 at room temperature. The beads are resuspended at approximately 5% w/v in an appropriate storage buffer, such as for example, 10 mM Tris, pH 7 to 8, or PBS. A biocidial preservative also can be added, such as for example, a ProClin® preservative (available from SigmaAldrich) to a final concentration of 0.05%, or sodium azide to a final concentration of 0.1%.

Prior to the use of the amine-coupled carboxylated paramagnetic beads, or other appropriate solid support, in the isolation of nucleic acids molecules, the beads are typically washed in a buffer, such as for example, 10 mM Tris, pH 7 to 8, or PBS, to remove any preservative used for storage, and again resuspended at 5% w/v. The amount of beads used for the isolation is dictated by the application, increasing as the amount of expected nucleic acid to be isolated increases. Typically, for microscale applications, 2.5 to 15 μl of the coupled beads are used for each nucleic acid sample, although it is understood that this can be scaled up or down to suit the particular application. Nucleic acid can be isolated from a variety of samples, as described previously, including, but not limited to, biological samples, enzymatic reaction samples (such as PCR), purified virus or bacterial cultures. In general, the sample is mixed with the lysis buffer at a 1:2 ratio of sample to buffer. For example, virus in 0.5 ml of standard transport-medium (e.g. M4 transport medium) is mixed with 1 ml of lysis buffer, or 0.5 ml plasma is mixed with 1 ml lysis buffer.

The lysis buffer, as described above, contains a nucleic acid precipitating agent and denaturant in a high ionic-strength solution Generally, the nucleic acid precipitation is effected by a chaotropic agent and an alcohol. A non-limiting example of a suitable lysis buffer is one that contains 2.5 M guanidinium thiocyanate, 50% isopropyl alcohol, 0.5% lauroylsarcosine, 0.05 M Tris HCl, pH 7.0. Other chaotropic agents can be used, including, but not limited to, guanidinium chloride, and sodium chloride, at varying concentrations, although as is well known by those of skill in the art, efficient nucleic acid precipitation does not occur when the concentration is too low. Exemplary concentrations of guanidinium thiocyanate include, for example, 1.5 M, 2 M, 2.5 M, 3 M, 3.5 M and 4 M. At these concentrations, guanidinium thiocyanate, and other chaotropic agents, also function to disrupt cells, viruses, tissues, and associated matrices to release the nucleic acid molecules. It is understood that other reagents in the buffer can be removed, added, or their concentrations changed in a manner that preserves the buffers' ability to efficiently release the nucleic acid from any starting material and precipitate it onto the modified carboxylated beads, or other suitable support. For example, 0.05 M Na citrate, pH 7 can replace 0.05 M Tris, or the concentration of lauroylsarcosine can be increased to 1.5%. Optionally, carrier RNA also is included in the lysis buffer to enhance precipitation of the nucleic acid molecules (Gallagher M L et al Biochem Biophys Res Commun. 1987 Apr. 14; 144(1):271-6.). Typically, 0.5 to 2 μg of carrier polyA RNA is added to 1 ml lysis buffer.

A suitable amount, for example, about 2.5 to 15 μl of the mixture containing 5% w/v modified carboxylated paramagnetic beads in 0.5 ml is added to, for example, about 1.5 ml of the nucleic acid sample. The mixture is mildly agitated, such as for example, by vortexing on a low setting, for about 1, 2, 4, 6 or more minutes. Reducing this incubation time generally reduces the amount of nucleic acid that binds to the support. The lysis buffer is removed from the solid support/nucleic acid complexes. Where the solid support is a paramagnetic bead, this can be achieved by applying a magnetic force to the beads. For example, the beads are collected at the side of a tube by placing a magnet on the external surface of the tube to attract the beads. The lysis buffer can be removed, such as by using a pipette, without disrupting the beads. The magnet is then removed from the vicinity to remove the magnetic force.

The beads are then washed to remove any impurities (e.g. host cell components, proteins, metabolites or cellular debris) that are bound either directly to the bead, or to the adsorbed nucleic acid molecules. The wash buffer solution must have a sufficiently high salt concentration (i.e. be of sufficiently high ionic strength) that the nucleic acid bound to the solid phase carrier is not eluted from the solid phase carrier during the washing process. A suitable salt concentration can be empirically determined and can be as low as about 10 mM, such as 10 mM KCl, and is often greater than about 0.2 M. The components of the wash buffer solution are chosen so that impurities that are bound to the DNA or solid phase carrier are dissolved. The pH and solute composition and concentration of the buffer solution can be varied according to the types of impurities which are expected to be present. For example, a washing protocol useful in the isolation of nucleic acid molecules from viruses includes washing the beads with 1.67 M guanidinium isothiocyanate, 33% isopropyl alcohol, 0.17% lauroylsarcosine, 0.033 M Tris, pH 7.0 once at room temperature, then twice with a solution containing 80% ethanol, 10 mM KCl, 2 mM Tris pH 7.0, 0.2 mM EDTA, pH 8.0. The washing buffer can be optimized for any given application by altering the components or concentrations thereof within limits understood by those of skill in the art.

The bound nucleic acids are eluted from the modified carboxylated paramagnetic beads, or other appropriate solid support, with an elution buffer. Suitable elution buffers are low in ionic strength and include, but are not limited to, nuclease-free water, TE buffer pH 7 to 8, 1 mM KCl with 0.02 mM Na citrate pH 7.0, and 10 mM Tris pH 7 to 8 or 1 mM Tris pH 8, 0.1 mM EDTA. The volume used for elution is such that efficient elution is achieved without diluting the nucleic acid eluate unnecessarily. Too little elution buffer will result in inefficient release of the bound nucleic acid molecules from the support, while too much results in a diluted eluate. Minimally, an equal volume of elution buffer is added to the volume of beads for effective elution (i.e. 10 μl elution buffer is added to 10 μl beads). The nucleic acid molecules can be eluted with 2, 4, 6, 10, 20 or 50 times the volume of elution buffer to beads. For example, 50 μl elution buffer can be used to elute nucleic acid molecules bound to 2.5 to 15 μl beads. Optionally, a multi-step elution can be performed, whereby the beads are treated first with elution buffer to remove the majority of the bound nucleic acid, then again once, twice or more to sequentially remove the remaining bound nucleic acid. It is understood that the majority of the nucleic acid (e.g. 90%) is generally released in the first elution, and that subsequent elution steps yield significant less nucleic acid (e.g. 5%).

The efficiency with which the solid support, for example, modified carboxylated paramagnetic beads, isolates nucleic acid molecules from a given sample is a measurement of two parameters; the purity of the eluted nucleic acid, and the percentage recovery, i.e. what percentage of the input nucleic acid is recovered in the eluate. The purity of the eluted nucleic acid can measured by several means. For example, a sample of the eluate can be electrophoresed on a polyacrylamide gel. The gel can be directly stained with Coomassie Brilliant Blue to visualize any protein impurities, such as for example, contaminating cell wall proteins. Protein contaminants in the eluate also can be detected by measuring the optical density at specific wavelengths. For example, a preparation of DNA is considered to be pure if the ratio of absorbance measured at 260 nm to absorbance measured at 280 nm is 1.8. Protein tends to have a higher absorbance (A) at 280 nm (due mainly to tyrosine and tryptophan) than at 260 nm, whereas nucleic acid has a higher absorbance at 260 nm than at 280 nm. As such, the A260/A280 ratio of proteins is significantly lower than that of DNA, i.e. lower than 1.8. (Felsenfeld G &. Hirschman S, J. Mol. Biol. (1965) 13, 407-427). When looking at the purity of a nucleic acid preparation, an absorbance reading at 230 nm also can be taken. Strong absorbance around 230 nm can indicate that organic compounds or chaotropic salts are present in the purified nucleic acid. nucleic acid. A ratio between the readings at 260 nm and 230 nm (A260/A230) can be used to evaluate the level of salt carryover in the purified nucleic acid. Generally, the lower the ratio the greater the amount of salt that is present. Also, absorption at higher wavelengths (330 nm and higher) is usually caused by light scattering and indicates the presence of particulate matter.

Other assays for specific contaminants also can be employed to determine the purity of the eluted nucleic acid. If nucleic acid is being isolated from a bacterial sample, for example, an assay to detect endotoxin, such as the LAL (Limulus amebocyte lysate) test, can be performed. LAL is an aqueous extract of blood cells (amebocytes) from the horseshoe crab, Limulus polyphemus. LAL reacts with bacterial endotoxin or lipopolysaccharide (LPS), which is a membrane component of Gram negative bacteria. Kits for LAL assays are available commercially, such as for example, the Pyrogent® Plus Gel Clot LAL from Cambrex, and the Limusate® LAL kit from Wako.

The efficiency with which a solid support isolates nucleic acid molecules, with respect to percentage recovery, also can be determined using various methods known to those of skill in the art. The percentage recovery can be expressed as an absolute value, i.e. the absolute percentage of nucleic acid recovered from a starting input sample. In this instance, the amount of nucleic acid present in the starting sample must first be quantified. Alternatively, the percentage recovery can be expressed as a relative percentage i.e relative to the recovery of nucleic acid following isolation using a different solid support. In this instance, quantitiation of the absolute amount of nucleic acid in the starting sample is not required, and only the amount of nucleic acid in the eluates of the samples from each of the solid supports being compared is needed.

The amount of nucleic acid in a given sample can be quantified in a number of ways, and are known to those of skill in the art. For example, the amount of DNA or RNA in a sample can be measured using real-time PCR or real time RT-PCR (reverse transcriptase PCR). The real-time PCR system is based on the detection and quantitation of a fluorescent reporter (Livak K J et al, PCR Methods Appl. 1995 4(6):357-62). This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. A significant increase in fluorescence above the baseline value measured during the 3-15 cycles indicates the detection of accumulated PCR product. A standard curve can be generated using real time PCR of a known amount of control template. This standard curve can then be used to determine the amount of nucleic acid in the experimental samples. Where the amount of RNA in a sample is being detected, the RNA is first reverse transcribed into cDNA before quantitative PCR is performed.

Other methods also can be used to quantitate nucleic acid including, but not limited to, northern blotting (RNA), southern blotting (DNA), capillary electrophoresis, and ribonuclease protection assays (RPA). Another method known to those of skill in the art to quantitate nucleic acids is to label the nucleic acid and then measure the amount of label. For example, the nucleic acid can be labeled with a radioactive substance, such as for example, ³²P or ³H. Non-radioactive labels, including but not limited to digoxigenin (DIG), biotin, and fluorescent dyes, such as for example, fluorescein and rhodamine, and the fluorescent DNA binding dyes Hoechst 33258 and DAPI, also can be used. The labeled nucleic acids are then detected using an appropriate system. For radioactive labels, the level of radioactivity and, by extension, the amount of nucleic acid, can be determined by liquid scintillation (counting, quantitation and detection). Various fluorescence signals can be detected using instruments that provide the appropriate excitation photons, and can detect the resulting emission photons. Quantitation of the fluorescent emission facilitates quantitation of the amount of nucleic acid in the sample.

### F. Combinations and Kits

Combinations and kits containing the combinations optionally including instructions for administration are provided. The combinations include, for example, solid supports suitable for modification according to the methods provided herein and reagents, such as hydrophilic ligands, hydrophobic ligands and coupling reagents such as carbodiimide, for modifying the solid supports. The combinations also can include reagents such as buffers, alcohols such as PEG and chaotropic substances for nucleic acid or other biomolecule isolation.

Additionally provided herein are kits containing the above-described combinations, in which the components are packaged individually or together, and optionally instructions for modifying the solid supports and/or using the solid supports for isolating nucleic acids and optionally other reagents.

The combinations and kits provided herein also can be packaged as articles of manufacture containing packaging material, a combination or kit as provided herein, and a label that indicates that the combination or kit is for modifying solid supports for nucleic acid, protein or other biomolecule isolation or for performing such isolations. The combinations, kits and articles of manufacture provided herein are for use with any of the solid supports and methods and combinations thereof as provided herein.

The kits provided herein can contain packaging materials. Packaging materials for use in such products reagents are well known to those of skill in the art. See, e.g., U.S. Pat. Nos. 5,323,907, 5,052,558 and 5,033,252. Examples of packaging materials include, but are not limited to, blister packs, bottles, tubes, bags, vials, containers, bottles, and any packaging material suitable for the solid supports and associated reagents for modification of the solid supports or for isolation or separation of nucleic acids and other biomolecules using the solid supports as provided herein.

### G. Applications of the Methods

As described in detail above, the beads, other products and methods provided herein can be used to isolate nucleic acids from various samples including, but not limited to, enzymatic reactions, agarose solutions, viral or bacterial material, soil, food, and any body fluid, tissues, cell cultures, cell suspensions and cell lysates. The solid supports and methods described above and exemplified in the Examples below, for example, provide an efficient and cost-effective method to purify nucleic acid, without the use of specialized instruments. Further, the methods described herein can be automated or otherwise adapted, such as for high throughput systems. In one example, reagents and/or samples containing the nucleic acid can be tracked using, for example, barcodes or other similar technology.

Additionally, it will be understood by those of skill in the art that, with minor modification to the described protocols, the hydrophobic carboxylated paramagnetic beads, can be used in other applications, such as for example, those illustrated in the art in reference to reversed phase high performance liquid chromatography (RP-HPLC). RP-HPLC is a chromatographic technique that uses a reversed phase (i.e. hydrophobic) stationary phase to separate compounds dissolved in a polar solution. Numerous RP-HPLC stationary phases have been described, often as columns containing an alkylated solid support, such as octadecylsilane (i.e. C18 chains), octylsilane (i.e. C8 chains), or poly(styrene-divinylbenzene) copolymer beads alkylated with octadecyl groups (PD-DVB-C18), and the applications in which they have been used also can be applied to the modified hydrophobic carboxylated paramagnetic beads described here.

Like RP-HPLC, the beads presented here can be used to purify nucleic acids which, in many applications, is achieved by separating the desired nucleic acid species from unwanted chemicals, salts, proteins, or other undesirable nucleic acids species. For example, following synthesis, oligonucleotide samples contain unwanted salts, chemicals and truncated species. To purify the oligonucleotide, the sample can be loaded into a column containing the support, to which the nucleic acid molecules reversibly bind. The column can then be eluted with gradients of acetonitrile/water/ammonium acetate. The full length oligonucleotides are retained longer on the support, while the salts, chemicals, monophosphates and truncated species are eluted first (See e.g. McFarland et al. (1979) Nucleic Acids Res. 7(4):1067-80; Efimov et al. (1983) Nucleic Acids Res. 11 (23):8369-87; Haupt et al. (1983) J Chromatogr 260:419-427). Other small nucleic acid molecules, such as Speiglemers (a special form of aptamers that are at least partially composed of unnatural L-oligonucleotides), also can be purified and desalted using similar methods (US2005020848; U.S. Pat. No. 5,118,802).

Other species of nucleic acids, such as DNA (Eshaghpour et al. (1978) Nucleic Acids Res 5(1):13-21), RNA (WO2004099411), mRNA (Campbell et al. (1980) Anal Biochem 102(1):153-158; Simonian et al. (1983) J Chromoatogr 266:351-358), tRNA (Drabkin et al. (1978) J Biol Chem 253(17):6233-6241) and RNA and ribozymes (Wincott et al. (1995) Nucleic Acids res 23(14):2677-2684) can be purified using the solid supports, separating the nucleic acids from the unwanted chemicals and molecules in the starting material. Additionally, plasmid DNA can purified from a mixture containing both plasmid DNA and “contaminating” genomic DNA (WO99/29832). Various elution buffers can be employed in these methods, including but not limited to, alkaline buffers containing a linear gradient of potassium chloride in sodium hydroxide, linear gradients of sodium chloride, gradients of acetonitrile, gradients of NaClO₄ and acetonitrile/methanol/water buffers. Peptide nucleic acids (PNAs) also can be purified on the solid supports presented here, using methods adapted from those described for RP-HPLC. For example, following synthesis, PNA-DNA chimeras can be purified on a hydrophobic alkylated support and eluted with TEAA and TEAA/acetonitrile buffers. PNA molecules and PNA-peptide conjugates also can been successfully purified using hydrophobic alkylated solid supports, with elution and separation effected by a linear gradient from 0.1% heptafluorbutyric acid in water to acetonitrile (WO2004029075).

In addition to purification, the alkylated solid supports can be used to separate nucleic acids from one another on the basis of size. This can be achieved by loading the nucleic acid samples onto the solid support (which is contained in a column) in a denaturing alkaline buffer, such KCl/TE/NaOH buffer pH 12.2. After washing, the nucleic acid is eluted with a linear NaCl gradient, and the eluted fractions are monitored by UV absorbance. Such a protocol can successfully separate DNA fragments ranging from 43 base pairs to 1100 base pairs, with the smaller fragments eluting first (Eshaghpour et al. (1978) Nucleic Acids Res 5(1): 13-21). This method can be modified by introducing an ion pair reagent to the buffer to provide positively charged ammonium ions. Most commonly, the ion pair reagent is triethylammonium chloride (TEAA), which associates with the anions from the nucleic acid. The alkyl groups of such ion pair reagents enable the nucleic acids to become hydrophobic and the molecule adsorbs to the stationary phase. The longer the alkyl chain used, the more hydrophobic the nucleic acid and the stronger the interaction with the stationary phase. In addition, larger nucleic acids have a stronger interaction with the stationary phase as the increased length enables an increased number of ion pair molecules to be associated with the nucleic acid. During the separation process, a gradient of acetonitrile is started. As the acetonitrile concentration is increased, the smaller nucleic acids desorb from the solid support first. Finally as the acetonitrile concentration is further increased, the larger nucleic acids are desorbed and travel down the support to the detector. These and similar buffers and protocols can be used, for example, to separate different RNA species in cellular extracts on the basis of size (i.e. tRNA<5S RNA<5.8S RNA<18S RNA<25S RNA; Dickman et al. (2006) RNA 12:691-696), RNA fragments (Azarani et al. (2000) Nucleic Acids Res 29(2):e7), rRNA and mRNA (Azarani et al. (2000) Nucleic Acids Res 29(2):e7; U.S. Pat. No. 6,521,411), and DNA fragments (See e.g. Huber et al. (1993) Nucleic Acids Res 21(5):1061-1066), Huber et al. (1995) Anal Chem 67:578-585, U.S. Pat. No. 6,372,142).

The modified carboxylated solid supports described herein also can be used in more specialized applications, such as described in the art in connection with denaturing HPLC (DHPLC). DHPLC uses the ion pairing reagents and alkylated solid supports, as described above, in addition to increases in temperature, to detect DNA heteroduplices and resulting from mutations and single nucleotide polymorphisms (SNPs). For example, typically two chromosomes as a mixture of PCR products are compared by denaturing the products at 95° C. for 3 minutes, and reannealing over 30 minutes by gradual cooling from 95° C. to 65° C. prior to analysis. In the presence of a mismatch (i.e. due to a mutation in one chromosome), not only the original homoduplices are formed again but, simultaneously, the sense and anti-sense strands of either homoduplex form two heteroduplices. The latter denature more extensively at the analysis temperature of, for example, 56° C. and, therefore, are eluted earlier than the two homoduplices that undergo less pronounced denaturation. Separation of all four species is primarily the result of differences in neighboring stacking interactions (i.e. the interactions of the nucleotide sequences adjacent to the mismatch) that determine the degree of destabilization. Thus, all four double-stranded DNA products can be separated by elution with, for example, TEAA and gradients of acetonitrile (Xiao and Oefner (2001) Hum Mutat 17:439-474). The optimal temperature for mutation detection by these methods also can be determined (Jones et al. (1999) Clin Chem 45(8):1133-1140). This method has been extensively used to detect mutations and variants in genes (See e.g. Gross et al. (2000) Hum Mutat 16:345-353; Giunta et al. (2000) Hum Mutat 16:176-177; Liu et al. (1998) Nucleic Acids Res 26:1396-1400; and Xiao and Oefner (2001) Hum Mutat 17:439-474 for review).

Other applications in which the modified carboxylated solid supports can be employed using methods adapted from those used in DHPLC include, but are not limited to, the sizing of DNA microsatellites for such purposes as human identification and parentage testing (Devaney et al. (2000) Anal Chem 72:858-864), the detection of loss of heterozygosity in tumors (Kleymenova et al. (2000) Mol Carcinog 29:51-58), allelic loss analysis (Gross et al. (2006) Hum Mut [Epub] November 15), the study of self-splicing reactions in ribozymes (Georgopoulos and Leibowitz, (2000) J Chromatogr A 868:109-114), quantitation of gene expression (Hayward_Lester et al. (1999) Genome Res 5:494-499), identification of biallelic polymorphisms on the Y chromosome (Underhill et al. (1997) Genome Res 7:996-1005), gene mapping (Schriml et al. (2000) Biotechniques 28:740-745) and DNA footprinting (US20020137037). Detection of CpG methylation also can be performed using the solid supports described herein. For example, DNA can be extracted from cell lines known to contain either methylated or unmethylated sequences and treated with sodium bisulfite. Sodium bisulfite transforms unmethylated cytidines to uridines, while methylated cytidines are not transformed. Following PCR amplification, the products are analyzed using the alkylated solid supports contained in a column under partially denaturing conditions with heat. Since methylated CpG islands cannot be converted to uridines, PCR products generated from methylated sequences have a higher GC-content and, therefore, melt less than PCR products obtained from unmethylated promoter that contain more AT-base pairs and, therefore, denature more and elute earlier (See e.g. Betz et al. (2004) Hum Mutat 23(6):612-20; Matin et al. (2002) Hum Mutat 20:305-311).

In addition to the analysis of nucleic acids, the modified carboxylated solid supports also can be used in the analysis of proteins and peptides. Such analysis would be based on chromatographic techniques described in the art in connection with RP-HPLC, in which compounds adsorb to the solid supports (generally made up of hydrophobic alkyl chains e.g. C4, C8, and C18) in a high aqueous mobile phase and are eluted from with a high organic mobile phase. The proteins and peptides are separated based on their hydrophobic characteristics by running a linear gradient of the organic solvent. For example, a sample with a mixed protein or peptide population can be loaded onto a column containing the hydrophobic modified carboxylated solid supports in a solution containing 1% formic acid in water. A linear gradient of 0 to 80% acetonitrile in 1% formic acid is then applied to the column over an 80 minute period and the eluted fractions are monitored by UV absorbance. Optionally, the temperature can be varied to, for example, 40° C., 50° C., 60° C., 70° C. or 80° C. to increase resolution, alter selectivity, and decrease the elution time (Dolan et al. (2002) J Chromatogr A 965(1-2):195-205). These methods can be used, for example, for the separation of peptide fragments from enzymatic digests (Chang et al. (1994) J Liq Chromatogr 17:2881-2894), for purification of natural and synthetic peptides (Scarborough et al. (1984) PNAS. 81: 5575-5579), to purify synthetic peptides in milligram and gram quantities (Rivier et al. (1984) J Chromatogr 288:303-328; Lu et al. (2001) BioPharm 14(9):28-35), to separate hemoglobin variants (Schroeder et al. (1985) Hemoglobin 9(4):461-482), to identify grain varieties (Huebner et al. (1990) Cereal Chem. 67(2): 129-135), in the study of enzyme subunits (Robinson et al. (1990) Arch Biochem Biophys 281 (2):239-244) and research cell functions (Sussman (1988) Anal Biochem 169:395-399) and to purify milligram to kilogram quantities of biotechnology-derived polypeptides for therapeutic (U.S. Pat. No. 4,667,016). It will be understood by one of skill in the art that the above-described methods, and any other described in the art in connection with RP-HPLC and its derivative protocols, can be applied using the hydrophobic alkylated and carboxylated paramagnetic beads and other solid supports presented here.

### H. Examples

As described above, the solid supports provided herein are modified to adjust their surface polarity and hydrophobicity in a manner that facilitates recovery and purification of nucleic acid molecules from a sample. The modified solid supports provided herein can facilitate binding of nucleic acid molecules from a sample onto the solid supports, and elution of bound nucleic acid molecules from the solid supports. Exemplified below are methods of modifying solid supports for purifying nucleic acids, modified solid supports for purifying nucleic acids and methods of purifying nucleic acids using the modified solid supports.

The following examples are included for illustrative purposes only and are not intended to limit the scope of the invention(s).

### EXAMPLE 1

### Modification of Carboxylated Paramagnetic Beads Using Hydrophobic Ligands

Paramagnetic beads coated with surface carboxylate groups were modified by coupling various hydrophobic ligands to a fraction of the free carboxylate groups as described below

### Modification of Carboxylated Paramagnetic Beads Using Propylamine or Propylamine Hydrochloride

12.5 mg carboxylated paramagnetic beads (6 μmol carboxylate) (Sera-Mag™ Microparticles, Seradyn, Ind.) were washed two times in 0.1 M imidazole buffer (coupling buffer-EMD Biosciences, Inc. CA), then resuspended in 140 μl 0.1 M imidazole. Additional 0.1 M imidazole was added to achieve a final volume of 312 μl after the addition of all reactants. To the suspension was further added propylamine or propylamine hydrochloride (hydrophobic ligand—Sigma-Adrich Inc., MO), followed by the addition of coupling reagent, 1-ethyl-3-(3′-dimethylaminopropyl) carbodiimide (EDAC) (EMD Biosciences inc., CA). Thus, the ligands and coupling reagent were added and additional buffer was added to achieve a final volume of 312 μl. The relative amounts of beads (measured as the amount of free surface carboxylate, see Table 1 below), hydrophobic ligand and EDAC (coupling parameters) were varied depending on the fraction of carboxyl residues to be modified with the ligand. Exemplary coupling parameters are set forth in Table 1.

TABLE 1
Coupling Parameters for modification of carboxyl residues
on the surface of Sera-Mag ™ beads with
propylamine or propylamine hydrochloride

| | | Coupling parameters | | |
|---|---|---|---|---|
| | | | EDAC | |
| Max % of COO− | | Carboxylate | HCl | Ligand |
| converted | Ligand | (μmol) | (μmol) | (μmol) |
| 0.3 | propylamine | 6 | 0.4 | 0.02 |
| 1.7 | propylamine | 6 | 0.4 | 0.1 |
| 6.7 | propylamine | 6 | 0.4 | 1-4 |
| 66.7 | propylamine | 6 | 4 | 4 |
| 100 | propylamine | 6 | 10 | 12 |
| 1.7 | HCl propylamine | 6 | 0.4 | 0.1 |
| 6.7 | HCl propylamine | 6 | 0.4 | 1 |
| 16.7 | HCl propylamine | 6 | 1 | 1 |
| 67 | HCl propylamine | 6 | 4 | 1 |
| 100 | HCl propylamine | 6 | 10 | 10 |

The reactions were incubated at 65° C. for 4 hours with mixing. The beads were then washed once with 0.1 M imidazole at 65° C., two times with water at 65° C., and once with water at room temperature (22° C.). The resulting modified beads were resuspended at 5% w/v in 10 mM Tris, pH 7.0, with 0.05% ProClin™ as a preservative. The beads were stored at 4° C. until use.

### EXAMPLE 2

### Modification of Carboxylated Paramagnetic Beads Using Octylamine

12.5 mg carboxylated paramagnetic beads (6 μmol carboxylate) (Sera-Mag™ Microparticles, Seradyn, Ind.) were washed two times in 0.1 M imidazole buffer (coupling buffer-EMD Biosciences inc., CA), then resuspended in 140 μl 0.1 M imidazole. Additional 0.1 M imidazole was added to the suspension, to a final volume of 312 μl. To the suspension was further added octylamine (hydrophobic ligand-Sigma-Aldrich Inc., MO), followed by the addition of coupling reagent, 1-ethyl-3-(3′-dimethylaminopropyl) carbodiimide (EDAC) (EMD Biosciences inc., CA). The relative amounts of bead (measured as the amount of free surface carboxylate, see Table 2 below), hydrophobic ligand and EDAC (coupling parameters) were varied depending on the fraction of carboxyl residues to be modified with the ligand. Exemplary coupling parameters are set forth in Table 2 below:

TABLE 2
Coupling Parameters for modification of carboxyl residues on
the surface of Sera-Mag ™ beads with octylamine

| | | Coupling parameters | | |
|---|---|---|---|---|
| | | | EDAC | |
| Max % of COO− | | Carboxylate | HCl | Ligand |
| converted | Ligand | (μmol) | (μmol) | (μmol) |
| 6.7 | octylamine | 6 | 0.4 | 600 |
| 33 | octylamine | 6 | 2 | 600 |
| 100 | octylamine | 6 | 10 | 600 |

The reactions were incubated at 65° C. for 4 hours with mixing. The beads were then washed once with 0.1 M imidazole at 65° C. and two times with water at 65° C. The resulting modified beads were resuspended at 5% w/v in 10 mM Tris, pH 7.0, with 0.05% ProClin™ as a preservative. The beads were stored at 4° C. until use.

### EXAMPLE 3

### Modification of Carboxylated Paramagnetic Beads Using Aliphatic Hydrophobic Ligands Containing Minor Groove Binders

2.5 mg carboxylated paramagnetic beads (1.2 μmol carboxylate) (Sera-Mag™ Microparticles, Seradyn, Ind.) were washed two times in 0.1 M 2-(N-morpholino) ethane sulfonic acid monohydrate (MES) buffer (coupling buffer; Research Organics, Inc.). The buffer was removed and the beads were resuspended to a final volume of 62.5 ul in 0.1 M MES containing 1-ethyl-3-(3′-dimethylaminopropyl) carbodiimide (EDAC) (EMD Biosciences inc., CA) and one of the ligands listed below
a. NH₂(CH₂)₆—O—P(═O)(O—)—O—(CH₂ CH₂O)₆—P(═O)(O⁻)—O-MGB
b. NH₂(CH₂)₆—O—P(═O)(O⁻)—O-{(CH₂ CH₂O)₆}₃—P(═O)(O⁻)—O-MGB; or
c. NH₂(CH₂)₆—O—P(═O)(O—)—O-TTTTTT-O—P(═O)(O⁻)—O-MGB

The minor groove binders (MGB) were selected from among the following:

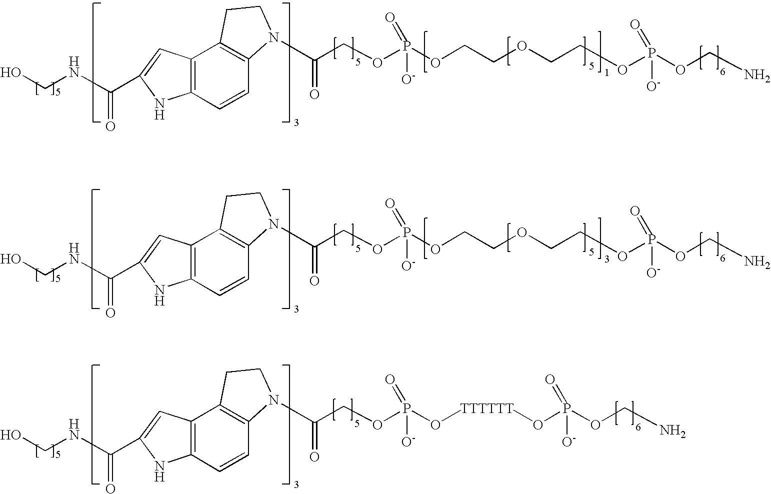

The relative amounts of beads (measured as the amount of free surface carboxylate, see Table 3 below), hydrophobic ligand and EDAC (coupling parameters) were varied depending on the fraction of carboxyl residues to be modified with the ligand. Exemplary coupling parameters are set forth in Table 3 below:

TABLE 3
Coupling Parameters for modification of carboxyl residues on
the surface of Sera-Mag ™ beads with hydrophobic
ligands containing minor groove binders (MGB)

| | | Coupling parameters | | |
|---|---|---|---|---|
| Max % of | | | EDAC | |
| COO− | | Carboxylate | HCl | Ligand |
| converted | Ligand | (μmol) | (μmol) | (μmol) |
| 0.5 | NH₂—(CH₂)₆—O—P(═O)(O⁻)—(C18)-MGB | 2.4 | 4.8 | 0.012 |
| 1 | NH₂—(CH₂)₆—O—P(═O)(O⁻)—(C18)-MGB | 2.4 | 4.8 | 0.024 |
| 1 | NH₂—(CH₂)₆—O—P(═O)(O⁻)—(C18)-MGB | 1.2 | 2.4 | 0.012 |
| 2 | NH₂—(CH₂)₆—O—P(═O)(O⁻)—(C18)-MGB | 1.2 | 2.4 | 0.024 |
| 4 | NH₂—(CH₂)₆—O—P(═O)(O⁻)—(C18)-MGB | 1.2 | 2.4 | 0.05 |
| 5 | NH₂—(CH₂)₆—O—P(═O)(O⁻)—(C18)-MGB | 1.2 | 2.4 | 0.06 |
| 20 | NH₂—(CH₂)₆—O—P(═O)(O⁻)—(C18)-MGB | 1.2 | 2.4 | 0.24 |
| 5 | NH₂—(CH₂)₆—O—P(═O)(O⁻)—T6-MGB | 1.2 | 2.4 | 0.06 |
| 20 | NH₂—(CH₂)₆—O—P(═O)(O⁻)—T6-MGB | 1.2 | 2.4 | 0.24 |

C18 is O—(CH₂CH₂O)₆—P(═O)(O⁻)—O—and T6 is TTTTTT—O—P(═O)(O⁻)—O—

The reactions were incubated at 65° C. for 4 hours with mixing. The beads were then washed once with 0.1 M imidazole at 65° C. and two times with water heated to 65° C. The resulting modified beads were resuspended at 5% w/v in 10 mM Tris, pH 7.0, with 0.05% ProClin™ as a preservative. The beads were stored at 4° C. until use.

### EXAMPLE 4

### Modification of Carboxylated Paramagnetic Beads Using Aliphatic Amines Coupled to Thymidine

20 mg carboxylated paramagnetic beads (10 μmol carboxylate) (Sera-Mag™ Microparticles, Seradyn, Ind.) were washed two times in 0.1 M 2-(N-morpholino) ethane sulfonic acid monohydrate (MES) buffer (coupling buffer; Research Organics, Inc.). The buffer was removed and the beads were resuspended to a final volume of 500 ul 0.1 M MES containing 1-ethyl-3-(3′-dimethylaminopropyl) carbodiimide (EDAC) (EMD Biosciences inc., CA) and one of the following hydrophobic ligands:

a. NH₂(CH₂)₆—O—P—(═O)(O⁻)—O—(CH₂ CH₂O)₆ O—P—(═O)(O—)—O-T-OH (TriLink BioTechnologies, Inc., CA); or
b. NH₂(CH₂)₆—O—P—(═O)(O—)—O—(CH₂ CH₂O)₆—P—(═O)(O⁻)—O-T₆-OH (TriLink BioTechnologies, Inc., CA). The relative amounts of beads (measured as the amount of free surface carboxylate, see Table 4 below), hydrophobic ligand and EDAC (coupling parameters) were varied depending on the fraction of carboxyl residues to be modified with the ligand. Exemplary coupling parameters are set forth in Table 4 below:

TABLE 4
Coupling Parameters for modification of carboxyl residues on the surface
of Sera-Mag ™ beads with aliphatic amines coupled to thymidine

| | | Coupling parameters | | |
|---|---|---|---|---|
| Max % of | | | EDAC | |
| COO− | | Carboxylate | HCl | Ligand |
| converted | Ligand | (μmol) | (μmol) | (μmol) |
| 1 | NH₂—(CH₂)₆—O—L—(CH₂CH₂O)_{6—}L—T6—OH | 2.4 | 4.8 | 0.024 |
| 2 | NH₂—(CH₂)₆—O—L—(CH₂CH₂O)_{6—}L—T6—OH | 2.4 | 4.8 | 0.048 |
| 1 | NH₂—(CH₂)₆—O—L—(CH₂CH₂O)_{6—}L—T—OH | 2.4 | 4.8 | 0.024 |
| 2 | NH₂—(CH₂)₆—O—L—(CH₂CH₂O)_{6—}L—T—OH | 2.4 | 4.8 | 0.048 |

L is —P—(═O)(O⁻)—O

The reactions were incubated at 65° C. for 4 hours with mixing. The beads were then washed once with 0.1 M imidazole at 65° C. and two times with water heated to 65° C. The resulting modified beads were resuspended at 5% w/v in 10 mM Tris, pH 7.0, with 0.05% ProClin™ as a preservative. The beads were stored at 4° C. until use.

II. Nucleic Acid Isolation Using Paramagnetic Beads Modified with Hydrophobic Ligands

The modified carboxylated paramagnetic beads prepared as described in Section I above were tested for their ability to capture and recover nucleic acids from liquid samples by monitoring nucleic acid binding and elution from the beads (see Example 5).

### EXAMPLE 5

### Protocols for Capturing and Recovering Nucleic Acids Using Carboxylated Beads Modified with Hydrophobic Ligands

a. Purified DNA and RNA

E. coli DNA was purified by standard methods from cultured cells using the PUREGENE DNA Isolation Kit (GentraSystems, MN). RNA was obtained from Prodesse (Prodesse, Wisconsin)

b. Radiolabeled DNA and RNA

E. coli DNA purified by standard methods was radiolabeled using ³²P dCTP. A whole genome amplification procedure was used to incorporate the radiolabeled nucleotide. 10-20 pg of E. coli DNA was heat denatured and then incubated with non-radioactive dNTPs (200 uM each dGTP, dTTP, dATP, 23 uM dCTP) (Amersham, now GE Bioscience), ³²P dCTP (32 uM, specific activity ˜400 Ci/mmol)(Amersham), random hexamers (˜60 uM) (IDT, IO), 1× phi29 polymerase buffer (New England Biolabs, MA), and 5 units of phi29 polymerase (New England Biolabs, Mass.) for 17-22 hours at 30° C. The reaction mix was heated for 5 min at 65° C., and the radiolabeled DNA was purified on spin columns (P30, BioRad, CA).

The amount of radiolabel incorporated into DNA was determined by spotting dilutions of the reaction mix, both pre- and post-spin column, onto a Zetaprobe nylon membrane (BioRad, CA). The membranes were washed 3 times with a phosphate buffer to remove unincorporated ³²P-dCTP. The washed and dried membranes were placed into scintillation fluid (Econosafe Scintillation Fluid, Research Products International Corp., IL) and counted in a scintillation counter (1600TR Liquid Scintillation Analyzer, Packard BioScience, IL). Typically after the spin column purification, 95-99% of the radioactivity remained bound to the membrane. Aliquots analysed by gel electrophoresis and autoradioagraphy indicated that the majority of the labelled DNA was greater than 4 kb in length. Aliquots of this radiolabeled DNA were used to study the binding to and elution from the carboxylated beads modified with hydrophobic ligands. Typically 100,000 to 200,000 cpm were used for a single reaction.

Radiolabeled RNA was prepared by in vitro transcription. cDNA encoding a portion of the human parainfluenza virus 2 (HPIV-2) hemagglutinin-neuraminidase gene was cloned into pCRII-TOPO (Invitrogen, CA). The plasmid was linearized and used as a template for T7 RNA polymerase (Maxiscript Transcription Kit, Ambion, Tex.) in the presence of ³²P-CTP and non-radioactive nucleotides as recommended by the kit manufacturer. The reaction was incubated for 20 min at 37° C. and was stopped by the addition of EDTA to 0.5 mM. The radiolabeled RNA was purified using spin columns (NucAway Spin Column, Ambion, Tex.). As described above, the amount of radiolabeled RNA was determined by spotting onto a Zetaprobe membrane (Biorad, CA), washing away the unbound nucleotide and then counting the membrane bound radiolabeled RNA with scintillation fluid (Econosafe Scintillation Fluid, Research Products International Corp., IL) in a scintillation counter (1600TR Liquid Scintillation Analyzer, Packard BioScience, IL). Typically 100000 to 200000 cpm were used in each binding reaction. Purified RNA or DNA Note; the amount varied depending on the experiment. With pure RNA we often used 50,000 copies. With the armored RNA, which is hard to quantitate we used a higher copy number. With unlabeled DNA, the amount was ˜10⁶ genome equivalents/rx) was added to 1 ml lysis buffer containing 2.5 M guanidinium thiocyanate, 50% isopropanol, 0.25% lauroyl sarcosine, 0.05 M Na citrate, pH 7.0, and 2 μg/ml polyA RNA. To the DNA or RNA solution was then added a 0.5 ml aliquot of M4 transport medium (Remel, Lenexa, Kans.) and 2.4 μl of a 5% w/v solution of beads (unmodified neutral beads

(MagMAX™ magnetic beads, Ambion, Inc., TX), unmodified carboxylated beads (Sera-Mag™ Microparticles, Seradyn, Ind.) or carboxylated beads modified with a hydrophobic ligand prepared as described in Examples 1-4 above). The mixture was vortexed gently for 4 minutes and the magnetic beads were collected at the side of the tube with a magnet. The lysis buffer was removed and the beads were washed two times with a wash buffer containing 1.67 M guanidinium isothiocyanate, 33% isopropanol, 0.17% lauroyl sarcosine and 0.033 M Na citrate, pH 7.0. The beads were washed a further two times with a wash buffer containing 80% ethanol, 10 mM KCl, 2 mM Tris pH 7.0 and 0.2 mM EDTA, pH 8.0. The second wash buffer was removed and the beads were treated with 50 μl of an elution buffer containing 1 mM KCl and 0.2 mM Na citrate, pH 7.0.

Heating was independent of the particular bead type. Recovery of nucleic acids (particularly DNA) was improved if heat (65° C.) during the elution step.) In some experiments, the beads were heated to 65° C. in the elution buffer The binding and elution properties of the unmodified or modified carboxylated paramagnetic beads were monitored by measuring the recovery of the ³²P-radiolabeled nucleic acids using scintillation counting. When non-radiolabeled nucleic acids were used, aliquots of the eluted nucleic acids were added to a real time, one step reverse transcriptase-polymerase chain reaction (RT-PCR) to determine recovery of RNA or to a real time polymerase chain reaction (PCR) to determine the recovery of DNA.

C. Encapsulated RNA

RNA encapsulated in MS2 phage head proteins (Xenopus Armored RNA Quant or HPIV-1 HA armored RNA, Ambion Diagnostics, Inc., TX)) was used as surrogate virus. Approximately 10000 copies of the armored RNA was added to 0.5 ml M4 transport medium (Remel, Lenexa, Kans.). One ml lysis buffer containing 2.5 M guanidinium thiocyanate, 50% isopropanol, 0.25% lauroyl sarcosine, 0.05 M Na citrate, pH 7.0, 2 μg/ml polyA RNA and 2.4 μl of a 5% solution of modified or unmodified beads (as described in Example 5a) were then added to the RNA-containing solution. The mixture was vortexed gently for 4 minutes and the magnetic beads were collected at the side of the tube with a magnet. The lysis buffer was removed and the beads were washed two times with a wash buffer containing 1.67 M guanidinium isothiocyanate, 33% isopropanol, 0.17% lauroyl sarcosine and 0.033 M Na citrate, pH 7.0. The beads were washed a further two times with a wash buffer containing 80% ethanol, 10 mM KCl, 2 mM Tris pH 7.0 and 0.2 mM EDTA, pH 8.0.

The second wash buffer was removed and the beads were treated with 50 μl of an elution buffer containing 1 mM KCl and 0.2 mM Na citrate, pH 7.0. Aliquots of the eluted nucleic acids were added to a real time, one step reverse transcriptase-polymerase chain reaction (RT-PCR) to determine recovery of RNA or to a real time polymerase chain reaction (PCR) to determine the recovery of DNA.

d. Complex Samples

The ability of modified carboxylated paramagnetic beads to capture and purify DNA and RNA from nasopharyngeal specimens was examined. One half ml aliquots of nasopharyngeal specimens were briefly pretreated with detergent and protease and then added to 1 ml lysis buffer containing 2.5 M guanidinium thiocyanate, 50% isopropanol, 0.75% or 1% lauroyl sarcosine, 0.05 M Tris HCl, pH 7.0, 1 μg/ml polyA RNA, and 2.4 to 15 μl of a 5% solution of modified or unmodified beads. Radiolabeled DNA was added (as described in Example 5b). The mixture was vortexed gently for 4 minutes and the magnetic beads were collected at the side of the tube with a magnet. The lysis buffer was removed and the beads were washed two times with a wash buffer containing 1.67 M guanidinium isothiocyanate, 33% isopropanol, 0.17% lauroyl sarcosine and 0.033 M Na citrate, pH 7.0. The beads were washed a further two times with a wash buffer containing 70% ethanol, 10 mM KCl, 2 mM Tris pH 7.0 and 0.2 mM EDTA, pH 8.0 The second wash buffer was removed and the beads were treated with 50 μl of an elution buffer containing 1 mM Tris, pH 8.0 and 0.1 mM EDTA. The binding and elution properties of the modified carboxylated paramagnetic beads were monitored by determining the recovery of ³²P-radiolabeled DNA using scintillation counting. The recovery of the radiolabeled DNA from the modified carboxylated beads was compared to the amount of DNA recovered from unmodified carboxylated paramagnetic beads.

### EXAMPLE 6

### Nucleic Acid Recovery from Modified Beads

a. Recovery from Charged (Carboxylated) Unmodified Beads vs. Neutral Unmodified Beads

The relative recovery of nucleic acids using charged magnetic beads vs. neutral magnetic beads was compared by measuring the capture and elution of a human parainfluenza virus (HPIV) RNA or Xenopus armored RNA using a neutral unmodified magnetic bead (MagMAX™) and a charged (carboxylated) unmodified magnetic bead (Sera-Mag™). HPIV RNA or Xenopus armored RNA was isolated using either the MagMAX™ beads or the Sera-Mag™ beads and their recovery monitored as described in Example 5b.

Recovery of HPIV RNA using the charged beads was about 123% (Table 5 below, Jan. 25, 2005) relative to recovery from the neutral beads. While the percent recovery of HPIV RNA from the sample using neutral beads was about 56% on average, the amount of RNA recovered using the charged beads was an average of about 69%.

TABLE 5
Recovery of human parainfluenza virus 1 (HPIV-1) RNA during
sample preparation using a neutral bead (MagMAX ™,
Ambion) and a carboxylated bead (Sera-Mag ™, Seradyn).

| | | Avg # of | | |
|---|---|---|---|---|
| | Avg # of | copies in | Calculated | Recovery relative |
| | copies | positive | recovery | to neutral beads |
| Bead Type | detected | control | (%) | (%) |
| MagMAX ™ | 356 | 638 | 56 | |
| Sera-Mag ™ | 455 | 658 | 69 | 123 |

Recovery of Xenopus armored RNA using the charged beads was about 133% (Table 6 below) relative to recovery from the neutral beads. The amount of RNA recovered from the sample using neutral beads was about 50-54% of the total amount present in the sample, while the amount recovered using the charged beads was about 61-78% of the total amount present.

TABLE 6
Recovery of RNA from an Armored RNA (protected) construct

| | | Recovery relative |
|---|---|---|
| | % | to neutral beads |
| Bead Type | recovery | (%) |
| MagMAX ™ | 54% | |
| Sera-Mag ™ | 78% | 144 |
| MagMAX ™ | 50% | |
| Sera-Mag ™ | 61% | 122 |

b. Recovery from Charged (Carboxylated) Unmodified Beads vs. Charged (Carboxylated) Beads Modified with Hydrophobic Ligands

The charged carboxylated unmodified beads described in Example 6a (Sera-Mag™) were modified using hydrophobic ligands as described in Examples 1-4. Nucleic acid recovery using the modified beads was compared to the recovery under comparable conditions using the charged unmodified beads. As noted below, for each hydrophobic ligand, an optimum level (neither too low nor too high) of modification of the carboxylate residues was identified for maximum recovery of DNA or RNA.

i. Recovery from Solutions Spiked with Naked DNA or RNA

Carboxylated paramagnetic beads modified by coupling to (1) propylamine; (2) propylamine hydrochloride; (3) octylamine; (4) aliphatic hydrophobic chains conjugated to minor groove binders (MGBs); or (5) aliphatic hydrophobic chains conjugated to thymidine(s) as described in Examples 1-4 were used to capture, elute and recover naked DNA or RNA spiked into solutions as described in Example 5a. Nucleic acid recovery was monitored as described in Example 5a., and the results are as follows:

(1) Propylamine: Sera-Mag™ beads modified by coupling to propylamine resulted in RNA recovery of 95-100% relative to the amount recovered using unmodified Sera-Mag™ beads. The amount of DNA recovered using the propylamine modified beads varied from about 67% to about 107% relative to the amount recovered using unmodified beads.

TABLE 7
Recovery of radiolabeled DNA and RNA using propylamine
coupled to Sera-Mag ™ beads

| | | | | | | % |
|---|---|---|---|---|---|---|
| | | | | | % re- | recovered |
| | | | | | covered | relative |
| Nucleic | Max % | % | % | % | Sera- | to Sera- |
| acid type | coupled | bound | eluted | recovered | Mag | Mag |
| 32P-DNA | 0.3 | 97 | 41 | 39 | 46 | 85 |
| 32P-DNA | 6.7 | 93 | 46 | 43 | 41 | 105 |
| 32P-DNA | 6.7 | 94 | 47 | 44 | 41 | 107 |
| 32P-DNA | 6.7 | 97 | 32 | 46 | 51 | 90 |
| 32P-DNA | 6.7 | 97 | 32 | 31 | 46 | 67 |
| 32P-RNA | 1.7 | 96 | 75 | 73 | 76 | 96 |
| 32P-RNA | 1.7 | 96 | 79 | 76 | 76 | 100 |
| 32P-RNA | 6.7 | 96 | 75 | 72 | 76 | 95 |
| 32P-RNA | 6.7 | 96 | 79 | 76 | 76 | 100 |

(2) Propylamine Hydrochloride: Sera-Mag™ beads modified by coupling to propylamine hydrochloride resulted in RNA recovery of 99% (at 16.7% modification of carboxylate residues) to 113% (at 1.7% modified carboxylate residues), relative to the unmodified beads. The amount of DNA that was recovered using the propylamine hydrochloride-modified beads varied from about 89% at 1.7% modified carboxylate to a maximum of 92% at 6.7% modified carboxylate, down again to about 89% when 16.7% of the carboxylate residues were modified, relative to the recovery using unmodified beads.

TABLE 8
Recovery of radiolabeled DNA and RNA using propylamine
hydrochloride coupled to Sera-Mag ™ beads

| | | | | | | % |
|---|---|---|---|---|---|---|
| | | | | | % re- | recovered |
| | | | | | covered | relative |
| Nucleic | Max % | % | % | % | Sera- | to Sera- |
| acid type | coupled | bound | eluted | recovered | Mag | Mag |
| 32P-DNA | 1.7 | 97 | 48 | 47 | 53 | 89 |
| 32P-DNA | 1.7 | 97 | 49 | 48 | 53 | 91 |
| 32P-DNA | 6.7 | 97 | 48 | 47 | 53 | 89 |
| 32P-DNA | 6.7 | 98 | 48 | 47 | 53 | 89 |
| 32P-DNA | 6.7 | 98 | 49 | 48 | 53 | 91 |
| 32P-DNA | 6.7 | 98 | 50 | 49 | 53 | 92 |
| 32P-DNA | 16.7 | 97 | 48 | 47 | 53 | 89 |
| 32P-DNA | 16.7 | 97 | 48 | 47 | 53 | 89 |
| 32P-RNA | 1.7 | 97 | 89 | 86 | 76 | 113 |
| 32P-RNA | 1.7 | 96 | 81 | 79 | 76 | 104 |
| 32P-RNA | 6.7 | 96 | 80 | 76 | 76 | 100 |
| 32P-RNA | 6.7 | 96 | 79 | 76 | 76 | 99 |
| 32P-RNA | 6.7 | 97 | 81 | 78 | 76 | 103 |
| 32P-RNA | 6.7 | 96 | 81 | 78 | 76 | 99 |
| 32P-RNA | 16.7 | 97 | 80 | 77 | 76 | 101 |
| 32P-RNA | 16.7 | 97 | 82 | 79 | 76 | 99 |

(3) Aliphatic hydrophobic chains conjugated to minor groove binders (MGBs): Sera-Mag™ beads modified by coupling to either: NH₂(CH₂)₆—O—P(═O)(O⁻)—O—(CH₂ CH₂O)₆—P(═O)(O⁻)—O-MGB (A) NH₂(CH₂)₆—O—P(═O)(O⁻)—O-TTTTTT-O—P(═O)(O⁻)—O-MGB (B)

prepared as described in Example 3, were used to capture, elute and recover DNA according to the protocol in 1 a. DNA recovery using beads modified with ligand A varied from about 88% at 5% modified carboxylate, to a maximum of about 180% at 1% modified carboxylate, to about 119% at 0.5% modified carboxylate, relative to DNA recovery using unmodified beads. DNA recovery using beads modified with ligand B varied from about 109% at 5% modified carboxylate, to a maximum of about 186% at 20% modified carboxylate, relative to DNA recovery using unmodified beads. The results are set forth below in Table 9.

TABLE 9
Recovery of ³²P labeled DNA from beads modified with aliphatic
hydrophobic chains conjugated to minor groove binders (MGBs):

| | | | | | | % recovered |
|---|---|---|---|---|---|---|
| | Max % | % | % | | % recovered | relative to |
| Ligand | coupled | bound | eluted | % recovered | Sera-Mag | Sera-Mag |
| NH₂(CH₂)₆—PO₃⁻—(CH₂CH₂O)₆—PO₃⁻- | 0.5 | 91 | 55 | 50 | 42 | 119 |
| MGB | | | | | | |
| NH₂(CH₂)₆—PO₃⁻—(CH₂CH₂O)₆—PO₃⁻- | 1 | 87 | 73 | 63 | 35 | 180 |
| MGB | | | | | | |
| NH₂(CH₂)₆—PO₃⁻—(CH₂CH₂O)₆—PO₃⁻- | 1 | 87 | 59 | 52 | 39 | 133 |
| MGB | | | | | | |
| NH₂(CH₂)₆—PO₃⁻—(CH₂CH₂O)₆—PO₃⁻- | 1 | 88 | 59 | 51 | 39 | 131 |
| MGB | | | | | | |
| NH₂(CH₂)₆—PO₃⁻—(CH₂CH₂O)₆—PO₃⁻- | 2 | 62 | 76 | 47 | 50 | 94 |
| MGB | | | | | | |
| NH₂(CH₂)₆—PO₃⁻—(CH₂CH₂O)₆—PO₃⁻- | 2 | 57 | 80 | 45 | 35 | 129 |
| MGB | | | | | | |
| NH₂(CH₂)₆—PO₃⁻—(CH₂CH₂O)₆—PO₃⁻- | 2 | 61 | 76 | 46 | 35 | 131 |
| MGB | | | | | | |
| NH₂(CH₂)₆—PO₃⁻—(CH₂CH₂O)₆—PO₃⁻- | 4 | 43 | 82 | 35 | 35 | 100 |
| MGB | | | | | | |
| NH₂(CH₂)₆—PO₃⁻—(CH₂CH₂O)₆—PO₃⁻- | 5 | 53 | 83 | 44 | 50 | 88 |
| MGB | | | | | | |
| NH2—(CH2)6—PO3⁻-T6-MGB | 5 | 50 | 77 | 38 | 35 | 109 |
| NH2—(CH2)6—PO3⁻-T6-MGB | 20 | 85 | 76 | 65 | 35 | 186 |
| NH2—(CH2)6—PO3⁻-T6-MGB | 20 | 79 | 73 | 58 | 35 | 166 |

ii. Recovery from Encapsulated RNA

Carboxylated paramagnetic beads (Sera-Mag™) were coupled with propylamine, propylamine hydrochloride or octylamine as described in Examples 1 and 2. HPIV RNA transcripts from artificial viral particles were purified using these beads as described in Example 5b, and aliquots of the eluted RNA were reverse transcribed and quantitated with real time PCR to determine the recovery relative to that achieved using unmodified beads.

(1) Propylamine: The maximum percentage of bead surface carboxylate residues that were modified using propylamine ranged from 0.3% to 100%. The average nucleic acid recovery using the resulting propylamine-modified beads was 104%. The most effective isolation was achieved with propylamine-modified beads that had a maximum carboxylate modification of 0.3%, which resulted in 119% RNA recovery relative to that observed using unmodified beads. The lowest RNA recovery, 88% relative to that obtained using unmodified beads, was observed using propylamine-modified beads that had a maximum carboxylate modification level of 100%.

The ability of propylamine-coupled carboxylated paramagnetic beads to purify RNA from phage head proteins also was determined using the methods described above. Aliquots of the eluted RNA were reverse transcribed and quantitated with real time PCR, and copy numbers were determined based on a standard curve that was produced using real time PCR on known quantities of purified RNA. Propylamine-modified beads bound and recovered approximately 500 more RNA transcripts than the non-modified beads, amounting to an increase in RNA recovery of 15-25%.

TABLE 10
Recovery of Encapsulated DNA from beads modified using propylamine

| | | | | Recovery - |
|---|---|---|---|---|
| | | Amount recovered | Amount recovered | modified beads |
| | Max % | from modified | from unmodified | relative to |
| | COO⁻ | Sera-Mag ™ (avg # | Sera-Mag (avg # | unmodified beads |
| Ligand | converted | of copies) | of copies) | (%) |
| propylamine | 0.3 | 3652 | 3067 | 119 |
| propylamine | 1.7 | 2244 | 2309 | 97 |
| propylamine | 6.7 | 2836 | 2644 | 107 |
| propylamine | 16.7 | 2434 | 1906 | 128 |
| propylamine | 100 | 2812 | 2347 | 120 |

(2) Propylamine hydrochloride: The maximum percentage of bead surface carboxylate residues that were modified using propylamine hydrochloride ranged from 7% to 100%. An average nucleic acid recovery of 119%, relative to that recovered using unmodified beads, was achieved. The most effective isolation was achieved with propylamine hydrochloride-modified beads that had a maximum carboxylate modification of 67%, which resulted 28% RNA recovery relative to that observed using unmodified beads. HPIV RNA isolation using modified beads with 7% maximum carboxylate modification and 100% maximum carboxylate modification resulted in 109% and 120% recovery respectively, relative to the RNA recovery obtained using unmodified beads.

TABLE 11
Recovery of Encapsulated DNA from beads modified using HCl-
propylamine

| | | Amount | Amount | Recovery - |
|---|---|---|---|---|
| | | recovered from | recovered from | modified beads |
| | | modified Sera- | unmodified Sera- | relative to |
| | Max % COO⁻ | Mag ™ (avg # of | Mag (avg # of | unmodified |
| Ligand | converted | copies) | copies) | beads (%) |
| HCl-propylamine | 6.7 | 3104 | 3386 | 92 |
| HCl-propylamine | 16.7 | 2434 | 1906 | 128 |
| HCl-propylamine | 100.0 | 2812 | 2347 | 120 |

(3) Octylamine: The maximum percentage of bead surface residues that were modified using octylamine ranged from 7% to 100%. The most effective isolation was achieved with octylamine-modified beads that had a maximum carboxylate modification of 7%, which resulted in an average of 112% HPIV RNA recovery, relative to that observed using unmodified beads. The RNA recovery at 33% maximum carboxylate residue modification decreased to an average value that was comparable to that obtained using unmodified beads, and the RNA recovery value decreased even further, to 1% relative to unmodified beads, when 100% of the carboxylate residues were modified with octylamine.

The ability of octylamine-coupled carboxylated paramagnetic beads to purify RNA from phage head proteins also was determined using the methods described above. Aliquots of the eluted RNA were reverse transcribed and quantitated with real time PCR, and copy numbers were determined based on a standard curve that was produced using real time PCR on known quantities of purified RNA. Octylamine-modified beads bound and recovered approximately 300 to 500 more RNA transcripts than the non-modified beads, amounting to an approximate increase in RNA recovery of 10-15%.

TABLE 12
Recovery of Encapsulated DNA from beads modified using octylamine

| | | Amount | | Recovery - |
|---|---|---|---|---|
| | | recovered from | Amount recovered | modified beads |
| | | modified Sera- | from unmodified | relative to |
| | Max % COO⁻ | Mag ™ (avg # of | Sera-Mag (avg # | unmodified beads |
| Ligand | converted | copies) | of copies) | (%) |
| octylamine | 7 | 4068 | 3632 | 112 |
| octylamine | 33 | 6981 | 7030 | 99 |
| octylamine | 100 | 32 | 3992 | 1 |

(4) NH₂—C6-PO₃-TTTTTT-MGB: The ability of NH₂—C6-PO₃-TTTTTT-MGB-coupled carboxylated paramagnetic to purify HPIV transcript RNA also was determined using the methods described above. Aliquots of the eluted RNA were reverse transcribed and quantitated with real time PCR, and copy numbers were determined based on a standard curve that was produced using real time PCR on known quantities of purified RNA. NH₂—C6-PO₃TTTTTT-MGB-modified beads bound and recovered approximately 125 more RNA transcripts than the non-modified beads, amounting to an approximate increase in RNA recovery of 5%.

TABLE 13
Recovery of Encapsulated DNA from beads modified using NH₂—C6—PO₃TTTTTT-
MGB

| | | Amount | Amount | |
|---|---|---|---|---|
| | | recovered | recovered | |
| | | from | from | % recovery |
| | | modified | unmodified | from modified |
| | Max % | Sera-Mag ™ | Sera-Mag | beads relative |
| | COO⁻ | (avg # of | (avg # of | to unmodified |
| Ligand | converted | copies) | copies) | beads |
| NH₂(CH₂)₆—PO₃⁻-TTTTTT-PO₃⁻- | 20 | 2348 | 2286 | 103 |
| MGB | | | | |

iii. Recovery of Bacterial DNA

Beads were coupled to NH₂—C6-hexamine glycol-MGB as described in Example 1 and used to process bacterial as described in Example 5c.

TABLE 14
Recovery of Bacterial DNA from beads modified using NH₂—C6-
hexamine glycol-MGB

| | | Amount | Amount | |
|---|---|---|---|---|
| | | recovered | recovered | |
| | | from | from | % recovery |
| | | modified | unmodified | from modified |
| | Max % | Sera-Mag ™ | Sera-Mag | beads relative |
| | COO⁻ | (avg # of | (avg # of | to unmodified |
| Ligand | converted | copies) | copies) | beads |
| NH₂(CH₂)₆—PO₃⁻—(CH₂CH₂O)₆—PO₃⁻- | 5 | 12715 | 10626 | 120 |
| MGB | | | | |
| NH₂(CH₂)₆—PO₃⁻—(CH₂CH₂O)₆—PO₃⁻- | 5 | 12699 | 12554 | 101 |
| MGB | | | | |

iv. Recovery from Complex Samples

Beads were coupled to propylamine as described in Example 1 and used to isolate DNA from a complex sample as described in Example 5d. Propylamine-modified carboxylated paramagnetic beads displayed comparable purification characteristics compared to non-modified beads when DNA from a nasopharengeal swab collected in transport medium was isolated. Between 47 and 49% of the input RNA was recovered using modified beads, compared with 47 to 51% using non-modified beads.

TABLE 15
Recovery of 32P DNA spiked into nasopharengeal sample preparations

| | | | | | | % re- |
|---|---|---|---|---|---|---|
| | | Max | | | | covered |
| | | potential | | | | from |
| Spec- | | % | % | % | % | Sera- |
| imen | Ligand | converted | bound | eluted | recovery | Mag |
| NP-1 | Propylamine | 6.7 | 98 | 47 | 47 | 49 |
| NP-2 | Propylamine | 6.7 | 95 | 51 | 49 | 47 |
| NP-2 | Propylamine | 6.7 | 98 | 46 | 45 | 51 |

0.5 ml of transport medium from a negative nasopharyngeal specimen was pretreated with 2% lauroyl sarcosine and 15-30 u protease(Sigma-Aldrich, Mo.) for 10 min with shaking. The sample was then added to 1 ml lysis buffer containing either Sera-Mag or propylamine modified carboxylated paramagnetic beads and ³²P-DNA. The samples were processed as described above.

### EXAMPLE 7

### Comparative Evaluation of Efficiency and Recovery of Nucleic Acid Using Propylamine-Modified Carboxylated Paramagnetic Beads and Commercial Methods

The recovery of both RNA and DNA using propylamine-modified carboxylated paramagnetic beads was compared with the efficiency and recovery using one of three automated commercial sample preparation methods or a commercial manual spin column method. The four commercial systems used in these comparative studies included the MagNA Pure Compact System (Roche Applied Science), NucliSens® easyMAG® (bioMerieux), the BioRobot EZ1® System (Qiagen) and the QIAamp® MinElute® Virus Spin Columns (Qiagen).

Briefly, three nucleic acid templates were introduced into negative plasma and nasopharyngeal swab samples. The nucleic acid templates included Influenza A (FA) RNA transcript, Influenza A virus, and Legionella pneumophila genomic DNA. The nucleic acid was then extracted using the respective protocols, followed by reverse transcription. The samples were then amplified by PCR and the number of amplicons were detected and quantitated. The criteria for evaluation was the level of amplicons produced when the equivalent of 133-160 μl of sample containing low copy numbers was used in the amplification reactions.

1. Nucleic Acid Samples

Three nucleic acid templates were separately spiked plasma and nasopharyngeal swab samples; 1) transcripts from the matrix protein gene of Influenza A (FA); 2) Purified influenza A (H1N1) virus; and 3) purified Legionella pneumophila (LP) genomic DNA. The transcripts from the matrix protein gene of Influenza A (FA) were stored at −80° C. in aliquots of 1×10⁸ copies/μl prior to use. The purified influenza A (H1N1) virus (Advanced Biotechnologies Incorporated (ABI) was diluted in M4 media to 1×10⁸ viral particles/ml and aliquoted and stored at −80° C. prior to use. The purified genomic DNA was purchased from ATCC. It was then diluted to a final concentration of 2×10⁶ genomic copies/μl and aliquoted and frozen at −80° C. For each experiment a new aliquot of each sample was thawed and diluted.

Recovered plasma (1 unit) was purchased from the San Diego Blood Bank, and was aliquoted and frozen at −80° C. Nasopharyngeal swab specimens were tested in sub-pools to ensure that they were negative for the organisms being tested. The samples were then pooled and aliquoted and frozen in −80° C. A new aliquot was thawed and used for each experiment.

Each nucleic acid template was then introduced into each sample type to obtain 6 combination types:

1) Influenza A transcript in plasma

2) Influenza A transcript in nasopharyngeal swab sample

3) Influenza A virus in plasma

4) Influenza A virus spiked in nasopharyngeal swab sample

5) L. pneumophila genomic DNA in plasma

6) L. pneumophila genomic DNA in nasopharyngeal swab sample

For each combination type, 100, 1000 or 10,000 transcript copies, genomic copies or viral particles were introduced into the sample. Influenza A virus was introduced directly into the plasma or nasopharyngeal swab samples. Four hundred microliters of each sample containing the virus was then mixed with the appropriate lysis buffer for initiation of the purification process. To generate the Influenza A RNA transcript and L. pneumophila DNA samples, the plasma and nasopharyngeal swab samples were first mixed with the appropriate lysis buffer before the nucleic acid was introduced.

2. Purification

As noted above, 400 μl of each sample, in duplicate, was purified using each of the purification systems. The total number of runs performed for each sample for each method was as follows:

Propylamine-modified carboxylated beads: 5 runs

BioRobot EZ1® System: 21 runs

NucliSens® easyMAG®: 12 runs

MagNA Pure Compact System: 26 runs

QIAamp® MinElute® Virus Spin Columns: 10 runs

The purified nucleic acid was eluted in 50-60 μl of appropriate elution buffer. Thus, a seven to eight fold increase in concentration was obtained. A negative and a positive control were included in most runs. Negative samples were processed and amplified along with positive specimens to determine whether cross contamination occurred. Positive controls were prepared in runs that used purified RNA or DNA targets. The positive controls was established by adding 1000 or 10,000 copies per mL of target to an aliquot of a negative eluate and amplified.

For purification using the commercial systems, the purifications was performed according to manufacturer's instructions. Table 16 sets forth the instruments, automated protocols and kits that were used for each commercial system.

TABLE 16
Commercial purification systems

| Purification | | | Automated | |
|---|---|---|---|---|
| System | Company | Instrument | Protocol | Kit |
| BioRobot | Qiagen | EZ1 ® | Virus Purification | Virus mini kit |
| EZ1 ® System | | | Ver.2.0 | Ver.2.0 |
| NucliSens ® | Biomerieux | easyMAG ® | Generic 1.0.6 | NucliSENS |
| easyMAG ® | | | | easyMAG |
| | | | | reagents |
| MagNA Pure | Roche | MagNA | Total_NA_Plasma_100_400_V3_1 | MagNA Pure |
| Compact | | Pure | | Compact |
| System | | Compact | | Nucleic Acid |
| | | | | Isolation Kit I |
| QIAamp ® | Qiagen | Spin column | — | QIAamp |
| MinElute ® | | | | MinElute |
| Virus Spin | | | | Virus Spin Kit |
| Columns | | | | |

Purification using the propylamine-modified carboxylated beads was performed as described below.

i. Modification of Carboxylated Paramagnetic Beads Using Propylamine.

Propylamine-modified carboxylated beads were generated essentially as described in Example 1, with some modifications. Briefly, 50 mg carboxylated paramagnetic beads (24 μmol carboxylate) (Sera-Mag™ Microparticles, Seradyn, IN) were washed two times in 0.1 M MES buffer (2-(N-morpholino) ethanesulfonic acid coupling buffer-Research organics, OH), then resuspended in 0.5 M MES, 4 μmol propylamine or propylamine hydrochloride (hydrophobic ligand—Sigma-Aldrich Inc., MO) followed by the addition of 1.6 μmol coupling reagent, 1-ethyl-3-(3′-dimethylaminopropyl) carbodiimide (EDAC) (EMD Biosciences inc., CA). Water was added to achieve final concentration of 0.1 M MES and 4% w/v solution of beads. This coupling mixture was incubated at 65° C. for 3.5 hours with frequent mixing. The coupled beads were then washed 4 times; once in 0.1 M MES at 65° C., twice in water at 65° C. and once in water at room temperature. The coupled beads were then resuspended and stored in 10 mM Tris and 0.05% Proclin and 5% beads.

ii. Purification Using Propylamine-Modified Carboxylated Beads.

The nucleic acid samples were purified essentially as described in Example 5B, with some modifications. For purification of the L. pneumophila DNA and Influenza A RNA transcript, 400 μl of the negative nasopharangeal swab or plasma sample was added to 1 ml lysis buffer containing 2.5 M guanidinium thiocyanate, 50% isopropanol, 0.75% lauroyl sarcosine, 25 mM Tris, pH 7.4, and 1 μg/ml polyA RNA. This solution was the spiked with 5 μl of nucleic acid to reach the final concentration of 10,000 or 1000 or 100 genomic copies or transcript copies per ml. For purification of nucleic acid from Influenza A virus, the virus was added directly to 400 μl of the negative nasopharangeal swab or plasma sample and then mixed with 1 ml lysis buffer. To these DNA or RNA solutions, a 0.5 ml aliquot of M4 transport medium (Remel, Lenexa, Kans.) and 15 μl of a 5% w/v solution of propylamine-modified carboxylated beads was added. The mixture was vortexed gently for 4 minutes and the magnetic beads were collected at the side of the tube with a magnet. The lysis buffer was removed and the beads were washed twice with a wash buffer containing 1.67 M guanidinium isothiocyanate, 33% isopropanol, 0.5% lauroyl sarcosine and 16.7 mM Tris, pH 7.4. The beads were washed a second time with a wash buffer containing 70% ethanol, 10 mM KCl, 2 mM Tris pH 7.0 and 0.2 mM EDTA, pH 8.0. The second wash buffer was removed and the beads were air dried and then treated with 50 μl of an elution buffer containing 0.1×TE, pH 8.0 (1 mM Tris and 0.1 mM EDTA). The beads were heated to 65° C. and the nucleic acid eluted.

3. Amplification and Detection

Following elution, the eluate was subjected to a reverse transcription (RT) reaction in a 30 μl reaction. The 30 μl reaction contained 20 μl eluate, 1×PCR Buffer II (Applied Biosystems, Foster City, Calif. (ABI)), 4 mM dNTP (ABI,), 2.5 M random hexamers (ABI), 5 mM MgCl₂, 13 units RNase Inhibitor (ABI) and 75 units MMLV reverse transcriptase (ABI). The 20 μl eluate contained a maximum of 1600, 160 or 16 copies of nucleic acid template, based on a maximal purification efficiency using starting samples containing 10,000, 1,000 or 100 RNA copies or viral particles per ml, respectively. The RT reaction included a 5 minute step at 22° C., a 14 minute step at 42° C. and a 1 minute step at 95° C.

The entire RT reaction was used in a 50 μl multiplexed PCR amplification mix, which also included 0.25 M of each primer, X PCR buffer II, 3.5 mM MgCl₂, and 2.5 units Fast Start Taq DNA polymerase (Roche Applied Science, Indianapolis, Ind.). The amplification mix for the purified L. pneumophila genomic DNA was a 5 plex PCR mix that included primer sets for Legionella pneumophila (LP) (forward primer: SEQ ID NO:1; reverse primer: SEQ ID NO:2), Legionella micdadei (LM) (forward primer: SEQ ID NO:3; reverse primer: SEQ ID NO:4), Bordetella pertussis (BP) (forward primer: SEQ ID NO:5; reverse primer: SEQ ID NO:6), Chlamydophila pneumophila (Cpn) (forward primer: SEQ ID NO:7; reverse primer: SEQ ID NO:8) and MS2 bacteriophage (internal control) (forward primer: SEQ ID NO:9; reverse primer: SEQ ID NO:10). The amplification mix for the purified samples containing the Influenza A virus nucleic acid was an 8 plex PCR mix that included primer sets for (forward primer: SEQ ID NO:1; reverse primer: SEQ ID NO:2), Legionella micdadei (LM) (forward primer: SEQ ID NO:3; reverse primer: SEQ ID NO:4), Bordetella pertussis (BP) (forward primer: SEQ ID NO:5; reverse primer: SEQ ID NO:6), Chlamydophila pneumophila (Cpn) (forward primer: SEQ ID NO:7; reverse primer: SEQ ID NO:8), Influenza A virus (forward primer: SEQ ID NO:11; reverse primer: SEQ ID NO:12), Influenza B virus (forward primer: SEQ ID NO:13; reverse primer: SEQ ID NO:14), Respiratory Syncytial Virus A and B (RSV) (forward primer (A): SEQ ID NO:15; forward primer (B): SEQ ID NO:16; reverse primer (A and B): SEQ ID NO:17) and MS2 (forward primer: SEQ ID NO:9; reverse primer: SEQ ID NO:10). The amplifications were performed using the following protocol:

| | |
|---|---|
| 1 cycle: | 95° C. for 10 minutes |
| 2 cycles: | 95° C. for 1 minute |
| | 55° C. for 30 minutes |
| | 72° C. for 45 minutes |
| 38 cycles: | 5° C. for 15 minutes |
| | 60° C. for 15 minutes |
| | 72° C. for 30 minutes |
| 1 cycle: | 72° C. for 3 minutes |

The amplicons from each reaction were sized and quantitated by capillary electrophoresis using an automated LabChip® instrument (Model No. AMS 90 SE; Caliper Life Sciences). All protocols were performed using the manufacturer's instructions. The results from the different systems were then compared in a series of paired t-test analyses at 95% confidence levels. The number of data points in the two categories must be identical for paired t-tests. Thus, the average values listed in the tables below for this same instrument can vary depending on the number of data points that were considered.

4. Results

i. Purification of Influenza A Transcript from Plasma and Nasopharyngeal Samples

Tables 17 and 19 set forth the amount of amplicon (ng/μl) obtained following RT and PCR of the Influenza A transcript RNA purified from plasma and nasopharyngeal samples using each purification system. The table set forth exemplary purification runs for samples that originally contained 10⁴, 10³ and 10² transcript copies per mL. Tables 18 and 20 set forth the corresponding paired t-test analyses. The numbers in parenthesis are the mean values of all the data points obtained from 100 to 10,000 input copy levels. N/S indicates no significant difference was observed between the two groups.

TABLE 17
Recovery of Influenza A transcript from plasma samples

| | | | | | | | | | | | | QIAamp ® | |
|---|---|---|---|---|---|---|---|---|---|---|---|---|---|
| Copies | | | | | | | | | | | | MinElute ® | |
| per ml | Modified | | EZ1 ® | | NucliSens ® | | | | MagNA Pure | | | Virus Spin | |
| sample | beads | | System | | easyMAG ® | | | | Compact | | | Columns | |
| 10⁴ | 6.71 | 9.15 | 1.7 | 1.2 | 17.1 | 18.6 | 5.4 | 7.6 | 9.8 | 12 | 7.6 | 5.87 | 5.57 |
| 10⁴ | 7.34 | 9.87 | 1 | 4.5 | 13.7 | 18.8 | 6.3 | 5.8 | 11.2 | 14.4 | 5.2 | 5.05 | 9.73 |
| 10³ | 1.21 | 1.19 | 0 | 0.9 | 5.2 | 2.2 | 1.7 | 1.4 | 0 | 4.4 | 1.6 | 0.55 | 20.9 |
| 10³ | 2.74 | 2.95 | 0 | 0.3 | 3 | 1.8 | 1.9 | 1 | 0 | 4.7 | 2.5 | 1.79 | 0.97 |
| 10² | 0 | 0 | 0 | 0 | 1.1 | 0.32 | 0 | 0 | 0 | 0 | 0 | 0 | 0 |
| 10² | 0.44 | 0.31 | 0 | 0 | 0 | 0 | 0 | 0 | 0 | 0 | 0 | 0.24 | 0 |
| Run | 2 | 3 | 14 | 18 | 4 | 4 | 3 | 10 | 2 | 6 | 14 | 2 | 3 |
| No. | | | | | | | | | | | | | |

TABLE 18
Paired t-test at 95% confidence level

| | | | | QIAamp ® |
|---|---|---|---|---|
| | NucliSens ® | | | MinElute ® |
| | easyMAG ® | MagNA Pure | | Virus Spin |
| | (EM) | Compact | Modified beads | Columns |
| EZ1 ® System | EM (2.6) > | Compact (4.4) > | Modified beads | N/S |
| | EZ1 (0.8) | EZ1 (0.8) | (3.5) > EZ1 (0.8) | |
| NucliSens ® | — | N/S | N/S | N/S |
| easyMAG ® | | | | |
| MagNA Pure | — | — | N/S | N/S |
| Compact | | | | |
| Modified beads | — | — | — | Modified beads |
| | | | | (3.5) > Spin (2.7) |

TABLE 19
Recovery of Influenza A transcript from nasopharyngeal samples

| | | | | | | | | | | | QIAamp ® | |
|---|---|---|---|---|---|---|---|---|---|---|---|---|
| Copies | | | | | | | | | | | MinElute ® | |
| per ml | Modified | | EZ1 ® | | NucliSens ® | | | MagNA Pure | | | Virus Spin | |
| sample | beads | | System | | easyMAG ® | | | Compact | | | Columns | |
| 10⁴ | 1.8 | 3.1 | | | 6.1 | 3 | | 8 | 3 | 2.1 | 4.2 | 3.6 |
| 10⁴ | 2.2 | 1.9 | | 0 | 6.7 | 1.9 | 1.9 | 5.1 | 2.5 | 1.7 | 3.5 | 3.2 |
| 10⁴ | 2 | 2.2 | 1 | 0 | 7.1 | 4.5 | 3.5 | | 7.6 | 2.7 | 2.9 | 2.9 |
| 10³ | 0.4 | 0.3 | 0 | 0 | 0.8 | 0.9 | 0 | 0.7 | 0 | 0.6 | 1.9 | 0 |
| 10³ | 0.5 | 0.6 | 0 | | 0.7 | 0.7 | 0 | 2.5 | 0.7 | 0 | 2.7 | 0.4 |
| 10³ | 0.3 | 1.2 | | | 0.6 | 1.5 | 0 | | 0 | 0.7 | 3.1 | 0.3 |
| Run No. | 2 | 3 | 15 | 17 | 4 | 10 | 3 | 3 | 5 | 15 | 5 | 6 |

TABLE 20
Paired t-test at 95% confidence level

| | | | | QIAamp ® |
|---|---|---|---|---|
| | NucliSens ® | MagNA | | MinElute ® |
| | easyMAG ® | Pure | | Virus Spin |
| | (EM) | Compact | Modified beads | Columns |
| EZ1 ® | EM (1.7) > | N/S | Modified beads | Spin (2.3) > |
| System | EZ1 (0.17) | | (0.93) > | EZ1 (0.17) |
| | | | EZ1 (0.17) | |
| NucliSens ® | — | N/S | N/S | N/S |
| easyMAG ® | | | | |
| MagNA Pure | — | — | N/S | N/S |
| Compact | | | | |
| Modified | — | — | — | Spin (2.4) > |
| beads | | | | Modified beads |
| | | | | (1.4) |

ii. Purification of RNA from Influenza A Virus Particles from Plasma and Nasopharyngeal Samples

Tables 21 and 23 set forth the amount of amplicon (ng/μl) obtained following RT and PCR of the RNA from Influenza A viral particles that was purified from plasma and nasopharyngeal samples using each purification system. The tables set forth exemplary purification runs for samples that originally contained 10⁴, 10³ and 10² virus particles per mL. Tables 22 and 24 set forth the corresponding paired t-test analyses. The numbers in parenthesis are the mean values of all the data points obtained from 100 to 10,000 input copy levels. N/S means no significant difference was observed between the two groups.

Pooled data points from purification experiments using plasma and nasopharyngeal swab samples spiked with Influenza A virus also were analyzed using a paired t-test (Table 25).

TABLE 21
Recovery of RNA from Influenza A virus particles from plasma samples

| | | | | | | | QIAamp ® |
|---|---|---|---|---|---|---|---|
| Copies | Mod- | | | | | | MinElute ® |
| per ml | ified | EZ1 ® | | NucliSens ® | MagNA Pure | | Virus Spin |
| sample | beads | System | | easyMAG ® | Compact | | Columns |
| 10⁴ | 16.1 | 3.8 | 3.8 | 15 | 20.6 | 11.6 | 14.1 |
| 10³ | 8 | 0.83 | 0.83 | 5.8 | 10 | 6.3 | 6.4 |
| 10³ | 7.9 | 2.1 | | 6.9 | 11.6 | 4.8 | 2 |
| 10³ | 9.1 | 1.9 | | 6.5 | 12.9 | 5.6 | 6.2 |
| 10² | 1.3 | 0.15 | | 3 | 0 | 0.6 | 2 |
| 10² | 2.1 | 0 | | 2.1 | 2.35 | 1.1 | 2.4 |
| 10² | 4 | 0 | | 1.5 | 3.9 | 1.9 | 1.3 |
| Run | 1 | 20 | 21 | 10 | 12 | 13 | 4 |
| No. | | | | | | | |

TABLE 22
Paired t-test at 95% confidence level

| | | | | QIAamp ® |
|---|---|---|---|---|
| | NucliSens ® | | | MinElute ® |
| | easyMAG ® | MagNA Pure | | Virus Spin |
| | (EM) | Compact | Modified beads | Columns |
| EZ1 ® | EM (5.8) > | Compact | Modified beads | Spin (4.9) > |
| System | EZ1 (1.3) | (8.8) > | (6.9) > | EZ1 (1.3) |
| | | EZ1 (1.5) | EZ1 (1.3) | |
| NucliSens ® | — | N/S | N/S | N/S |
| easyMAG ® | | | | |
| MagNA Pure | — | — | N/S | N/S |
| Compact | | | | |
| Modified | — | — | — | N/S |
| beads | | | | |

TABLE 23
Recovery of RNA from Influenza A virus particles from
nasopharyngeal samples

| | | | | | | QIAamp ® |
|---|---|---|---|---|---|---|
| Copies | Mod- | | | | | MinElute ® |
| per ml | ified | EZ1 ® | | NucliSens ® | MagNA Pure | Virus Spin |
| sample | beads | System | | easyMAG ® | Compact | Columns |
| 10⁴ | 4.9 | 0 | 0 | 10.7 | 2.8 | 11.92 |
| 10³ | 1.2 | 0 | 0 | 2.1 | 0.82 | 3.95 |
| 10³ | 1.5 | 0.94 | | 3.5 | 0.33 | 3.27 |
| 10³ | 2.2 | 1.7 | | 5 | 1.8 | 3.62 |
| 10² | 0.63 | 0.6 | | 0 | 0 | 1.59 |
| 10² | 0.91 | 0 | | 0.73 | 0 | 0.97 |
| 10² | 0.73 | 2.22 | | 0.62 | 0 | 1.03 |
| Run No. | 1 | 19 | 21 | 7 | 9 | 1 |

TABLE 24
Paired t-test at 95% confidence level

| | | | | QIAamp ® |
|---|---|---|---|---|
| | NucliSens ® | MagNA | | MinElute ® |
| | easyMAG ® | Pure | | Virus Spin |
| | (EM) | Compact | Modified beads | Columns |
| EZ1 ® | N/S | N/S | N/S | N/S |
| System | | | | |
| NucliSens ® | — | N/S | N/S | N/S |
| easyMAG ® | | | | |
| MagNA Pure | — | — | Modified beads | Spin (3.8) > |
| Compact | | | (1.7) > | Compact (0.82) |
| | | | Compact (0.82) | |
| Modified | — | — | — | N/S |
| beads | | | | |

TABLE 25
Paired t-test at 95% confidence level (pooled Influenza virus
purification data)

| | | | | QIAamp ® |
|---|---|---|---|---|
| | NucliSens ® | | | MinElute ® |
| | easyMAG ® | MagNA Pure | | Virus Spin |
| | (EM) | Compact | Modified beads | Columns |
| EZ1 ® | EM (5.8) > | Compact | Modified beads | Spin (4.9) > |
| System | EZ1 (1.3) | (8.8) > | (6.9) > | EZ1 (1.3) |
| | | EZ1 (1.5) | EZ1 (1.3) | |
| NucliSens ® | — | N/S | N/S | N/S |
| easyMAG ® | | | | |
| MagNA Pure | — | — | N/S | N/S |
| Compact | | | | |
| Modified | — | — | — | N/S |
| beads | | | | |

iii. Purification of L. pneumophila Genomic DNA from Plasma and Nasopharyngeal Samples

Tables 26 and 28 set forth the amount of amplicon (ng/μl) obtained following RT and PCR of the L. pneumophila genomic DNA from that was purified from plasma and nasopharyngeal samples using each purification system. The tables set forth exemplary purification runs for samples that originally contained 10⁴, 10³ and 10² virus particles per mL. Tables 27 and 29 set forth the corresponding paired t-test analyses. The numbers in parenthesis are the mean values of all the data points obtained from 100 to 10,000 input copy levels. N/S means no significant difference was observed between the two groups.

TABLE 26
Recovery of L. pneumophila genomic DNA from plasma samples

| | | | | | | | | | MagNA | | QIAamp ® | |
|---|---|---|---|---|---|---|---|---|---|---|---|---|
| Copies | Mod- | | | | | | | | Pure | | MinElute ® | |
| per ml | ified | | EZ1 ® | | NucliSens ® | | | | Com- | | Virus Spin | |
| sample | beads | | System | | easyMAG ® | | | | pact | | Columns | |
| 10⁴ | 5.9 | 6.8 | | | 8.5 | 6.5 | 7.5 | 5.1 | 8.2 | 9.3 | 6.3 | 8.3 |
| 10⁴ | 6.3 | 5.9 | 8.1 | 9.5 | 7.5 | 6.6 | 7 | 7 | 9.1 | 8.3 | 6.4 | 8.6 |
| 10³ | 2.5 | 1.9 | 4.2 | 3.6 | 3.9 | 3.9 | 3.8 | 5.5 | 4.7 | 5.8 | 2.6 | 3.3 |
| 10³ | 1.9 | 2.5 | 3.7 | 3.7 | 4.2 | 3.8 | 3.1 | 4.5 | 4.7 | 5.4 | 2.8 | 2 |
| 10² | 0.5 | 0.4 | 1.4 | 1 | 1.4 | 1.5 | 1.2 | 0.7 | 1.1 | 0.9 | 0.2 | 0 |
| 10² | 0.2 | 0.2 | 0.9 | 0.55 | 0.5 | 0.8 | 0.4 | 0.6 | 0 | 0.5 | 0.8 | 0.7 |
| Run | 4 | 5 | 1 | 5 | 1 | 1 | 2 | 2 | 1 | 7 | 7 | 8 |
| No. | | | | | | | | | | | | |

TABLE 27
Paired t-test at 95% confidence level

| | | | | QIAamp ® |
|---|---|---|---|---|
| | NucliSens ® | | | MinElute ® |
| | easyMAG ® | MagNA Pure | | Virus Spin |
| | (EM) | Compact | Modified beads | Columns |
| EZ1 ® System | N/S | N/S | EZ1 (3.7) > | N/S |
| | | | Modified beads | |
| | | | (2.3) | |
| NucliSens ® | — | Compact (4.8) > | EM (4.1) > | N/S |
| easyMAG ® | | EM (4.1) | Modified beads | |
| | | | (2.9) | |
| MagNA Pure | — | — | Compact (4.8) > | Compact (4.8) > |
| Compact | | | Modified beads | Spin (3.5) |
| | | | (2.9) | |
| Modified beads | — | — | — | N/S |

TABLE 28
Recovery of L. pneumophila genomic DNA from nasopharyngeal
samples

| | | | | | | | | | | | | | QIAamp ® | |
|---|---|---|---|---|---|---|---|---|---|---|---|---|---|---|
| | | | | | | | | | | | | | MinElute ® | |
| Copies | | | | | | | | | | | | | Virus | |
| per ml | Modified | | EZ1 ® | | | NucliSens ® | | | | | | | Spin | |
| sample | beads | | System | | | easyMAG ® | | | MagNA Pure Compact | | | | Columns | |
| 10⁴ | 1.3 | 3.2 | | | | 6.3 | 6.3 | 6.6 | 7.3 | 9.4 | | 10.1 | 5.9 | 2.9 |
| 10⁴ | 1.5 | 3.7 | 3.7 | 5.7 | 4.8 | 5.8 | 3.9 | 6.8 | 8.3 | 6 | | 9.7 | 5.2 | 1.4 |
| 10³ | 1.3 | 3.3 | 4 | 5.7 | 4.8 | 5.3 | 3.8 | 6.3 | 8.1 | 6.8 | 9.4 | 9.9 | 5.3 | 2.2 |
| 10³ | 0.2 | 1 | 1.5 | 1.3 | 1.3 | 1.6 | 1.1 | 1.4 | 3.4 | 3.3 | 3.8 | 4.6 | 0.6 | 0.2 |
| 10² | 0.3 | 0.6 | 1.1 | 1.9 | 1.2 | 1.3 | 0.6 | 1.6 | 3.3 | 2 | | 2.9 | 0.8 | 0.5 |
| 10² | 0.4 | 1 | 1.9 | 2 | | 1.8 | 0.6 | 1.4 | 3.1 | 3.8 | | 3.5 | 1.8 | 0.3 |
| Run | 4 | 5 | 8 | 11 | 16 | 2 | 6 | 13 | 4 | 8 | 16 | 17 | 9 | 10 |
| No. | | | | | | | | | | | | | | |

TABLE 29
Paired t-test at 95% confidence level

| | | | | QIAamp ® |
|---|---|---|---|---|
| | NucliSens ® | | | MinElute ® |
| | easyMAG ® | MagNA Pure | | Virus Spin |
| | (EM) | Compact | Modified beads | Columns |
| EZ1 ® System | N/S | Compact (5.2) > | EZ1 (3.2) > | N/S |
| | | EZ1 (2.9) | Modified beads | |
| | | | (1.5) | |
| NucliSens ® | — | Compact (5.2) > | EM (3.4) > | EM (3.4) > |
| easyMAG ® | | EM (3.5) | Modified beads | Spin (2.3) |
| (EM) | | | (1.5) | |
| MagNA Pure | — | — | Compact (5.4) > | Compact (5.4) > |
| Compact | | | Modified beads | Spin (2.3) > |
| | | | (1.5) | |
| Modified beads | — | — | — | N/S |

5. Summary

Generally, purification of nucleic acid using propylamine-modified carboxylated beads resulted in similar recoveries of DNA and RNA to that observed when the nucleic acid was purified using the commercially available systems. Using propylamine-modified carboxylated bead for purification of RNA transcripts and RNA from virus was as efficient or better than observed when using the commercially available systems. In particular, recovery was greater when using the propylamine-modified carboxylated beads than when using the EZ1 system, which showed lower and inconsistent recovery of RNA targets. Thus, using propylamine-modified carboxylated beads results in equivalent nucleic acid recovery as is observed using more expensive systems that require instruments and/or more expensive reagents. In addition, the time taken to purify the nucleic acid is similar, while the maximum number of samples that can be purified per run in greater. Table 30 sets forth a comparison of some of the parameters of each purification system.

TABLE 30
Comparison of purification system parameters

| | | | | | QIAamp ® |
|---|---|---|---|---|---|
| | | | | MagNA | MinElute ® |
| | Modified | EZ1 ® | NucliSens ® | Pure | Virus Spin |
| | beads | System | easyMAG ® | Compact | Columns |
| Purification | 45 min | 43 min | 44-55 min | 30 min | 90 min |
| time | | | | | |
| Max # of | 48 | 6 | 24 | 8 | 20 |
| samples per | | | | | |
| run | | | | | |
| Protocol | Single | Contained | Integrated | Integrated | One kit |
| | protocol | on card to be | into the | into the | |
| | | inserted | instrument | instrument | |
| | | into | | | |
| | | instrument | | | |
| Eluate volume | multiple | single | multiple | single | multiple |
| within a run | | | | | |
| Reagent & | none | none | Bar code | Bar code | none |
| sample | | | | | |
| tracking | | | | | |
| Waste | Automatic, | Manual | Automatic at | Automatic, | Manual |
| disposal | At the time | | the time of | after | |
| | of | | extraction | extraction | |
| | extraction | | | | |

Since modifications will be apparent to those of skill in this art, it is intended that this invention be limited only by the scope of the appended claims.

What is claimed:

1. A method of isolating target nucleic acid molecules from a sample, comprising the steps of:
a) identifying a solid support;
b) adjusting the hydrophilicity of the solid support by adjusting the amount of hydrophilic ligand on the solid support;
c) adjusting the hydrophobicity of the solid support by adjusting the amount of hydrophobic ligand on the solid support; and
d) binding the target nucleic acid molecules from the sample onto the resulting solid support,
wherein the hydrophobicity of the resulting solid support is adjusted for binding the nucleic acid molecules onto the solid support and the hydrophilicity of the solid support is adjusted for maintaining colloidal stability of the solid support and for eluting the nucleic acid molecules off the solid support, whereby the amount of target nucleic acid(s) bound to the solid support and/or recovered after elution from the solid support is about 5% to about 500% greater than the amount of target nucleic acid(s) bound to the solid support and/or recovered from the solid support in the absence of either the at least one hydrophobic ligand or the at least one hydrophilic ligand.

2. The method of claim 1, wherein the hydrophilic and/or hydrophobic ligand(s) is/are a functional group of the material constituting the solid support.

3. The method of claim 1, wherein the hydrophilic and/or hydrophobic ligand(s) is/are operatively linked to the solid support.

4. The method of claim 3, wherein the hydrophobic ligand(s) is/are operatively linked to the hydrophilic ligand(s).

5. The method of claim 1, wherein a hydrophilic ligand is carboxylate.

6. The method of claim 1, wherein a hydrophobic ligand is an aliphatic amine.

7. The method of claim 3, wherein the hydrophilic ligand is a carboxylate, the hydrophobic ligand is an amine, the amine is operatively linked to the carboxylate and the operative linkage is via an amide bond.

8. The method of claim 4, wherein the percentage of hydrophilic ligand(s) that is/are operatively linked to the hydrophobic ligand(s) is from about 0.0001% to about 100% of total hydrophilic ligands.

9. The method of claim 6, wherein the aliphatic amine is selected from among propylamine, propylamine hydrochloride, octylamine, butoxypropylamine, butylamine, 2-(2-aminoethoxy)ethanol, NH₂(CH₂)ₖ—O-{(CH₂CH₂O)ₗ})ₘ-MGB, NH₂—(CH₂)₆—O-Tₙ-MGB, NH₂(CH₂)ₖ—O—(CH₂CH₂O)ₗ-Tₙ, NH₂(CH₂)₆—O—P(═O)(O⁻)—O—(CH₂CH₂O)₆—P(═O)(O⁻)—O-MGB and NH₂(CH₂)₆—O—P(═O)(O⁻)—O-TTTTTT-O—P(═O)(O⁻)—O-MGB, wherein:
k is an integer between 1 and 10;
l is an integer between 1 and 10;
m is an integer between 1 and 3;
n is an integer between 1 and 10;
T is thymidine; and
MGB is a DNA minor groove binder.

10. The method of claim 1, wherein the solid support is in the form of a bead.

11. The method of claim 10, wherein the bead is magnetic.

12. The method of claim 1, wherein the material comprising the solid support is selected from among agarose, cellulose, nitrocellulose, cellulose acetate, dextran, polysaccharides, glass, silica, gelatin, polyvinyl pyrrolidone, rayon, nylon, polyethylene, polypropylene, polybutylene, polycarbonate, polyesters, polyamides, vinyl polymers, polyvinyl alcohols, polystyrene, carboxylate-modified polystyrene, polystyrene cross-linked with divinylbenzene, acrylic resins, acrylates, acrylic acids, acrylamides, polyacrylamides, polyacrylamide blends, co-polymers of vinyl and acrylamide, methacrylates, methacrylate derivatives and co-polymers thereof.

13. The method of claim 12, wherein the material comprises carboxylate-modified polystyrene.

14. The method of claim 9, wherein the aliphatic amine is NH₂(CH₂)ₖ—O-{(CH₂CH₂O)ₗ}ₘ-MGB or NH₂(CH₂)₆—O-Tₙ-MGB, and the minor groove binder (MGB) is selected from among netropsin, distamycin, lexitropsin, mithramycin, chromomycin A3, olivomycin, anthramycin, sibiromycin, pentamidine, stilbamidine, berenil, CC-1065, Hoechst 33258, 4′-6-diamidino-2-phenylindole (DAPI),

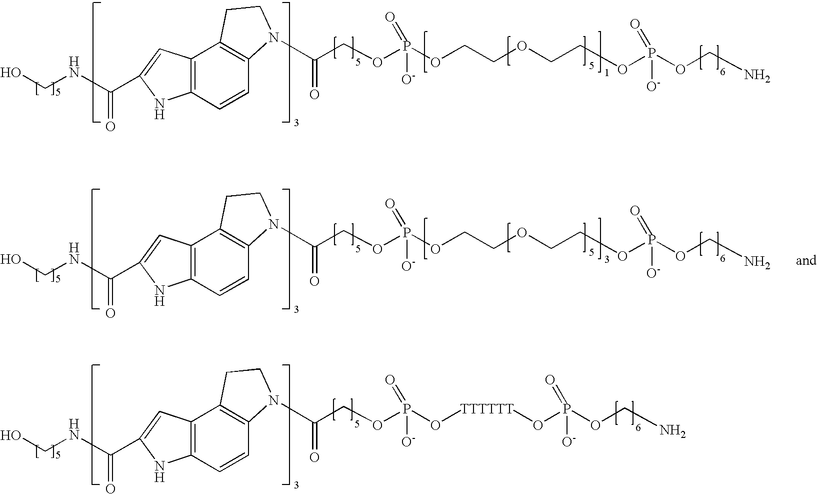

15. The method of claim 1, wherein the amount of hydrophobic ligand(s) on the solid support relative to the amount of hydrophilic ligands on the solid support is from or from about 0.0001% to or to about 100%, from or from about 0.003% to or to about 70%, from or from about 0.005% to or to about 65%, from or from about 0.01% to or to about 50%, from or from about 0.03% to or to about 40%, from or from about 0.03% to or to about 33%, from or from about 0.1% to or to about 20%, from or from about 0.5% to or to about 10%, from or from about 0.01% to or to about 5%, from or from about 0.001% to or to about 3%, from or from about 0.0001% to or to about 3%, or from or from about 0.005% to or to about 2%.

16. The method of claim 9, wherein the hydrophilic ligand is carboxylate and is operatively linked to the amine.

17. The method of claim 16, wherein the percentage of carboxylate residues operatively linked to the amine is from about or at 0.1% to about or at 100%.

18. The method of claim 17, wherein the amine is propylamine, propylamine hydrochloride, or octylamine.

19. The method of claim 18, wherein:
the amine is propylamine; and
the percentage of carboxylate residues operatively linked to propylamine is from about or at 0.1% to about or at 20%, from about or at 1% to about or at 10%, from about or at 1% to about or at 2%, from about or at 15% to about or at 20%, or from about or at 15% to about or at 17%.

20. The method of claim 18, wherein:
the amine is propylamine; and
the percentage of carboxylate residues operatively linked to propylamine is about or at 100%, about or at 16.7%, about or at 6.7% or about or at 1.7%.

21. The method of claim 18, wherein:
the amine is propylamine hydrochloride; and
the percentage of carboxylate residues operatively linked to propylamine hydrochloride is from about or at 1% to about or at 10%, or from about or at 1% to about or at 2%.

22. The method of claim 21, wherein the percentage of carboxylate residues operatively linked to propylamine hydrochloride is about or at 1.7%.

23. The method of claim 18, wherein:
the amine is octylamine; and
the percentage of carboxylate residues operatively linked to octylamine is from about or at 0.1% to about or at 20%, or from about or at 1% to about or at 10%.

24. The method of claim 23, wherein the percentage of carboxylate residues operatively linked to octylamine is about or is 6.7%.

25. The method of claim 17, wherein the amine is NH₂(CH₂)₆—O—P(═O)(O⁻)—O—(CH₂ CH₂O)₆—P(═O)(O⁻)—O-MGB, where MGB is:

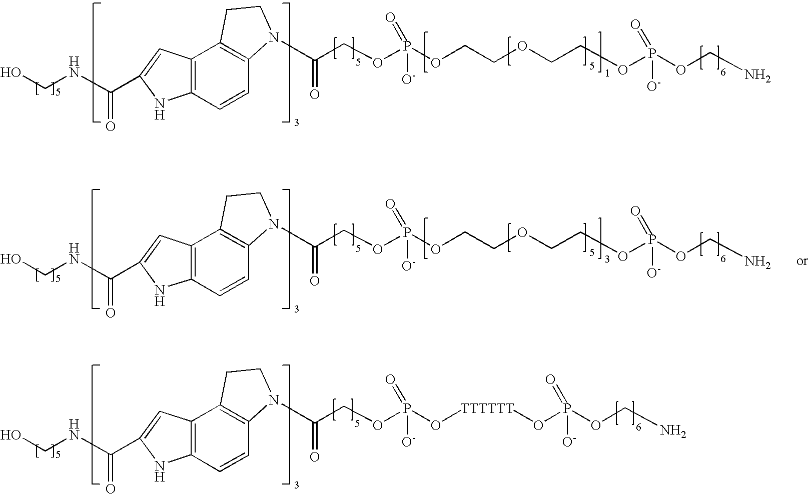

26. The method of claim 25, wherein the percentage of carboxylate residues operatively linked to the amine is from about or at 0.5% to about or at 5%.

27. The method of claim 26, wherein the percentage of carboxylate residues operatively linked to the amine is about or is 0.5%, is about or is 1%, or is about or is 2%.

28. The method of claim 16, wherein the operative linkage is a covalent linkage.

29. A method of preparing a solid support for isolating target nucleic acids from a sample, comprising:
identifying a solid support coated with a hydrophilic ligand; and
operatively linking a hydrophobic ligand to the identified solid support; wherein the hydrophobicity of the solid support is adjusted for binding the target nucleic acids from the sample onto the solid support, whereby the amount of target nucleic acid(s) bound to the solid support and/or recovered after elution from the solid support is about 5% to about 500% greater than the amount of target nucleic acid(s) bound to the solid support and/or recovered from the solid support in the absence of either the at least one hydrophobic ligand or the at least one hydrophilic ligand.

30. The method of claim 29, wherein the operative linkage is to the hydrophilic ligands on the solid support.

31. The method of claim 29, wherein the hydrophilic ligand is carboxylate.

32. The method of claim 29, wherein the operative linkage is achieved by forming an amide bond between the carboxylate group on the solid support and an amine group on the hydrophobic ligand.

33. A solid support comprising:
at least one hydrophilic ligand; and
at least one hydrophobic ligand,
wherein the amount of the one hydrophobic ligand on the solid support relative to the amount of the one hydrophilic ligand on the solid support is selected for binding target nucleic acid(s) from a sample onto the solid support and/or for eluting the bound target nucleic acid(s) from the solid support, whereby the amount of target nucleic acid(s) bound to the solid support and/or recovered after elution from the solid support is about 5% to about 500% greater than the amount of target nucleic acid(s) bound to the solid support and/or recovered from the solid support in the absence of either the one hydrophobic ligand or the one hydrophilic ligand; and
the amount of hydrophobic ligand(s) on the solid support relative to the amount of hydrophilic ligands on the solid support is from or from about 0.0001% to or to about 100%, from or from about 0.003% to or to about 70%, from or from about 0.005% to or to about 65%, from or from about 0.01% to or to about 50%, from or from about 0.03% to or to about 40%, from or from about 0.03% to or to about 33%, from or from about 0.1% to or to about 20%, from or from about 0.5% to or to about 10%, from or from about 0.01% to or to about 5%, from or from about 0.001% to or to about 3%, from or from about 0.0001% to or to about 3%, or from or from about 0.005% to or to about 2%.

34. The solid support of claim 33, wherein the ratio is from or from about 0.1% to or to about 20%.

35. The solid support of claim 33, comprising only one hydrophilic ligand and only one hydrophobic ligand.

36. The solid support of claim 33, wherein the hydrophobic ligand(s) is/are operatively linked to the hydrophilic ligand(s).

37. The solid support of claim 33, wherein a hydrophilic ligand is carboxylate.

38. The solid support of claim 33, wherein a hydrophobic ligand is an aliphatic amine.

39. The solid support of claim 35, wherein the hydrophilic ligand is a carboxylate, the hydrophobic ligand is an amine, the amine is operatively linked to the carboxylate and the operative linkage is via an amide bond.

40. The solid support of claim 36, wherein the percentage of hydrophilic ligand(s) that is/are operatively linked to the hydrophobic ligand(s) is from about 0.0001% to about 100%.

41. The solid support of claim 38, wherein the aliphatic amine is selected from among propylamine, propylamine hydrochloride, octylamine, butoxypropylamine, butylamine, 2-(2-aminoethoxy)ethanol, NH₂(CH₂)ₖ—O-{(CH₂ CH₂O)ₗ}ₘ-MGB, NH₂—(CH₂)₆—O-Tₙ-MGB, NH₂(CH₂)ₖ—O—(CH₂ CH₂O)ₗ-Tₙ, NH₂(CH₂)₆—O—P(═O)(O⁻)—O—(CH₂ CH₂O)₆—P(═O)(O⁻)—O-MGB and NH₂(CH₂)₆—O—P(═O)(O⁻)—O-TTTTTT-O—P(═O)(O⁻)—O-MGB, wherein:
k is an integer between 1 and 10;
l is an integer between 1 and 10;
m is an integer between 1 and 3;
n is an integer between 1 and 10;
T is thymidine; and
MGB is a DNA minor groove binder.

42. The solid support of claim 33, that is in the form of a bead.

43. The solid support of claim 42, wherein the bead is magnetic.

44. The solid support of claim 33, wherein the material comprising the solid support is selected from among agarose, cellulose, nitrocellulose, cellulose acetate, dextran, polysaccharides, glass, silica, gelatin, polyvinyl pyrrolidone, rayon, nylon, polyethylene, polypropylene, polybutylene, polycarbonate, polyesters, polyamides, vinyl polymers, polyvinyl alcohols, polystyrene, carboxylate-modified polystyrene, polystyrene cross-linked with divinylbenzene, acrylic resins, acrylates, acrylic acids, acrylamides, polyacrylamides, polyacrylamide blends, co-polymers of vinyl and acrylamide, methacrylates, methacrylate derivatives and co-polymers thereof.

45. The solid support of claim 44, wherein the material comprises carboxylate-modified polystyrene.

46. The solid support of claim 41, wherein the aliphatic amine is NH₂(CH₂)ₖ—O-{(CH₂CH₂O)ₗ}ₘ-MGB or NH₂(CH₂)₆—O-Tₙ-MGB, and the minor groove binder (MGB) is selected from among netropsin, distamycin, lexitropsin, mithramycin, chromomycin A3, olivomycin, anthramycin, sibiromycin, pentamidine, stilbamidine, berenil, CC-1065, Hoechst 33258, 4′-6-diamidino-2-phenylindole (DAPI),

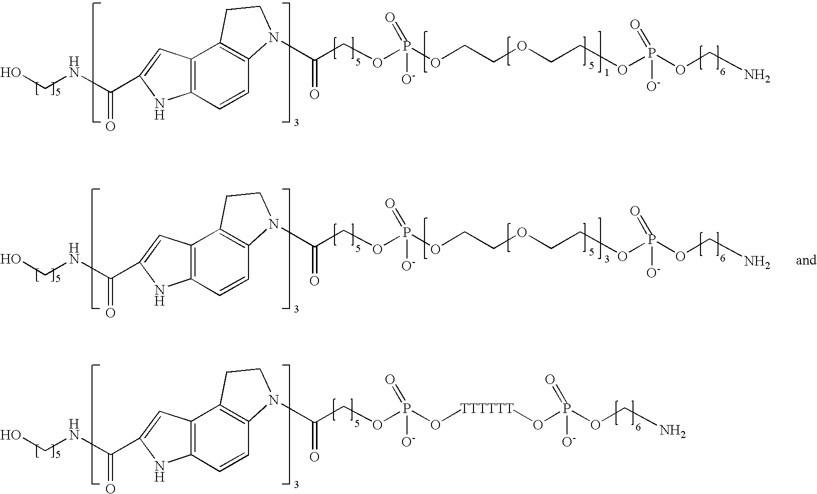

47. The solid support of claim 40, wherein the hydrophilic ligand is carboxylate and is operatively linked to the amine.

48. The solid support of claim 47, wherein the percentage of carboxylate residues operatively linked to the amine is from about or at 0.1% to about or at 100%.

49. The solid support of claim 48, wherein the amine is propylamine, propylamine hydrochloride or octylamine.

50. The solid support of claim 49, wherein:
the amine is propylamine; and
the percentage of carboxylate residues operatively linked to propylamine is from about or at 0.1% to about or at 20%, from about or at 15% to about or at 20%, from about or at 15% to about or at 17%, from about or at 1% to about or at 10%, or from about or at 1% to about or at 2%.

51. The solid support of claim 49, wherein:
the amine is propylamine; and
the percentage of carboxylate residues operatively linked to propylamine is at or about 100%, at or about 16.7%, at or about 6.7%, or at or about 1.7%.

52. The solid support of claim 49, wherein:
the amine is propylamine hydrochloride; and
the percentage of carboxylate residues operatively linked to propylamine hydrochloride is from about or at 1% to about or at 10% or from about or at 1% to about or at 2%.

53. The solid support of claim 52, wherein the percentage of carboxylate residues operatively linked to propylamine hydrochloride is 1.7%.

54. The solid support of claim 49, wherein:
the amine is octylamine; and
the percentage of carboxylate residues operatively linked to octylamine is from about or at 0.1% to about or at 20% or from about or at 1% to about or at 10%.

55. The solid support of claim 54, wherein the percentage of carboxylate residues operatively linked to octylamine is 6.7%.

56. The solid support of claim 48, wherein the amine is NH₂(CH₂)₆—O—P(═O)(O⁻)—O—(CH₂ CH₂O)₆—P(═O)(O⁻)—O-MGB, where MGB is:

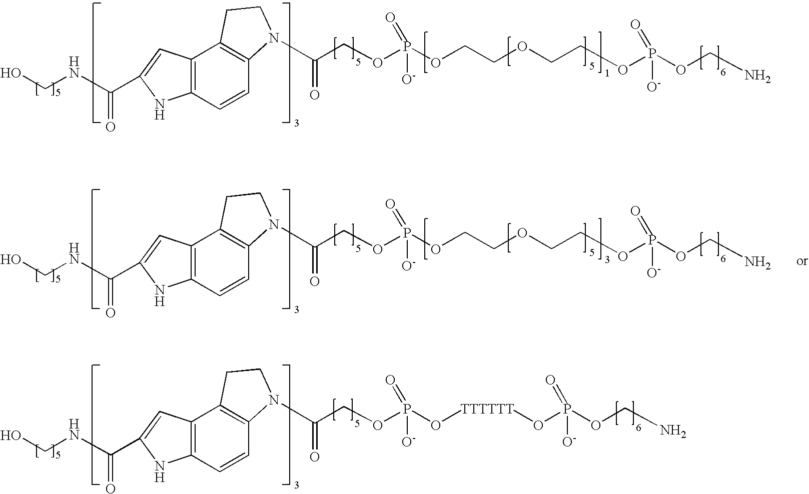

57. The solid support of claim 56, wherein the percentage of carboxylate residues operatively linked to the amine is from about or at 0.5% to about or at 5%.

58. The solid support of claim 57, wherein the percentage of carboxylate residues operatively linked to the amine is or is about 0.5%, is or is about 2%, or is or is about 1%.

59. The solid support of claim 48, wherein the amine NH₂(CH₂)₆—O—P(═O)(O⁻)—O-TTTTTT-O—P(═O)(O⁻)—O-MGB, where MGB is:

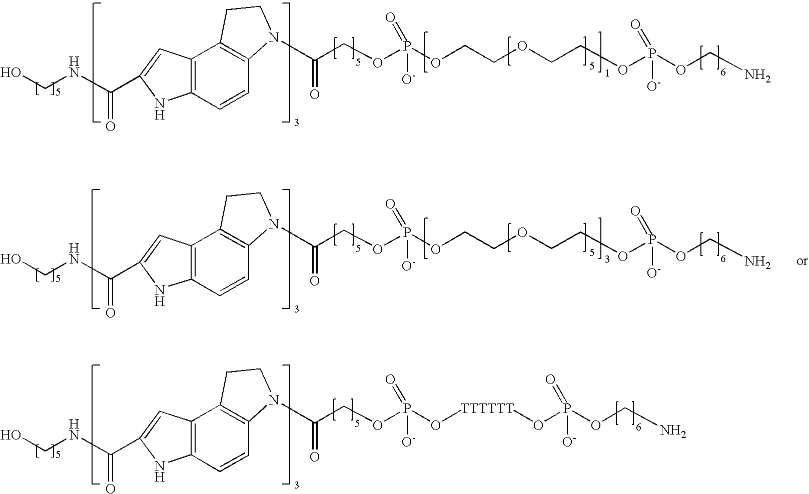

60. The solid support of claim 59, wherein the percentage of carboxylate residues operatively linked to the amine is from about or at 5% to about or at 20%.

61. The solid support of claim 47, wherein the operative linkage is a covalent linkage.

62. A method of isolating target nucleic acid molecules from a sample, comprising:
a) contacting a solid support of claim 33 with a sample comprising the target nucleic acid molecules; and
b) eluting the nucleic acid molecules.

63. The method of claim 62, further comprising after step a) and before step b);
i) mixing the components of a) in a solution containing a chaotropic buffer and alcohol, wherein the amounts of the chaotropic substance and alcohol are adjusted for binding the target nucleic acid molecules onto the solid support;
ii) separating the solid support containing the bound target nucleic acid molecules from the solution in i); and
iii) washing the solid support containing bound target nucleic acid molecules without eluting the target nucleic acid molecules.

64. The method of claim 62 that is a method of purifying target nucleic acid molecules from a sample.

65. The method of claim 62 that is a method of separating target nucleic acid molecules from each other according to type, length or sequence, wherein the amount or concentration of the chaotropic substance and/or the alcohol and/or the elution buffer is adjusted so that the target nucleic acid molecules are eluted sequentially according to type, length or sequence.

66. The method of claim 63, wherein the alcohol is polyethylene glycol.


SEQUENCE LISTING (17 sequences)

SEQ ID NO: 1 - DNA, 19 bp, Artificial Sequence
  Legionella pneumophila forward primer
tgcaatgtcaacagcaatg

SEQ ID NO: 2 - DNA, 27 bp, Artificial Sequence
  Legionella pneumophila reverse primer
ctcatagcgtcttgcatgcctttagcc

SEQ ID NO: 3 - DNA, 34 bp, Artificial Sequence
  Legionella micdadei forward primer
gttgctgaccgacgaccagatgaaggatgtgcta

SEQ ID NO: 4 - DNA, 24 bp, Artificial Sequence
  Legionella micdadei reverse  primer
tattccacagtaacgacgtcatcc

SEQ ID NO: 5 - DNA, 24 bp, Artificial Sequence
  Bordetella pertussis forward primer
atcgggcatgcttatgggtgttca

SEQ ID NO: 6 - DNA, 26 bp, Artificial Sequence
  Bordetella pertussis reverse  primer
gcgggctaactgtgaagattcaatag

SEQ ID NO: 7 - DNA, 20 bp, Artificial Sequence
  Chlamydia pneumoniae forward primer
agggctataaaggcgttgct

SEQ ID NO: 8 - DNA, 27 bp, Artificial Sequence
  Chlamydia pneumoniae reverse  primer
catgcagttaagtttaaaacagctgta

SEQ ID NO: 9 - DNA, 24 bp, Artificial Sequence
  MS2 forward primer
tgacttacatcgaagtgccgcaga

SEQ ID NO: 10 - DNA, 24 bp, Artificial Sequence
  MS2 reverse primer
aaccaaccgaactgcaactccaac

SEQ ID NO: 11 - DNA, 23 bp, Artificial Sequence
  Influenza A forward primer
cttctaaccgaggtcgaaacgta

SEQ ID NO: 12 - DNA, 23 bp, Artificial Sequence
  Influenza A reverse primer
acaaagcgtctacgctgcagtcc

SEQ ID NO: 13 - DNA, 26 bp, Artificial Sequence
  Influenza B forward primer
atggccatcggatcctcaactcactc

SEQ ID NO: 14 - DNA, 25 bp, Artificial Sequence
  Influenza B reverse primer
tcatgtcagctattatggagctgtt

SEQ ID NO: 15 - DNA, 19 bp, Artificial Sequence
  RSVA forward primer
agtccaaatggagcctgaa

SEQ ID NO: 16 - DNA, 32 bp, Artificial Sequence
  RSVB forward primer
aactaacccatccaaactaagctattcctcaa

SEQ ID NO: 17 - DNA, 20 bp, Artificial Sequence
  RSV reverse primer
gtgtatttgctggatgacag